(19) United States
(12) Patent Application Publication
(10) Pub. No.: US 2012/0129835 A1
(43) Pub. Date: May 24, 2012

(54) SCHIZOPHRENIA METHODS AND COMPOSITIONS

(75) Inventors: Kristen Brennand, Del Mar, CA (US); Fred H. Gage, La Jolla, CA (US)

(73) Assignee: SALK INSTITUTE FOR BIOLOGICAL STUDIES, La Jolla, CA (US)

(21) Appl. No.: 13/298,246

(22) Filed: Nov. 16, 2011

Related U.S. Application Data
(60) Provisional application No. 61/414,380, filed on Nov. 16, 2010.

(51) Int. Cl. A61K 31/553; C12Q 1/06; C40B 30/04; C12Q 1/68; A61K 31/5415; C12Q 1/70; A61P 25/18; A61K 31/5513; A61K 31/519; G01N 33/566; C12Q 1/02

(52) U.S. Cl. 514/211.13; 435/7.1; 435/39; 506/9; 435/6.12; 435/29; 435/5; 514/220; 514/259.41; 514/225.5

(57) ABSTRACT

Methods of preparing and using neural cells derived from human induced pluripotent stem cell (hiPSCs), particularly hiPSCs derived from subjects with schizophrenia are provided. The hiPSC-derived neural cells can be used to screen test compounds and to identify schizophrenia marker functions. The hiPSC-derived neural cells can be used to diagnose and/or assess the severity of schizophrenia in a subject. Further, may the hiPSC-derived neural cells from a subject be used as an in vitro system to identify the most effective candidate among existing drugs for that specific subject (i.e. personalized medicine).

### CROSS-REFERENCES TO RELATED APPLICATIONS

This application claims the benefit of U.S. Provisional Application No. 61/414,380 filed Nov. 16, 2010, which is hereby incorporated in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 92150-824615_ST25.TXT, created on Nov. 8, 2011, 17,990 bytes, machine format IBM-PC, MS-Windows operating system, is hereby incorporated by reference in its entirety and for all purposes.

### BACKGROUND OF THE INVENTION

Schizophrenia is now believed to be a developmental disorder with late manifestation of its characteristic symptoms. Onset is typically in adolescence or early adulthood, occasionally in childhood. 1.1% of the population over the age of 18 suffers from schizophrenia. See Association, A. P. Diagnostic and statistical manual of mental disorders: DSM-IV. 3rd ed., Rev. Edn., Vol. 4th ed., American Psychiatric Press, 1994. Schizophrenia often results in premature death from poverty, homelessness, substance abuse and poor health maintenance (Brown, S., Inskip, H. & Barraclough, B., 2000, Br J Psychiatry 177:212-217); the life expectancy of schizophrenic patients is up to ten years less than the general population (Hannerz, H., Borga, P. & Borritz, M., 2001, Public Health 115:328-337).

There is a strong genetic component to schizophrenia, with an estimated heritability of 80-85% (Cardno, A. G. & Gottesman, I I., 2000, Am J Med. Genet. 97:12-17; Sullivan, P. F., Kendler, K. S. & Neale, M. C., 2003, Arch Gen Psychiatry 60:1187-1192). Single nucleotide polymorphisms (SNPs), common polygenic variation involving thousands of alleles of very small effect, account for nearly 30% of the genetic variance of schizophrenia (Shi, J. et al., 2009, Nature 460:753-757; Stefansson, H. et al., 2009, Nature 460:744-747; Purcell, S. M. et al., 2009, Nature 460:748-752). Copy number variants (CNVs), rare structural disruptions of genes, such as ERBB4 (Walsh, T. et al., 2008, Science 320:539-543) or NRXN1 (Kirov, G. et al., 2008, Human Molecular Genetics 17:458-465), or regions, including 1q21.1, 15q11.2, 15q13.3, 16p11.2, 22q11.2 (Walsh, T. et al., 2008, Science 320:539-543; Mefford, H. C. et al., 2008, The New England Journal of Medicine 359:1685-1699; Stefansson, H. et al., 2008, Nature 455:232-236; Nature 455:237-241 (2008)), are highly penetrant but account for only 20% of cases. Numerous disruptions in any number of key neurodevelopmental pathways may be sufficient to produce a diseased state that could ultimately manifest as schizophrenia.

Postmortem studies of schizophrenic brain tissue have observed reduced volume (Bogerts, B. et al., 1990, Schizophr Res. 3:295-301), cell size (Zaidel, D. W., Esiri, M. M. & Harrison, 1997, P. J., Am J Psychiatry 154:812-818 (1997), spine density (Glantz, L. A. & Lewis, D. A., 2000, Arch Gen Psychiatry 57:65-73; Hill, J. J., Hashimoto, T. & Lewis, D. A., 2006, Molecular psychiatry 11:557-566 (2006)) and pyramidal cell disarray (Jonsson, S. A et al., 1997, Eur Arch Psychiatry Clin Neurosci. 247:120-127 (1997); Conrad, A. J. et al., 1991, Arch Gen Psychiatry 48:413-417) in the hippocampus and reduced cortical thickness (Selemon, L. D., Rajkowska, G. & Goldman-Rakic, P., 1998, J Comp Neurol. 392:402-412), cell size (Pierri, J. N. et al., 2003, Biol Psychiatry 54:111-120) and abnormal neural distribution (Vogeley, K. et al., 2000, Am J Psychiatry 157:34-39) in the prefrontal cortex. Neuropharmacological studies have implicated dopaminergic, glutamatergic and GABAergic activity in schizophrenia (Javitt, D. C. et al., Nat Rev Drug Discov. 7:68-83). The cell type affected in schizophrenia and the molecular mechanisms underlying the disease state remains unclear.

There is a need in the art for methods of directly reprogramming fibroblasts from schizophrenic patients into hiPSCs and subsequently differentiating these disorder-specific hiPSCs into neurons as well as cell-based models permitting the characterization of complex genetic psychiatric diseases using hiPSCs. Provided herein are solutions to these and other needs in the art, by, inter alia, identifying neural phenotypes and gene expression changes associated with schizophrenic neurons in vitro, advancing the field of hiPSC-based disease modeling and developing a transformative new tool with which to study schizophrenia.

### BRIEF SUMMARY OF THE INVENTION

Provided herein are, inter alia, methods of preparing and using neural cells derived from human induced pluripotent stem cell (hiPSCs), particularly hiPSCs derived from subjects with schizophrenia. The hiPSC-derived neural cells can be used to screen test compounds and to identify schizophrenia marker functions. By creating hiPSC-derived neural cells from a subject, one can use the hiPSC-derived neural cells to diagnose and/or assess the severity of schizophrenia in that subject. Further, may the hiPSC-derived neural cells from a subject be used as an in vitro system to identify the most effective candidate among existing drugs for that specific subject (i.e. personalized medicine).

In one aspect, a method of determining whether a test compound is capable of improving a schizophrenia marker function in a hiPSC-derived neural cell is provided. The method includes contacting a test compound with a hiPSC-derived neural cell derived from a schizophrenic subject. The hiPSC-derived neural cell exhibits a schizophrenia marker function at a first level in the absence of the test compound. Then, a second level of the schizophrenia marker function is determined in the presence of the test compound. The second level is compared to a control level. A smaller difference between the second level and the control level than between the first level and the control level indicates that the test compound is capable of improving the schizophrenia marker function.

In another aspect, a method of determining whether a subject is schizophrenic is provided. The method includes determining a level of a schizophrenia marker function in a hiPSC-derived neural cell derived from a subject and comparing the level to a control level. A difference between the determined level and the control level indicates that the subject is schizophrenic.

In another aspect, a method of identifying a schizophrenia marker function is provided. The method includes obtaining a cell from a schizophrenic subject and reprogramming the cell thereby forming a hiPSC. The hiPSC is allowed to differentiate thereby forming a hiPSC-derived neural cell derived from the schizophrenic subject. A level of a function of the hiPSC-derived neural cell is determined and the level is compared to a control level. A difference between the level and the control level indicates the function is a schizophrenia marker function.

In one aspect, a method of determining whether a schizophrenic subject is responsive to treatment with a loxapine compound is provided. The method includes contacting a loxapine compound with a hiPSC-derived neural cell. The hiPSC-derived neural cell is derived from the schizophrenic subject, and the hiPSC-derived neural cell exhibits a loxapine marker function at a first level in the absence of a loxapine compound. Then a second level of the loxapine marker function is determined and the second level is compared to a control level. A smaller difference between the second level and the control level than between the first level and the control level indicates the schizophrenic subject is responsive to treatment with a loxapine compound.

In another aspect, a method of determining whether a test compound is capable of improving a loxapine marker function is provided. The method includes contacting a test compound with a hiPSC-derived neural cell. The hiPSC-derived neural cell is derived from a schizophrenic subject, and the hiPSC-derived neural cell exhibits a loxapine marker function at a first level in the absence of the test compound. Then a second level of the loxapine marker function determined and the second level is compared to a control level. A smaller difference between the second level and the control level than between the first level and the control level indicates the test compound is capable of improving the loxapine marker function.

### BRIEF DESCRIPTION OF THE DRAWINGS

FIG. 1. Patient-specific hiPSCs, NPCs and neurons. Left panel FIG. 1: hiPSCs express NANOG and TRA-1-60. DAPI. ×100, scale bar 100 μm. Centre panel FIG. 1: hiPSC neural progenitor cells (NPCs) express NESTIN and SOX2 and DAPI. ×600, scale bar 100 μm. Right panel FIG. 1: hiPSC neurons express βIII-tubulin and the dendritic marker MAP2AB and DAPI. ×200, scale bar 100 μm.

FIG. 2. Decreased neural connectivity in schizophrenic hiPSC-derived neurons. FIG. 2A. Representative images of control and SCZD hiPSC neurons cotransduced with LV-SYNP-HTG and Rabies-ENVAΔG-RFP, 10 days post rabies transduction. All images were captured using identical laser power and gain settings. βIII-tubulin staining of the field is shown below each panel. ×400, scale bar 80 μm. FIG. 2B. Histogram showing treatment of SCZD hiPSC neurons with Loxapine resulted in a statistically significant improvement in neuronal connectivity. Error bars are s.e. (standard error), *P<0.05

FIG. 3. Decreased neurites and synaptic density but normal calcium transient activity in schizophrenic hiPSC-derived neurons. FIG. 3A: Histogram showing decreased neurites in SCZD hiPSC neurons. FIG. 3B. Histogram showing decreased PSD95 protein relative to MAP2AB for SCZD hiPSC neurons. FIG. 3C. Histogram showing a trend of decreased PSD95 synaptic density in SCZD hiPSC neurons. FIG. 3D-G. Electrophysiological characterization. hiPSC neurons cultured on astrocytes show normal sodium and potassium currents when voltage-clamped (FIG. 3D), normal induced action potentials when current-clamped (FIG. 3E), and spontaneous excitatory (FIG. 3F) and inhibitory (FIG. 3G) synaptic activity. FIG. 3H-K. Spontaneous calcium transient imaging. Representative spontaneous Fluo-4AM calcium traces of fluorescent intensity versus time generated from three-month-old hiPSC neurons (FIG. 3H). Histogram showing no difference between the spike amplitude of spontaneous calcium transients of control and SCZD hiPSC neurons (FIG. 3I). Histogram showing no difference between the total numbers of spontaneous calcium transients per total number of ROIs in cultures of control and SCZD hiPSC neurons (FIG. 3J). Histogram showing no change in percentage synchronicity per calcium transient in control and SCZD hiPSC neurons (FIG. 3K). Error bars are SE. Asterisks used as follows: *** p<0.001.

FIG. 4. RNA expression analysis of control and schizophrenic hiPS-derived neurons. Heat maps showing microarray expression profiles of altered expression of glutamate receptors (FIG. 4A), cAMP signaling (FIG. 4B), and WNT signaling (FIG. 4C) genes in SCZD hiPSC neurons. Fold-change and p-values (diagnosis) provided to the right of each heat map. FIG. 4D. Heat maps showing perturbed expression of NRG1 and ANK3 in all four SCZD patients, as well as altered expression of ZNF804A, GABRB1, ERBB4, DISC1 and PDE4B in some but not all patients. Fold-change and p-values (diagnosis) provided to the right of each heat map. FIG. 4E. Altered expression of NRG1 is detected in SCZD hiPSC neurons but not in patient fibroblasts, hiPSCs or hiPSC NPCs. FIG. 4F. qPCR validation of altered expression of NRG1, GRIK1, ADCY8, PRKCA, WNT7A, TCF4 and DISC1, as well as response to three weeks of treatment with Loxapine (striped bars) in six-week-old hiPSC neurons. Asterisks used as follows: * p<0.05, ** p<0.01, *** p<0.001.

FIG. 5. Reprogramming of patient fibroblasts to hiPSCs. FIG. 5A. Experimental schematic for generation of hiPSCs using doxycycline-inducible lentiviral reprogramming vectors. A constitutive CAGGs-rtTA lentivirus drives doxycycline-inducible expression of OCT4, SOX2, KLF4, cMYC, LIN28 and GFP. FIG. 5B, top panel. hiPSCs express NANOG and TRA-1-60. 400×, scale bar 80 μm. FIG. 5B, bottom panel. hiPSCs co-express the transcription factors SOX2 and OCT4. 400×, scale bar 80 μm. FIG. 5C. Representative teratoma assay for pluripotency. Teratomas containing all three germ layers were generated from every hiPSC-line utilized. 200×, scale bar 80 μm. FIG. 5D. Karyotyping of hiPSCs from SCZD and control patients revealed that all patients had normal karyotypes except for patient 2, who had an inversion of chromosome 1 between 1p13.3 and 1q13 that was also present in the HF. FIG. 5E. qPCR analysis of endogenous and lentiviral (LV) pluripotency gene expression in every control and SCZD HF, hiPSC and hiPSC NPC line was performed. Transcripts specific to lentiviral OCT4, SOX2, KLF4 and cMYC were not detected in hiPSC or NPC cell lines.

FIG. 6. Patient-specific hiPSCs, NPCs and neurons. FIG. 6A. Family pedigrees of patients. FIG. 6B. FACS analysis shows ˜80% of hiPSC NPC lines differentiate to MIL tubulin+ cells. Error bars are SE. FIG. 6C. Brightfield images of hiPSC neural differentiation. 100×, scale bar 100 μm. FIG. 6D, top panel. hiPSCs express NANOG and TRA-1-60. DAPI. 100×, scale bar 100 μm. FIG. 6D, middle panel. hiPSC neural progenitor cells (NPCs) express NESTIN and SOX2. 600×, scale bar 100 μm. FIG. 6D, bottom panel. hiPSC neurons express βIII-tubulin and the dendritic marker MAP2AB. 200×, scale bar 100 μm.

FIG. 7. Controls validating observations of decreased neuronal connectivity in SCZD hiPSC neurons. FIG. 7A. Schematic of rabies trans-neuronal tracing. Feature number legend: 301=hiPSC neurons cannot be transduced by ENVA serotyped rabies virus; 302=primary transduction with LV-SYNP-HTG causes expression of TVA receptor; 303=rabies-EnvAΔG-RFP transduces only the cells expressing LV-SYNP-HTG; 304=rabies ENVAΔG-RFP expression in LV-SYNP-HTG labeled hiPSC neuron; 305=monosynaptic transmission of rabies-ENVAΔG-RFP in retrograde direction only; 306=trans-neuronal labeling is measured as the ration of red:green hiPSC neurons. FIG. 7B. Representative images of control and SCZD hiPSC neurons cotransduced with LV-SYNP-HTG and Rabies-ENVAΔG-RFP, 10 days post rabies transduction. All images were captured using identical laser power and gain settings. PHI-tubulin staining of the field is shown below each panel. 400×, scale bar 80 μm. FIG. 7C. Histogram showing relative pixels of control and relative pixels of SCZD hiPSC neurons. hiPSC neurons were transduced with Rabies-ENVAΔG-RFP and either LV-SYNP-HTG or LV-SYNP-HT and assayed either 5, 7 or 10 days post Rabies-ENVAΔG-RFP transduction. Defects in SCZD hiPSC neuronal connectivity are more apparent with time post-rabies transduction, likely reflecting the signal amplification that occurs across the neuronal network when Rabies-ENVAΔG-RFP travels from one SYNP-HTG neuron to a second SYNP-HTG neuron. Error bars are SE. Asterisks used as follows: * p<0.05, *** p<0.001. FIG. 7D. Representative images of control and SCZD hiPSC neurons sequentially transduced with LV-SYNP-HTG and Rabies-ENVAΔG-RFP, or LV-SYNP-HT and Rabies-ENVAΔG-RFP, or Rabies-ENVAΔG-RFP alone. Images taken 10 days post rabies transduction. 400×, scale bar 80 μm. FIG. 7E. Functionally immature one-month-old hiPSC neurons are capable of trans-neuronal tracing. 400×, scale bar 80 μm. FIG. 7F. Representative images demonstrating that trans-neuronal tracing occurs even in the presence of three drugs known to affect synaptic transmission: tetradotoxin, KCl and ryanodine. 400×, scale bar 80 μm.

FIG. 8. Ability of antipsychotic medications to ameliorate decreased neuronal connectivity in SCZD hiPSC neurons. FIG. 8A. Representative images showing improved neuronal connectivity in SCZD three-month-old hiPSC neurons following three-week culture with loxapine. Images taken 10 days post rabies transduction. 200×, scale bar 200 μm. FIG. 8B and FIG. 8C. Histograms showing FACS analysis of control and SCZD three-month-old hiPSC neurons cultured with DMSO, Clozapine, Loxapine, Olanzapine, Risperidone and Thioridazine for the last three weeks of neuronal differentiation and sequentially transduced with LV-SYNP-HTG and Rabies-ENVAΔG-RFP. Only βIII-tubulin-positive events were counted. Error bars are SE. Asterisk used as follows: *** p<0.001.

FIG. 9. Additional controls validating observations of decreased neuronal connectivity in SCZD hiPSC neurons. FIG. 9A and FIG. 9B. Comparison of manual counts (FIG. 9A) of Rabies-ENVAΔG-RFP-labeled and LV-SYNP-HTG-labeled cells and integrated pixel density ratios (FIG. 9B) of Rabies-ENVAΔG-RFP-positive pixels to LV-SYNP-HTG-positive pixels show very similar results between control and SCZD hiPSC neurons. FIG. 9C. Histogram showing relative pixels of averaged control and SCZD hiPSC neurons, when cultured following sequential transduction with LV-SYNP-HTG and Rabies-ENVAΔG-RFP for 10 days. There was no significant difference in the pixel ratio between control and SCZD hiPSC neurons, but there was a significant decrease in the pixel ratios between control and SCZD hiPSC neurons. FIG. 9D. Histogram showing relative pixels of averaged control and SCZD hiPSC neurons, when cultured following sequential transduction with LV-SYNP-HTG and Rabies-ENVAΔG-RFP for 10 days. FIG. 9E. Histogram showing FACS analysis of control and SCZD three-month-old hiPSC neurons cultured on astrocytes and sequentially transduced with LV-TVA-H2BGFP and Rabies-ENVAΔG-RFP. Only βIII-tubulin-positive events were counted. Error bars are SE. Asterisks used as follows: * p<0.05, *** p<0.001.

FIG. 10. Dopaminergic TH-positive SCZD hiPSC neurons. FIG. 10A. Representative images showing TH-positive neurons in three-month-old hiPSC control and SCZD neural populations, costained with βIII-tubulin and DAPI. 200×, scale bar 80 μm. FIG. 10B. Representative images showing single TH-positive neurons in three-month-old hiPSC control and SCZD neural populations, costained with βIII-tubulin. Mature neurons are marked with a LV-SYNP-GFP reporter. 400×, scale bar 80 μm.

FIG. 11. Synaptic protein levels in control and SCZD hiPSC neurons. FIG. 11A. Representative images showing colocalization (indicated by white arrowheads) of VGLUT1-positive and PSD95-positive synaptic densities on neuronal dendrites. 2400×, scale bar 10 μm. FIG. 11B. Representative images showing colocalization (indicated by white arrowheads) of VGAT-positive and GEPH-positive synaptic densities on control and SCZD hiPSC neurons. 2400×, scale bar 10 μm. FIG. 11C. FACS analysis shows ˜30% of hiPSC NPC lines differentiate to GAD65/67+ cells. Error bars are SE. FIG. 11D. Representative images showing colocalization of GAD65/67 and βIII-tubulin in control and SCZD hiPSC neurons. 200×, scale bar 200 μm.

FIG. 12. Decreased neurites and synaptic protein levels in SCZD hiPSC neurons. FIG. 12A. Representative images of rare labeling of individual hiPSC neurons by low titer LV-SYNP-GFP. 800×, scale bar 20 μm. Neurites indicated by white arrows. FIG. 12B. Histogram showing decreased neurites in SCZD hiPSC neurons. FIG. 12C. Histogram showing a trend of decreased PSD95 synaptic density in SCZD hiPSC neurons. FIG. 12D-I. Histograms of synapse protein levels relative to MAP2AB for control and SCZD hiPSC neurons. Synaptic proteins assayed include SYN (FIG. 12D), VGLUT1 (FIG. 12E), GLUR1 (FIG. 12F), PSD95 (FIG. 12G), VGAT (FIG. 12H) and GEPH (FIG. 12I). Error bars are SE. Asterisks used as follows: *** p<0.001.

FIG. 13. Calcium transient analysis shows no difference in basal spontaneous activity between control and SCZD hiPSC neurons. FIG. 13A. Following incubation with the calcium binding dye Fluo-4AM, hiPSC neurons show spontaneous changes in Fluo-4AM fluorescence. Frames of a calcium time-lapse movie of a control hiPSC neuron culture at 0, 15, 30, 60 and 90 seconds. 13 ROIs with fluctuating calcium levels throughout movie are numbered. 100×, scale bar 400 μm. FIG. 13B. Calcium traces (FIG. 13B, left panel), plotting fluorescent intensity versus time, for individual ROIs in the movie shown above. One trace is shown per ROI. Spike events (FIG. 13B, middle panel) are automatically identified throughout 3,000 frames of a 90-second movie. The outline indicates spike events, which are identified based on the amplitude and slope (dF/F) of the calcium trace. Raster plots (FIG. 13B, right panel) of spike events occurring at each ROI over time. FIG. 13C. Histogram showing no difference between the spike amplitude of spontaneous calcium transients of control and SCZD hiPSC neurons. FIG. 13D. Histogram showing no difference between the total numbers of spontaneous calcium transients per total number of ROIs in cultures of control and SCZD hiPSC neurons. FIG. 13E. Representative analysis of synchronized and unsynchronized spontaneous calcium transient activity. Calcium traces (FIG. 13E, left panel), spike events (FIG. 13E, middle panel) and raster plots (FIG. 13E, right panel) of spike events occurring at each ROI over time are shown. FIG. 13F. Histogram showing no change in percentage synchronicity (total synchronized events per total events) between control and SCZD hiPSC neurons. FIG. 13G. Histogram showing no change in maximum percentage synchronicity (maximum number of ROIs involved in a synchronized event per total number of ROIs) between control and SCZD hiPSC neurons. Error bars are SE.

FIG. 14. Microarray gene analysis of control and SCZD hiPSC neurons. FIG. 14A. Heat map showing differential expression of 596 unique genes (271 upregulated and 325 downregulated) showing greater than 1.30-fold expression changes between SCZD and control hiPSC neurons. FIG. 14B. Principle component analysis of gene expression of three independent differentiations of hiPSC neurons from each of four control and four SCZD patients. FIG. 14C. qPCR validation of altered expression of GRIN2A, GRM7, DRD2, PDE4D, and LEF1, as well as response to three weeks of treatment with Loxapine (striped bars) in six-week-old hiPSC neurons. Asterisks used as follows: *** p<0.001.

FIG. 15. Genotyping of patients and gene expression analysis in hiPSC neurons. FIG. 15A. CNV analysis of SCZD patients. No CNVs in genes already implicated in SCZD or BD were identified in families 1 (patient 1) or 2 (patients 2 and 3). Patient 4 and 5 (family 3) showed numerous mutations, including deletion of the first exon of NRG3 isoform 2, deletions of CYP2C19 and GALNT11, and intergenic duplication of GABARB2-GABARA6. FIG. 15B. qPCR analysis for three candidate genes identified by CNV analysis reveals that genotype did not accurately predict gene expression changes in 1-month-old hiPSC neurons. FIG. 15C. CNV data showing the NRG3 deletion in patients 4 and 5 was likely inherited from the unaffected mother. FIG. 15D. qPCR reveals decreased NRG3 and increased NRG1 expression in 1-month-old hiPSC neurons in all patients relative to controls, irrespective of CNV status. Error bars are SE. Asterisks used as follows: * p<0.05, ** p<0.01, *** p<0.001.

FIG. 16. RNA expression analysis of SCZD hiPSC neurons, in untreated and Loxapine-treated conditions. Heat map showing differential expression of 3467 unique genes (1172 upregulated and 2295 downregulated) showing greater than 2.0-fold expression changes between SCZD and control hiPSC neurons.

FIG. 17. RNA expression analysis of SCZD hiPSC neurons, in untreated and Loxapine-treated conditions. FIG. 17A. GO analysis revealed the pathways most significantly affected in SCZDhiPSC neurons following treatment with Loxapine. Specifically, a number of genes implicated in cytoskeleton remodeling and signal transduction were identified. FIG. 17B. Heat maps showing microarray expression profiles of altered expression of a number of cytoskeleton remodeling genes.

FIG. 18. Increased rate of neural migration in SCZD hiPSC NPCs. FIG. 18A. Representative images of NPCs taken during a scratch migration assay. Brightfield and fluorescence images of lentiviral CAG-GFP transfected NPCs were taken every hour for up to 7 days—images shown were taken 0 hours and 100 hours post scratch. FIG. 18B. Histograms showing increasedmigration in SCZD hiPSC NPCs. Top histogram evaluates average speed by dividing the width of the initial scratch by the amount of time required to fill the gap. The bottom histogram calculates maximum speed of NPC migration by determining the rate of change of integrated pixel intensity within the scratch area over each five hour period and reporting the maximum rate. Error bars are SE. Asterisks used as follows: *** p<0.001.

FIG. 19. Altered cellular proliferation or cell cycle dynamics does not explain increased neural migration of SCZD hiPSC NPCs. FIG. 19A. Histogram showing no significant differences between the doubling time of control and SCZD hiPSC NPCs, when calculated by daily cell counts over a 7-day period. FIG. 19B. Histogram showing the cell cycle distribution of control and SCZD hiPSC NPC. There is no significant difference in the percentage of cells in G1, S or G2 between control and SCZD hiPSC NPC. Error bars are SD (FIG. 19A) and SE (FIG. 19B).

FIG. 20. Decreased WNT activity in SCZD hiPSC NPCs. Histogram showing decreased WNT reporter (TOPFLASH) activity relative to total protein content in SCZD hiPSC NPCs. Error bars are SE. Asterisks used as follows: ** p<0.01

### DETAILED DESCRIPTION OF THE INVENTION

### I. Definitions

The following definitions are provided to facilitate understanding of certain terms used frequently herein and are not meant to limit the scope of the present disclosure.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “protein” denotes an amino acid polymer or a set of two or more interacting or bound amino acid polymers.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion the gene is positioned between two transposon linker sequences that allow insertion into the host genome as well as subsequent excision.

The term “transfection” or “transfecting” is defined as a process of introducing nucleic acid molecules into a cell. The introduction may be accomplished by non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art.

A “cell culture” is a population of cells residing outside of an organism. These cells are optionally primary cells isolated from a cell bank, animal, or blood bank, or secondary cells that are derived from one of these sources and have been immortalized for long-lived in vitro cultures.

A “somatic cell” is a cell forming the body of an organism. Somatic cells include cells making up organs, skin, blood, bones and connective tissue in an organism, but not germline cells.

A “stem cell” is a cell characterized by the ability of self-renewal through mitotic cell division and the potential to differentiate into a tissue or an organ. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells.

“Pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

An “induced pluripotent stem cell” refers to a pluripotent stem cell artificially (e.g. non-naturally, in a laboratory setting) derived from a non-pluripotent cell. A “non-pluripotent cell” can be a cell of lesser potency to self-renew and differentiate than a pluripotent stem cell. Cells of lesser potency can be, but are not limited to adult stem cells, tissue specific progenitor cells, primary or secondary cells. An adult stem cell is an undifferentiated cell found throughout the body after embryonic development. Adult stem cells multiply by cell division to replenish dying cells and regenerate damaged tissue. Adult stem cells have the ability to divide and create another like cell and also divide and create a more differentiated cell. Even though adult stem cells are associated with the expression of pluripotency markers such as Rex1, Nanog, Oct4 or Sox2, they do not have the ability of pluripotent stem cells to differentiate into the cell types of all three germ layers. Adult stem cells have a limited potency to self renew and generate progeny of distinct cell types. Without limitation, an adult stem cell can be a hematopoietic stem cell, a cord blood stem cell, a mesenchymal stem cell, an epithelial stem cell, a skin stem cell or a neural stem cell. A tissue specific progenitor refers to a cell devoid of self-renewal potential that is committed to differentiate into a specific organ or tissue. A primary cell includes any cell of an adult or fetal organism apart from egg cells, sperm cells and stem cells. Examples of useful primary cells include, but are not limited to, skin cells, bone cells, blood cells, cells of internal organs and cells of connective tissue. A secondary cell is derived from a primary cell and has been immortalized for long-lived in vitro cell culture.

The term “reprogramming” refers to the process of dedifferentiating a non-pluripotent cell (e.g., an origin cell) into a cell exhibiting pluripotent stem cell characteristics (e.g., a human induced pluripotent stem cell).

Where appropriate the expanding transfected derived stem cell may be subjected to a process of selection. A process of selection may include a selection marker introduced into a an induced pluripotent stem cell upon transfection. A selection marker may be a gene encoding for a polypeptide with enzymatic activity. The enzymatic activity includes, but is not limited to, the activity of an acetyltransferase and a phosphotransferase. In some embodiments, the enzymatic activity of the selection marker is the activity of a phosphotransferase. The enzymatic activity of a selection marker may confer to a transfected induced pluripotent stem cell the ability to expand in the presence of a toxin. Such a toxin typically inhibits cell expansion and/or causes cell death. Examples of such toxins include, but are not limited to, hygromycin, neomycin, puromycin and gentamycin. In some embodiments, the toxin is hygromycin. Through the enzymatic activity of a selection maker a toxin may be converted to a non-toxin, which no longer inhibits expansion and causes cell death of a transfected induced pluripotent stem cell. Upon exposure to a toxin a cell lacking a selection marker may be eliminated and thereby precluded from expansion.

Identification of the induced pluripotent stem cell may include, but is not limited to the evaluation of the afore mentioned pluripotent stem cell characteristics. Such pluripotent stem cell characteristics include without further limitation, the expression or non-expression of certain combinations of molecular markers. Further, cell morphologies associated with pluripotent stem cells are also pluripotent stem cell characteristics.

The term “hiPSC-derived neural cell” refers to a neural progenitor cell (NPC) or a mature neuron that has been derived (e.g., differentiated) from a hiPSC cell in vitro. The hiPSCs can be differentiated by any appropriate method known in the art (e.g., Marchetto, M. C. et al., Cell Stem Cell, 3, 649-657 (2008); Yeo, G. W. et al., PLoS Comput Biol, 3, 1951-1967 (2007)).

A neural progenitor is a cell that has a tendency to differentiate into a neural cell and does not have the pluripotent potential of a stem cell. A neural progenitor is a cell that is committed to the neural lineage and is characterized by expressing one or more marker genes that are specific for the neural lineage. Examples of neural lineage marker genes are N-CAM, the intermediate-filament protein nestin, SOX2, vimentin, A2B5, and the transcription factor PAX-6 for early stage neural markers (i.e. neural progenitors); NF-M, MAP-2AB, synaptosin, glutamic acid decarboxylase, βIII-tubulin and tyrosine hydroxylase for later stage neural markers (i.e. differentiated neural cells). Neural differentiation may be performed in the absence or presence of co-cultured astrocytes.

The term “schizophrenia marker function” means any appropriate genetic or physiological (phenotypic) criteria that is more prevalent and/or pronounced in cells obtained or derived from a schizophrenic subject than in cells obtained or derived from a subject without schizophrenia.

For specific proteins described herein (e.g., Sox2, KLF4, cMYC), the named protein includes any of the protein's naturally occurring forms, or variants that maintain the protein transcription factor activity (e.g., within at least 50%, 80%, 90%, 95%, 96%, 97%, 98%, 99% or 100% activity compared to the native protein). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference or functional fragment thereof.

As used herein, the terms “prevent” and “treat” are not intended to be absolute terms. Treatment can refer to any delay in onset or prevention, e.g., reduction in the frequency or severity of symptoms, amelioration of symptoms, improvement in patient comfort, reduction in skin inflammation, and the like. The effect of treatment can be compared to an individual or pool of individuals not receiving a given treatment, or to the same patient before, or after cessation of, treatment.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

The “subject” as used herein is a subject in need of treatment for schizophrenia. The subject is preferably a mammal and is most preferably a human, but also may include laboratory, pet, domestic, or livestock animals.

The term “disease” refers to any deviation from the normal health of a mammal and includes a state when disease symptoms are present, as well as conditions in which a deviation (e.g., infection, gene mutation, genetic defect, etc.) has occurred, but symptoms are not yet manifested. According to the present invention, the methods disclosed herein are suitable for use in a patient that is a member of the Vertebrate class, Mammalia, including, without limitation, primates, livestock and domestic pets (e.g., a companion animal). Typically, a patient will be a human patient.

As used herein, “administering” means any appropriate method of providing a treatment to a patient such as oral (“po”) administration, administration as a suppository, topical contact, intravenous (“iv”), intraperitoneal (“ip”), intramuscular (“im”), intralesional, intranasal or subcutaneous (“sc”) administration, or the implantation of a slow-release device e.g., a mini-osmotic pump or erodible implant, to a subject. Administration is by any appropriate route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (i.e., other than through the alimentary tract, such as intramuscular, intravenous, intra-arterial, transdermal and subcutaneous) administration.

As used herein, “increase,” or “increasing” in reference to a treated cell means an increase in a measured parameter (e.g., activity, expression, signal transduction, neuron degeneration) in a treated cell (tissue or subject) in comparison to an untreated cell (tissue or subject). A comparison can also be made of the same cell or tissue or subject between before and after treatment. The increase is sufficient to be detectable. In some embodiments, the increase in the treated cell is at least about 10%, 20%, 30%, 40%, 50%, 60%, 70%, 80%, 90%, 1-fold, 2-fold, 3-fold, 4-fold or more in comparison to an untreated cell.

As used herein, “inhibit,” “prevent”, “reduce,” “inhibiting,” “preventing” or “reducing” in reference to a treated cell are used interchangeably herein. These terms refer to the decrease in a measured parameter (e.g., activity, expression, signal transduction, neuron degeneration) in a treated cell (tissue or subject) in comparison to an untreated cell (tissue or subject). A comparison can also be made of the same cell or tissue or subject between before and after treatment. The decrease is sufficient to be detectable. In some embodiments, the decrease in the treated cell is at least about 10%, 20%, 30%, 40%, 50%, 60%, 70%, 80%, 90%, or completely inhibited in comparison to an untreated cell. In some embodiments the measured parameter is undetectable (i.e., completely inhibited) in the treated cell in comparison to the untreated cell.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

The terms “effective amount,” “therapeutically effective amount” or “pharmaceutically effective amount” as used herein refers to that amount of the therapeutic agent sufficient to ameliorate one or more aspects of the disorder (e.g., schizophrenia). The result can be reduction and/or alleviation of the signs, symptoms, or causes of a disease, or any other desired alteration of a biological system. For example, an “effective amount” for therapeutic uses is the amount of a composition required to provide a clinically significant decrease in schizophrenia. For example, for the given aspect (e.g., length of incidence), a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. An appropriate “effective” amount in any individual case may be determined using techniques, such as a dose escalation study.

The term “schizophrenic” refers to a subject that has been clinically diagnosed with schizophrenia, displays one or more schizophrenia symptoms or has a family history of schizophrenia. A subject having a family history of schizophrenia is also referred to herein as a “pre-symptomatic” subject or “pre-symptomatic schizophrenic.” A pre-symptomatic subject is a subject that has not developed schizophrenia symptoms yet. A pre-symptomatic subject may be a subject having a family history of schizophrenia (i.e. a genetic schizophrenic). Non-limiting examples of schizophrenia symptoms include physiologic symptoms (e.g., auditory hallucinations, paranoia, delusions, disorganized speech and thinking, psychomotor agitation, depression; see also Diagnostic and Statistical Manual of Mental Disorders, Fourth Edition, 2000, American Psychiatric Association, Washington, D.C. (“DSM-IV”) and genetic symptoms (e.g., aberrant gene expression of one or more genes associated with schizophrenia. Non-limiting examples of genes associated with schizophrenia are listed in Table 5 (e.g., AQP4, HEY2, GRIK1, GFAP, SPP1, SPARCL1). In some embodiments, a schizophrenic subject is a subject not clinically diagnosed with schizophrenia. In other embodiments, a schizophrenic subject is a subject displaying one or more schizophrenia symptoms. In other embodiments, a schizophrenic subject is a subject having a family history of schizophrenia. In other embodiments, a schizophrenic subject is a pre-symptomatic subject (i.e. a pre-symptomatic schizophrenic).

### II. Methods

The methods according to the embodiments provided herein inter alia, are useful in the area of schizophrenia drug development, diagnosis and personalized medicine.

A. Methods of Screening for Schizophrenia Compounds

In one aspect, a method of determining whether a test compound is capable of improving a schizophrenia marker function in a hiPSC-derived neural cell is provided. The method includes contacting a test compound with a hiPSC-derived neural cell derived from a schizophrenic subject. The hiPSC-derived neural cell exhibits a schizophrenia marker function at a first level in the absence of the test compound. Then, a second level of the schizophrenia marker function is determined in the presence of the test compound. The second level is compared to a control level. A smaller difference between the second level and the control level than between the first level and the control level indicates that the test compound is capable of improving the schizophrenia marker function. A control level of a schizophrenia marker function as provided herein refers to a level of the schizophrenia marker function in a control cell. A control cell is a cell that is derived from a non-schizophrenic (e.g., healthy) subject or a pre-symptomatic subject. In some embodiments, the control level is a level lower than the first level. In other embodiments, the control level is a level higher than the first level. In some embodiments, the control level of a schizophrenia marker function is a level of the schizophrenia marker function in a healthy subject. In other embodiments, the control level of a schizophrenia marker function is a level of the schizophrenia marker function in a pre-symptomatic subject. In some embodiments, the smaller difference indicates that the schizophrenic subject is responsive to the test compound. Thus, where the subject is not responsive to the test compound the difference between the second level of a schizophrenia marker function and the control level is bigger than the difference between the first level and the control level.

In one embodiment, a smaller difference indicates that the schizophrenic subject is responsive to the test compound. A subject is “responsive” when the subject experiences a reduction in one or more schizophrenic symptoms. In this way, the screening methods can be used to assess the efficacy of various test compounds on neural cells modeled from the subject's own cells in vitro, thereby identifying treatment regimens that may be most effective to the particular subject to be treated without subjecting the subject to multiple experimental treatment regimens, the side effects accompanying any particular treatment regimen, as well as side effects associated with beginning a new treatment regimen and changing treatment regimens.

Accordingly, in one embodiment, where the test compound is found to be capable of improving a schizophrenia marker function in a hiPSC-derived neural cell, the method further comprises administering an effective amount of the test compound (e.g., a test compound identified as described above) to the schizophrenic subject in need of treatment for schizophrenia (e.g., the subject from whom the hiPSCs were derived for the screening method).

In the methods provided herein, a test compound may be contacted with a hiPSC-derived neural cell and/or administered to a subject in need of treatment for schizophrenia. In some embodiments, the compound may be a known compound such as typical antipsychotics, atypical antipsychotics, or combinations thereof. In other embodiments, the test compound is clozapine, loxapine, olanzapine, risperidone, thioridazine, perphenazine, aripiprazole, iloperidone, ziprasidone, paliperidone, lurasidone, molindone, asenapine, mesoridazine, quetiapine, or trifluoperazine. In other embodiments, the compound is clozapine, loxapine, olanzapine, risperidone, and thioridazine. In some embodiments, the compound is loxapine. In another embodiment, the compound is not currently approved for the indication of schizophrenia.

The methods provided herein may be used for identifying test compounds that may be useful for treating schizophrenia, for identifying test compounds that are currently marketed for other psychotic or non-psychotic indications but may also be useful for treating schizophrenia, and/or for identifying compounds that are currently marketed for schizophrenia that may be useful for treating schizophrenia in a particular subject (e.g., personalized medicine applications).

### Generation of Human Induced Pluripotent Stem Cell-Derived Neural Cells

The methods described herein employ a hiPSC-derived neural cell. In any of the methods, the hiPSC-derived neural cell can be produced by: a) obtaining a primary cell from a control subject (i.e., non-schizophrenic) and/or a schizophrenic subject, b) reprogramming the primary cell to form a hiPSC (e.g., through viral transfection), and c) allowing the hiPSC to differentiate and/or promoting differentiation of the hiPSC in vitro to form a hiPSC-derived neural cell. The primary cell can be any somatic cell. In some embodiments, the primary cell is a fibroblast cell. In some embodiments, the primary cell is obtained from a schizophrenic subject. In some embodiments, the method includes (i) reprogramming a fibroblast cell thereby forming a fibroblast-derived hiPSC; and (ii) differentiating the fibroblast-derived hiPSC thereby forming the hiPSC-derived neural cell. Differentiating the fibroblast-derived hiPSC may include expansion of fibroblast cell after transfection, optional selection of transfected cells and identification of resulting pluripotent stem cells. Expansion as used herein includes the production of progeny cells by a transfected fibroblast cell under conditions well know in the art (Soldner, F. et al. Cell 136:964-977 (2009); Yamanaka, S. Cell 137:13-17 (2009)). Expansion may occur in the presence of suitable media and cellular growth factors. Cellular growth factors are agents which cause cells to migrate, differentiate, transform or mature and divide. Cellular growth factors are polypeptides which can usually be isolated from various normal and malignant mammalian cell types. Some growth factors can also be produced by genetically engineered microorganisms, such as bacteria (E. coli) and yeasts. Cellular growth factors may be supplemented to the media and/or may be provided through co-culture with irradiated embryonic fibroblast that secrete such cellular growth factors. Examples of cellular growth factors include, but are not limited to, FGF, bFGF2, and EGF.

Where appropriate the expanding fibroblast cell may be subjected to a process of selection. A process of selection may include a selection marker introduced into a fibroblast cell upon transfection. A selection marker may be a gene encoding for a polypeptide with enzymatic activity. The enzymatic activity includes, but is not limited to, the activity of an acetyltransferase and a phosphotransferase. In some embodiments, the enzymatic activity of the selection marker is the activity of a phosphotransferase. The enzymatic activity of a selection marker may confer to a transfected neural stem cell the ability to expand in the presence of a toxin. Such a toxin typically inhibits cell expansion and/or causes cell death. Examples of such toxins include, but are not limited to, hygromycin, neomycin, puromycin and gentamycin. In some embodiments, the toxin is hygromycin. Through the enzymatic activity of a selection maker a toxin may be converted to a non-toxin which no longer inhibits expansion and causes cell death of a transfected neural stem cell. Upon exposure to a toxin a cell lacking a selection marker may be eliminated and thereby precluded from expansion.

Identification of the hiPSC may include, but is not limited to the evaluation of the afore mentioned pluripotent stem cell characteristics. Such pluripotent stem cell characteristics include without further limitation, the expression or non-expression of certain combinations of molecular markers. Further, cell morphologies associated with pluripotent stem cells are also pluripotent stem cell characteristics.

In some embodiments, the hiPSC exhibits normal expression of endogenous pluripotency genes. In another embodiment, the hiPSC represses viral genes. In another embodiment, the hiPSC both exhibits normal expression of endogenous pluripotency genes and repression of viral genes.

### Schizophrenia Marker Functions

The hiPSC-derived neural cells as described above may be used in any of the methods disclosed herein as appropriate. The methods described herein may also include assessing or measuring one or more schizophrenia marker functions. In one embodiment, the schizophrenia marker function is a level of gene expression. As provided herein, any appropriate genetic or physiological (e.g., phenotypic) criteria that is more prevalent and/or pronounced in cells obtained or derived from a schizophrenic subject than in cells obtained or derived from a non-schizophrenic subject is a schizophrenia marker function. In one embodiment, the schizophrenia marker function is a level of protein production. In another embodiment, the schizophrenia marker function is a structural characteristic of a neural cell. In another embodiment, the schizophrenia marker function is a characteristic of intracellular relation/communication. In some embodiments, the schizophrenia marker function is a number of neurites extending from the hiPSC-derived neural cell, a level of synaptic proteins expressed by the hiPSC-derived neural cell, a level of PSD95 expressed by the hiPSC-derived neural cell, a level of synaptic density of the hiPSC-derived neural cell, a level of neural connectivity of the hiPSC-derived neural cell, a level of synaptic plasticity of the hiPSC-derived neural cell, a level of NRG1 expressed by the hiPSC-derived neural cell, a level of a glutamate receptor expressed by the hiPSC-derived neural cell, a level of a neuregulin pathway component expressed by the hiPSC-derived neural cell, a level of a synaptic protein expressed by the hiPSC-derived neural cell, a level of a cAMP component expressed by the hiPSC-derived neural cell, a level of a calcium signaling pathway component expressed by the hiPSC-derived neural cell, a level of a Wnt signaling pathway component expressed by the hiPSC-derived neural cell, a level of a Notch growth factor expressed by the hiPSC-derived neural cell, a level of neural migration by the hiPSC-derived neural cell or a level of a cell adhesion component by the hiPSC-derived neural cell. In other embodiments, the schizophrenia marker function is a number of neurites extending from the hiPSC-derived neural cell, a level of synaptic proteins expressed by the hiPSC-derived neural cell, a level of PSD95 expressed by the hiPSC-derived neural cell, a level of synaptic density of the hiPSC-derived neural cell, a level of neural connectivity of the hiPSC-derived neural cell, a level of synaptic plasticity of the hiPSC-derived neural cell, a level of NRG1 expressed by the hiPSC-derived neural cell, a level of a glutamate receptor expressed by the hiPSC-derived neural cell, a level of a neuregulin pathway component expressed by the hiPSC-derived neural cell, a level of a synaptic protein expressed by the hiPSC-derived neural cell, a level of a cAMP component expressed by the hiPSC-derived neural cell, a level of a calcium signaling pathway component expressed by the hiPSC-derived neural cell, a level of a Wnt signaling pathway component expressed by the hiPSC-derived neural cell, a level of a Notch growth factor expressed by the hiPSC-derived neural cell, a level of neural migration by the hiPSC-derived neural cell and a level of a cell adhesion component by the hiPSC-derived neural cell.

When the schizophrenia marker function is the number of neurites extending from the hiPSC-derived neural cell, a decrease in the number of neurites is indicative of an increased likelihood and/or severity of schizophrenia.

Synaptic density can be measured, for example, by identifying colocalized synaptic puncta of VGLUT1 and PSD95, thresholding on size and then manually counting large colocalized puncta along a given length of neurite. Decreased synaptic density is indicative of an increased likelihood and/or severity of schizophrenia.

Neural connectivity can be measured by, for example, using trans-synaptic labeling using tracers (e.g., rabies viral trans-synaptic labeling). Decreased trans-synaptic labeling is indicative of an increased likelihood and/or severity of schizophrenia.

Synaptic plasticity can be measured by, for example, measuring variations in intracellular calcium levels.

For gene expression levels (e.g., NRG3, NRG2, NRG1, PSD95, and PSD93), a decrease in expression is indicative of an increased likelihood and/or severity of schizophrenia.

Glutamate receptors are synaptic receptors located primarily on the membranes of neural cells. They include ionotropic (e.g., AMPA, Kainate, and NMDA families) and metabotropic (e.g., groups 1, 2, and 3) receptors.

The neuregulins are a family of structurally-related proteins that are part of the EGF family of proteins. Neuregulin pathway components include the neuregulins themselves (e.g., NRG1 and any of its isoforms, NRG2, NRG3, or NRG4) as well as proteins that interact with the neuregulins and nucleic acids that encode either the neuregulins or their associated proteins. Exemplary neuregulin pathway components include, but are not limited to ERBB2, ERBB3, ERBB4, and LIMK1.

A synaptic protein is any appropriate protein that affects synaptic transmission. In particular, synaptic proteins include regulators of synaptic transmission are palmitoylated proteins that are concentrated at pre- or postsynaptic sites. On the presynaptic side, palmitoylated proteins regulate synaptic vesicle fusion and neurotransmitter synthesis and release. These include several members of the synaptotagmin family, synaptobrevin 2 and SNAP25 (synaptosomal-associated protein, 25 kDa), and GAD65 (glutamic acid decarboxylase, 65 kDa), which synthesizes the inhibitory neurotransmitter GABA (-aminobutyric acid). Palmitoylated presynaptic proteins also include the 2A-subunit of the voltage-dependent calcium channel, and the -subunit of sodium channels. GAP43 (growth-associated protein 43), paralemmin and NCAM140 (neural cell-adhesion molecule) are palmitoylated proteins that are associated with axonal growth cones. RhoB (Ras homologue B) and Tc10 are small GTPases that regulate cytoskeletal dynamics. On the postsynaptic side, many receptors are palmitoylated, including G-protein-coupled receptors (GPCRs), the METABOTROPIC glutamate receptor subunit mGluR4 and the kainate receptor subunit GluR6. Numerous downstream signaling enzymes are also palmitoylated, including the G-protein-subunit, Fyn (a member of the Src family of non-receptor tyrosine kinases) and a Ras small GTPase. By scaffolding receptors and enzymes, palmitoylated PDZ proteins have an important role in the assembly of postsynaptic signaling pathways. Palmitoylated PDZ proteins at the synapse include the postsynaptic density proteins PSD95 and PSD93, which bind the tails of NMDA (N-methyl-d-asparate) receptors and the AMPA (-amino-3-hydroxy-5-methyl-4-isoxazole propionic acid)-receptor-associated protein stargazin, and GRIP1b (glutamate-receptor-interacting protein 1b) and ABP-L (AMPA-receptor-binding protein-L), which bind the tail of the AMPA receptor subunit GluR2. Alaa El-Din El-Husseini & David S. Bredt. “Protein palmitoylation: a regulator of neural development and function” Nature Reviews Neuroscience 3, 791-802 (2002).

cAMP is derived from adenosine triphosphate (ATP) and used for intracellular signal transduction in many different organisms, conveying the cAMP-dependent pathway. Exemplary cAMP components include, but are not limited to, protein kinase A (PKA), exchange proteins (e.g., Epac1, Epac2), Rap1, epinephrine (adrenaline), G protein, adenylyl cyclase, cAMP receptor protein (CRP, CAP), as well as the lac operon.

Calcium signaling pathway components affect the influx of calcium resulting from activation of ion channels or by indirect signal transduction pathways. Exemplary calcium signaling pathway components include, but are not limited to, phospholipase C (PLC), G-protein couple receptors, PIP2, IP3, IP3 receptor, diacylglycerol (DAG), protein kinase C, “Store Operated Channels” (SOCs), Orai1, STIM1 phospholipase A2 beta, nicotinic acid adenine dinucleotide phosphate (NAADP), STIM 1, calmodulin, and calcium-calmodulin dependent protein kinases.

Exemplary Wnt signaling pathway components include, but are not limited to, WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11, and WNT16, as well as cell-surface receptors of the Frizzled family, Dishevelled family proteins, β-catenin, axin, GSK-3, protein APC, as well as TCF/LEF family transcription factors.

Members of the notch signaling pathway include, but are not limited to, NOTCH1, NOTCH2, NOTCH3, and NOTCH4 as well as notch ligands.

Neural cells reside in different areas of the brain during their development. For instance, neural progenitors reside in the developing neocortex and upon development migrate to different areas of the cortex depending on their function. Surprisingly, Applicants have found that schizophrenic neural precursors exhibit an impaired neural migration. The level of neural migration of hiPSC-derived neural cells obtained or derived from a schizophrenic subject (i.e. schizophrenic neural migration) may be higher than the level of neural migration of hiPSC-derived neural cells derived or obtained from a non-schizophrenic subject (i.e. non-schizophrenic neural migration). In some embodiments, the level of schizophrenic neural migration is higher than the level of non-schizophrenic neural migration. When the schizophrenia marker function is a level of neural migration by the hiPSC-derived neural cell, an increase in the level of neural migration is indicative of an increased likelihood and/or severity of schizophrenia.

In some embodiments, the level of a cell adhesion component expressed by the hiPSC-derived neural cell is decreased. When the schizophrenia marker function is a level of a cell adhesion component expressed by the hiPSC-derived neural cell, a decrease in the level of a cell adhesion component expressed by the hiPSC-derived neural cell is indicative of an increased likelihood and/or severity of schizophrenia.

Any one or more of these schizophrenia marker functions can be used in any of the following methods. In another embodiment, a schizophrenia marker function can first be identified by the method of identifying a schizophrenia marker function, described in detail below, and then employed in the methods of screening test compounds and/or methods of diagnosing schizophrenia.

B. Methods of Determining Whether a Subject is Schizophrenic

In one aspect, a method of determining whether a subject is schizophrenic is provided. The method includes determining a level of a schizophrenia marker function in a hiPSC-derived neural cell derived from a subject (a test subject) and comparing the level to a control level. A difference between the determined level and the control level indicates that the subject is schizophrenic. In some embodiments, the method further includes the steps of quantitating the level of the schizophrenia marker function to determine a test quantity, and comparing the test quantity to a control quantity to determine the severity of the subject's schizophrenia. Quantitating the level of the schizophrenia marker function may include quantification of any of the above described schizophrenia marker functions using methods well known in the art (e.g., quantifying the number of neurites of a hiPSC-derived neural cell, measuring synaptic density by quantifying colocalized synaptic puncta of VGLUT1 and PSD95 along a given length of neurite, measuring neural connectivity using trans-synaptic labeling using tracers (e.g., rabies viral trans-synaptic labeling), measuring gene expression levels (e.g., NRG3, NRG2, NRG1, PSD95, and PSD93)). A control quantity refers to the quantitated level of the schizophrenia marker function in a non-schizophrenic cell (e.g., healthy cell). For example, where the schizophrenia marker function is the level of neural connectivity, trans-synaptic labeling as described in the Example section may be used to measure (i) a test quantity of neural connectivity in hiPSC-derived neural cells from a schizophrenic subject and (ii) compare the test quantity to a control quantity which is a quantity of neural connectivity measured in hiPSC-derived neural cells from a non-schizophrenic subject.

In some embodiments, the method further includes the preliminary steps of creating a hiPSC-derived neural cell. As described above, hiPSC-derived neural cells can be made by obtaining a cell (e.g., a fibroblast cell) from the subject. In some embodiments, the cell is a fibroblast cell. The cell is then reprogrammed to form a hiPSC. Then the hiPSC is allowed to differentiate thereby forming a hiPSC-derived neural cell.

In some embodiments, the method further includes treating the subject in need of treatment for schizophrenia. Treating the subject in need of treatment for schizophrenia includes administering to the subject an effective amount of a compound identified using any of the methods provided herein. The compound may be part of a powder, tablet, capsule, liquid, ointment, cream, gel, hydrogel, aerosol, spray, micelle, liposome or any other pharmaceutically acceptable form. One of ordinary skill in the art would readily appreciate that an appropriate vehicle for use with the compounds identified using the methods provided herein should be one that is well tolerated by a recipient of the composition. The vehicle should also readily enable the delivery of the compounds to appropriate target receptors. For example, one of ordinary skill in the art would know to consult Pharmaceutical Dosage Forms and Drug Delivery Systems, by Ansel, et al., Lippincott Williams & Wilkins Publishers; 7th ed. (1999) or a similar text for guidance regarding such formulations. The composition identified using the methods provided herein may be used in a number of ways. For instance, systemic administration may be required in which case the compounds can be formulated into a composition that can be ingested orally in the form of a tablet, capsule or liquid. Alternatively the composition may be administered by injection into the blood stream. Injections may be intravenous (bolus or infusion) or subcutaneous (bolus or infusion). The disclosed compounds can also be administered centrally by means of intracerebral, intracerebroventricular, or intrathecal delivery.

It will be readily appreciated that the amount of a compound required is determined by biological activity and bioavailability which in turn depends on the mode of administration, the physicochemical properties of the compound employed and whether the compound is being used as a monotherapy or in a combined therapy. For combined therapy the compound may be administered in combination with another pharmacological agent, for example, lithium, valproate, or an antidepressant, for example, fluoxetine. The frequency of administration will also be influenced by the above mentioned factors and particularly the half-life of the compound within the subject being treated. One of ordinary skill in the art would appreciate that specific formulations of compositions and precise therapeutic regimes (such as daily doses of the compounds and the frequency of administration) can be determined using known procedures. Such procedures conventionally employed by the pharmaceutical industry include in vivo experimentation and clinical trials.

The methods provided herein may be useful for diagnostic assessments for both symptomatic and asymptomatic (e.g., pre-symptomatic) subjects. As described above a subject is schizophrenic if the subject displays one or more schizophrenic symptoms. A subject that has not been diagnosed with schizophrenia (e.g., pre-symptomatic) but has a family history of schizophrenia is referred to as a schizophrenic subject. The methods may also be used in the area of personalized medicine. For instance, where a subject is in need of treatment for schizophrenia, the most effective and compliant drug specific for this subject can be determined in vitro using the methods provided herein. Without administering the drug to the subject it can be determined whether a drug is appropriate (i.e. effective, causing least side effects) for treatment of schizophrenia in a particular subject. Rather than administering different drugs to a subject sequentially to identify the most effective drug for that particular subject, the methods provided herein allow for simultaneous testing of a plurality of drugs in vitro. Therefore the subject is not subjected to multiple experimental treatment regimens, the side effects accompanying any particular treatment regimen, as well as side effects associated with beginning a new treatment regimen and changing treatment regimens. Further, the progression of a disease state and/or the efficacy of a treatment regimen can be assessed in a single subject in a recurrent, e.g., periodic, manner.

C. Methods of Identifying a Schizophrenia Marker Function

The methods provided herein, inter alia, are useful for identifying new schizophrenia marker functions. In one aspect, a method of identifying a schizophrenia marker function is provided. The method includes obtaining a cell from a schizophrenic subject and reprogramming the cell thereby forming a hiPSC. The hiPSC is allowed to differentiate thereby forming a hiPSC-derived neural cell derived from the schizophrenic subject. A level of a function of the hiPSC-derived neural cell is determined and the level is compared to a control level. A difference between the level and the control level indicates the function is a schizophrenia marker function. The level of a function of a hiPSC-derived neural cell includes any genetic or physiological criteria that is more prevalent and/or pronounced in a neural cell obtained or derived from a schizophrenic subject than in a neural cell obtained or derived from a non-schizphrenic subject. In some embodiments, the cell is a fibroblast cell. The method may include comparisons between: a) a single schizophrenic subject and a single non-schizophrenic subject, b) an average level exhibited by multiple schizophrenic subjects and an average level exhibited by multiple control subjects, or c) a pre-schizophrenic subject and the same subject after the manifestation of schizophrenic symptoms.

D. Methods of Determining Loxapine Marker Functions

Loxapine is a antipsychotic drug, which is primarily used for the treatment of schizophrenia. Loxapine is a dibenzazepine derivative and refers, in the customary sense, to CAS Registry No. 1977-10-2. The methods provided herein can be used to determine whether a subject is responsive to loxapine and whether loxapine is the most effective drug to treat schizophrenia in a particular subject. A loxapine compound as referred to herein is any compound having the same pharmacological properties as loxapine. Examples of loxapine compounds include pharmaceutically acceptable salts of loxapine or any derivatives of loxapine having the same pharmacological properties as loxapine.

In one aspect, a method of determining whether a schizophrenic subject is responsive to treatment with a loxapine compound is provided. The method includes contacting a loxapine compound with a hiPSC-derived neural cell. The hiPSC-derived neural cell is derived from the schizophrenic subject, and the hiPSC-derived neural cell exhibits a loxapine marker function at a first level in the absence of a loxapine compound. Then a second level of the loxapine marker function is determined and the second level is compared to a control level. A smaller difference between the second level and the control level than between the first level and the control level indicates the schizophrenic subject is responsive to treatment with a loxapine compound. The control level is the level of a loxapine marker function of a control cell. A control cell is a cell that is derived from a non-schizophrenic (e.g., healthy) subject or a pre-symptomatic subject. In some embodiments, the control level is a level lower than the first level. In other embodiments, the control level is a level higher than the first level. In some embodiments, the control level of a loxapine marker function is a level of the loxapine marker function in a healthy subject. In other embodiments, the control level of a loxapine marker function is a level of the loxapine marker function in a pre-symptomatic subject. In some embodiments, the smaller difference indicates that the schizophrenic subject is responsive to the loxapine compound. Thus, where the subject is not responsive to the loxapine compound the difference between the second level of a loxapine marker function and the control level is bigger than the difference between the first level and the control level.

A “loxapine marker function” is a schizophrenia marker function that is modified by treatment with a loxapine compound. In some embodiments, the loxapine marker function is a level of a cytoskeleton remodeling component expressed by the hiPSC-derived neural cell, a level of TGF signaling pathway component expressed by the hiPSC-derived neural cell, a level of NRG1 expressed by the hiPSC-derived neural cell, a level of a glutamate receptor expressed by the hiPSC-derived neural cell, a level of neural connectivity of the hiPSC-derived neural cell, or a level of a cell adhesion component expressed by the hiPSC-derived neural cell. In some embodiments, the loxapine marker function is a level of a cytoskeleton remodeling component expressed by the hiPSC-derived neural cell, a level of TGF signaling pathway component expressed by the hiPSC-derived neural cell, a level of NRG1 expressed by the hiPSC-derived neural cell, a level of a glutamate receptor expressed by the hiPSC-derived neural cell, a level of neural connectivity of the hiPSC-derived neural cell, and a level of a cell adhesion component expressed by the hiPSC-derived neural cell.

In some embodiments, the method further includes administering an effective amount of a loxapine compound to the schizophrenic subject in need of treatment for schizophrenia. In some embodiments, the hiPSC-derived neural cell is made by a method including reprogramming a fibroblast cell thereby forming a fibroblast-derived hiPSC and differentiating the fibroblast-derived hiPSC thereby forming the hiPSC-derived neural cell.

In another aspect, a method of determining whether a test compound is capable of improving a loxapine marker function is provided. The method includes contacting a test compound with a hiPSC-derived neural cell. The hiPSC-derived neural cell is derived from a schizophrenic subject, and the hiPSC-derived neural cell exhibits a loxapine marker function at a first level in the absence of the test compound. Then a second level of the loxapine marker function determined and the second level is compared to a control level. A smaller difference between the second level and the control level than between the first level and the control level indicates the test compound is capable of improving the loxapine marker function. In some embodiments, the smaller difference indicates the schizophrenic subject is responsive to the test compound. In some embodiments, the loxapine marker function is a level of a cytoskeleton remodeling component expressed by the hiPSC-derived neural cell, a level of TGF signaling pathway component expressed by the hiPSC-derived neural cell, a level of NRG1 expressed by the hiPSC-derived neural cell, a level of a glutamate receptor expressed by the hiPSC-derived neural cell, a level of neural connectivity of the hiPSC-derived neural cell, or a level of a cell adhesion component expressed by the hiPSC-derived neural cell. In other embodiments, the loxapine marker function is a level of a cytoskeleton remodeling component expressed by the hiPSC-derived neural cell, a level of TGF signaling pathway component expressed by the hiPSC-derived neural cell, a level of NRG1 expressed by the hiPSC-derived neural cell, a level of a glutamate receptor expressed by the hiPSC-derived neural cell, a level of neural connectivity of the hiPSC-derived neural cell, and a level of a cell adhesion component expressed by the hiPSC-derived neural cell.

In other embodiments, the method further includes administering an effective amount of the test compound to the schizophrenic subject in need of treatment for schizophrenia. Methods of treating schizophrenia applicable to the compounds identified through the methods disclosed herein are described in section C.

In some embodiments, the hiPSC-derived neural cell is made by a method including reprogramming a fibroblast cell thereby forming a fibroblast-derived hiPSC and differentiating the fibroblast-derived hiPSC thereby forming the hiPSC-derived neural cell. In some embodiments, the fibroblast cell is obtained from a schizophrenic subject. In some further embodiments, the schizophrenic subject is a pre-symptomatic subject.

### III. Examples

### A. Methodologies

Reprogramming hiPSCs

Control and SCZD HFs were obtained from cell repositories and were reprogrammed with tetracycline-inducible lentiviruses expressing the transcription factors OCT4, SOX2, KLF4, cMYC and LIN28⁷. Lentiviruses were packaged in 293T HEK cells transfected with Polyethylenimine (PEI) (Polysciences). HFs were transduced and then split onto mouse embryonic fibroblasts (mEFs). Cells were switched to HUES media (KO-DMEM (Invitrogen), 10% KO-Serum Replacement (Invitrogen), 10% Plasminate (Talecris), 1× Glutamax (Invitrogen), 1×NEAA (Invitrogen), 1×2 βmercaptoethanol (Sigma) and 20 ng/ml FGF2 (Invitrogen)) and 1 μg/ml Doxycycline (Sigma) was added to HUES media for the first 21-28 days of reprogramming. hiPSCs were generally grown in HUES media: early passage hiPSCs were split through manual passaging, while at higher passages hiPSCs could be enzymatically passaged with 1 mg/ml Collagenase (Sigma).

hiPSC Differentiation to NPCs and Neurons

Embryoid bodies were generated from hiPSCs and then transferred to nonadherent plates (Corning). Colonies were maintained in suspension in N2 media (DMEM/F12 (Invitrogen), 1×N2 (Invitrogen)) for 7 days and then plated onto polyornithine (PORN)/Laminin-coated plates. Visible rosettes formed within 1 week and were manually dissected and cultured in NPC media (DMEM/F12, 1×N2, 1×B27-RA (Invitrogen), 1 μg/ml Laminin (Invitrogen) and 20 ng/ml FGF2 (Invitrogen). NPCs are maintained at high density, grown on PORN/Laminin-coated plates in NPC media and split approximately 1:4 every week with Accutase (Millipore). For neural differentiations, NPCs were dissociated with Accutase and plated at low density in neural differentiation media (DMEM/F12-Glutamax, 1×N2, 1×B27-RA, 20 ng/ml BDNF (Peprotech), 20 ng/ml GDNF (Peprotech), 1 mm dibutyrl-cyclicAMP (Sigma), 200 nM ascorbic acid (Sigma) onto PORN/Laminin-coated plates. Assays for neuronal connectivity, neurite outgrowth, synaptic protein expression, synaptic density, electrophysiology, spontaneous calcium transient imaging and gene expression were used to compare control and SCZD hiPSC neurons. Additional methods are found in S.I.

Description of Schizophrenic Patients

All patient samples were obtained from the Coriell collection. Patients were selected based on the high likelihood of a genetic component to disease. Patient 1 (GM02038, male, 22 years of age, Caucasian) was diagnosed with SCZD at six years of age and committed suicide at 22 years of age. Patient 2 (GM01792, male, 26 years of age, Jewish Caucasian) displayed episodes of agitation, delusions of persecution, and fear of assassination. His sister, patient 3 (GM01835, female, 27 years of age, Jewish Caucasian) had a history of schizoaffective disorder and drug abuse. Patient 4 (GM02497, male, 23 years of age, Jewish Caucasian) was diagnosed with SCZD at age 15 and showed symptoms including paralogical thinking, affective shielding, splitting of affect from content, and suspiciousness. His sister, patient 5 (GM02503, female, 27 years of age, Jewish Caucasian) was diagnosed with anorexia nervosa in adolescence and with schizoid personality disorder (SPD) as an adult. SPD has an increased prevalence in families with SCZD but is a milder diagnosis characterized not by psychosis but rather by a lack of interest in social relationships and emotional coldness (Association, A. P. Diagnostic and statistical manual of mental disorders: DSM-IV. 3rd ed., rev. edn, Vol. 4th ed. (American Psychiatric Press, 1994). Though Applicants show data from SPD patient 5 as an interesting point of comparison, we do not consider patient 5 to belong to either the “control” or “SCZD” groups.

Preliminary experiments were controlled using BJ fibroblasts from ATCC (CRL-2522). These fibroblasts were expanded from foreskin tissue of a newborn male. They are readily reprogrammed, low passage, karyotypically normal and extremely well-characterized primary fibroblast line cells. Age and ancestry matched controls were obtained from three Coriell collections: apparently healthy individuals with normal psychiatric evaluations, apparently healthy non-fetal tissue and gerontology research center cell cultures. hiPSCs were generated from GM02937 (male, 22 years of age), and GM03440 (male, 20 years of age), GM03651 (female, 25 years of age), GM04506 (female, 22 years of age), AG09319 (female, 24 years of age) and AG09429 (female, 25 years of age).

Generation of Lentivirus

Lentivirus was packaged in 293T HEK cells grown in 293T media (IMEM (Invitrogen), 10% FBS (Gemini), 1× Glutamax (Invitrogen)). 293T cells were transfected with Polyethylenimine (PEI) (Polysciences). Per 15-cm plate, the following solution was prepared, incubated for 5 minutes at room temperature and added drop-wise to plates: 12.2 μg lentiviral DNA, 8.1 μg MDL-gagpol, 3.1 μg Rev-RSV, 4.1 μg CMV-VSVG, 500 μl of IMDM and 110 μl PEI (1 μg/μl) and vortexed lightly. Medium was changed after three hours and the virus was harvested at 48 and 72 hours post transfection.

hiPSC Derivation

HFs were cultured on plates treated with 0.1% gelatin (in milliβQ water) for a minimum of 30 minutes and grown in HF media (DMEM (Invitrogen), 10% FBS (Gemini), 1× Glutamax (Invitrogen), 5 ng/ml FGF2 (Invitrogen)).

HFs were infected daily for five days with tetracycline-inducible lentiviruses expressing OCT4, SOX2, KLF4, cMYC and LIN28, driven by a sixth lentivirus expressing the reverse tetracycline transactivator (rtTA)⁷. Cells from a single well of a six-well dish were split onto a 10-cm plate containing 1 million mouse embryonic fibroblasts (mEFs). Cells were switched to HUES media (KO-DMEM (Invitrogen), 10% KO-Serum Replacement (Invitrogen), 10% Plasminate (Talecris), 1× Glutamax (Invitrogen), 1×NEAA (Invitrogen), 1×2βmercaptoethanol (Sigma) and 20 ng/ml FGF2 (Invitrogen)). 1 μg/ml Doxycycline (Sigma) was added to HUES media at for the first 21-28 days of reprogramming.

hiPSC colonies were manually picked and clonally plated onto 24-well mEF plates. hiPSC lines were either maintained on mEFs in HUES media or on Matrigel (BD) in TeSR media (Stemcell Technologies). At early passages, hiPSCs were split through manual passaging. At higher passages, hiPSC could be enzymatically passaged with Collagenase (1 mg/ml in DMEM) (Sigma). Cells were frozen in freezing media (DMEM, 10% FBS, 10% DMSO).

Karyotyping analysis was performed by Cell Line Genetics (Wisconsin, MD) or by Dr. Marie Dell'Aquila (UCSD).

Teratoma analysis was performed by injecting hiPSCs into the kidney capsules of isoflorane-anesthetized NOD-SCID mice. Teratomas were harvested eight weeks post-injection, paraffin-embedded and H&E stained.

hiPSC Differentiation to NPCs and Neurons

hiPSCs grown in HUES media on mEFs were incubated with Collagenase (1 mg/ml in DMEM) at 37° C. for one to two hours until colonies lifted from the plate and were transferred to a nonadherent plate (Corning). Embryoid Bodies (EBs) were grown in suspension in N2 media (DMEM/F12-Glutamax (Invitrogen), 1×N2 (Invitrogen)). After seven days, EBs were plated in N2 media with 1 μg/ml Laminin (Invitrogen) onto polyornithine (PORN)/Laminin-coated plates. Visible rosettes formed within one week and were manually dissected onto PORN/Laminin-coated plates. Rosettes were cultured in NPC media (DMEM/F12, 1×N2, 1×B27-RA (Invitrogen), 1 μg/ml Laminin and 20 ng/ml FGF2) and dissociated in TrypLE (Invitrogen) for three minutes at 37° C. NPCs are maintained at high density, grown on PORN/Laminin-coated plates in NPC media and split approximately 1:4 every week with Accutase (Millipore).

For neural differentiations, NPCs were dissociated with Accutase and plated in neural differentiation media (DMEM/F12, 1×N2, 1×B27-RA, 20 ng/ml BDNF (Peprotech), 20 ng/ml GDNF (Peprotech), 1 mm dibutyrl-cyclicAMP (Sigma), 200 nm ascorbic acid (Sigma) onto PORN/Laminin-coated plates. Density is critical and the following guidelines were used: two-well permanox slide, 80-100,000 cells/well; 24-well, 40-60,000 cells/well; six-well, 200,000 cells/well. hiPSC derived-neurons were differentiated for 1-3 months. Notably, synapse maturation occurs most robustly in vitro when hiPSC neurons are cocultured with wildtype human cerebellar astrocytes (Sciencell). 0.5% FBS was supplemented into neural differentiation media for all astrocyte coculture experiments.

It is difficult to maintain healthy neurons for three months of differentiation and some cultures invariably fail or become contaminated. When even one SCZD patient neural culture failed, the experiments were abandoned as all assays were conducted on neurons cultured in parallel. If, however, only a control neural culture failed, and at least three control samples remained, analysis was completed. For this reason, though patients are consistently numbered throughout the manuscript, controls are not, and are instead listed in numerical order (BJ, GM02937, GM03651, GM04506, AG09319, AG09429).

Antipsychotic drugs were added for the final three weeks of a three-month differentiation on astrocytes and for the final two weeks of a six-week differentiation on PORN/laminin alone. Drugs were resuspended in DMSO at the following concentrations: Clozapine (5 μM), Loxapine (10 μM), Olanzapine (1 μM), Risperidone (10 μM) and Thioridazine (5 μM).

Immunohistochemistry

Cells were fixed in 4% paraformaldehyde in PBS at 4° C. for 10 minutes. hiPSCs and NPCs were permeabilized at room temperature for 15 minutes in 1.0% Triton in PBS. All cells were blocked in 5% donkey serum with 0.1% Triton at room temperature for 30 minutes. The following primary antibodies and dilutions were used: mouse anti-Oct4 (Santa Cruz), 1:200; goat anti-Sox2 (Santa Cruz), 1:200; goat anti-Nanog (R&D), 1:200; mouse anti-Tra1-60 (Chemicon), 1:100; mouse anti-human Nestin (Chemicon), 1:200; rabbit anti-βIII-tubulin (Covance), 1:200; mouse anti-BIII-tubulin (Covance), 1:200; rabbit anti-cow-GFAP (Dako) 1:200; mouse anti-MAP2ab (Sigma), 1:200; rabbit anti-synapsin (Synaptic Systems), 1:500; mouse anti-PSD95 (UCDavis/NIH Neuromab), 1:500; rabbit anti-PSD95 (Invitrogen), 1:200 rabbit-anti-vGlut1 (Synaptic; Systems), 1:500; rabbit anti-Gephyrin, (Synaptic Systems), 1:500; mouse anti-vGat (Synaptic Systems), 1:500; rabbit anti-vGat (Synaptic Systems), 1:500; rabbit anti-GluR1 (Oncogene), 1:100; rabbit anti-GABA (Sigma), 1:200; rabbit anti-GAD67 (Sigma), 1:200.

Secondary antibodies were Alexa donkey 488, 555 and 647 anti-rabbit (Invitrogen), Alexa donkey 488 and 555 anti-mouse (Invitrogen), and Alexa donkey 488, 555, 568 and 594 anti-goat (Invitrogen); all were used at 1:300. To visualize nuclei, slides were stained with 0.5 μg/ml DAPI (4′,6-diamidino-2-phenylindole) and then mounted with Vectashield. Images were acquired using a Bio-Rad confocal microscope.

FACS

For sorting of dissociated hiPSC-derived neurons, cultures were dissociated in trypsin for 5 minutes, washed in DMEM, centrifuged at 500×g and resuspended in PBS. Cells were fixed in 4% paraformaldehyde in PBS at 4° C. for 10 minutes. Cells were washed in PBS and aliquoted into 96-well conical plates. Cells were blocked in 5% donkey serum with 0.1% saponin at room temperature for 30 minutes. The following primary antibodies and dilutions were used for one hour at room temperature: rabbit anti-βIII-tubulin (Sigma), 1:200; mouse anti-MAP2a+b (Sigma), 1:100. Cells were washed and then incubated with secondary antibodies at 1:200 for 30 minutes at room temperature: Alexa donkey 647 anti-rabbit (Invitrogen), and Alexa donkey 488 anti-mouse (Invitrogen). Cells were washed three times in PBS and stained with 0.5 μm/ml DAPI (4′,6-diamidino-2-phenylindole). Cells were resuspended in PBS with 5% donkey serum and 0.1% detergent saponin. The homogeneous solution was filtered through a 250-μM nylon sieve and run in a BD FACS Caliber. Data were analyzed using FloJo.

Rabies Virus Trans-Neuronal Tracing

Rabies virus trans-neuronal tracing was performed on three-month-old hiPSC neurons cocultured with wildtype human astrocytes (Sciencell) on acid-etched glass coverslips and then transduced with LV-SYNP-HTG or LV-SYNP-HT. Cultures were transduced with Rabies-ENVAΔG-RFP after at least a week to allow expression of ENVA and rabies G. Either 5, 7 or 10 days later, hiPSC neurons were either dissociated with accutase for FACS analysis of fixed with 4% paraformaldehyde in PBS for fluorescent microscopy.

Neurite Analysis

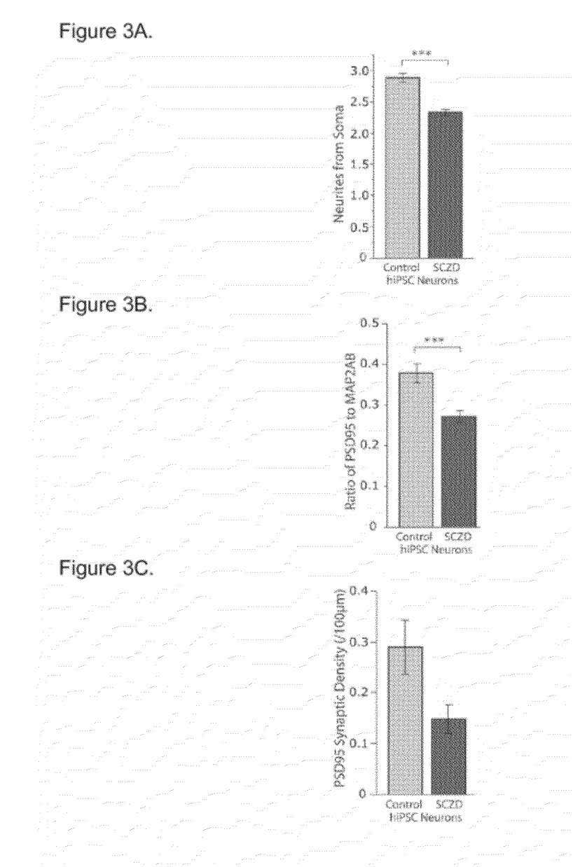

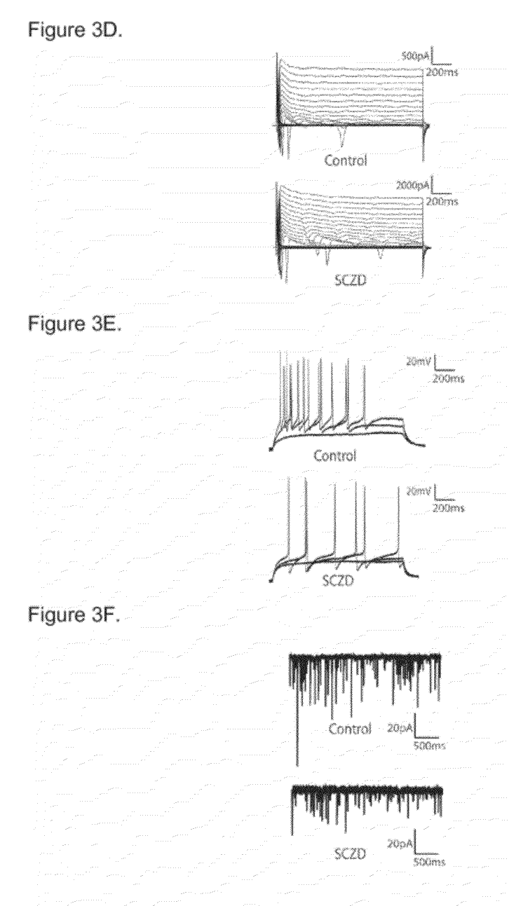

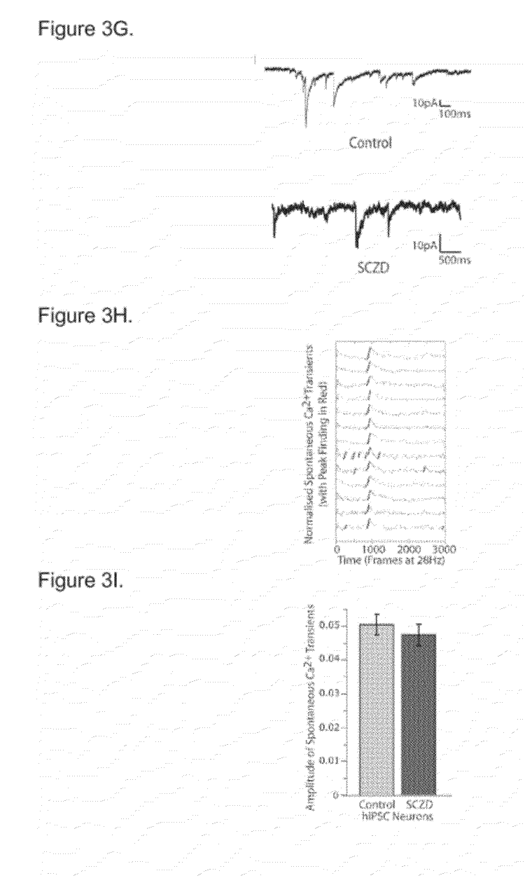

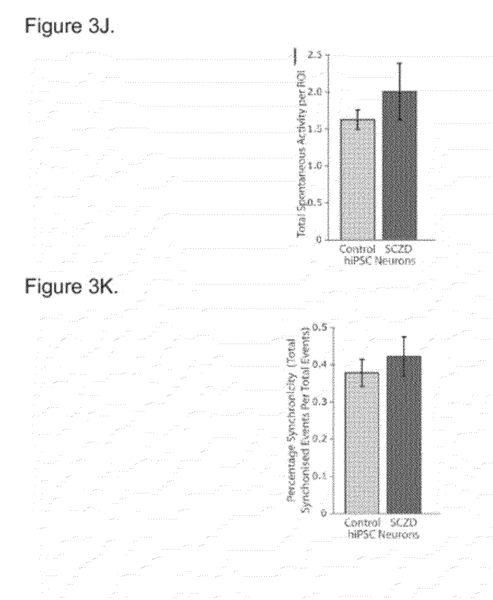

Neurite analysis was performed on three-month-old hiPSC neurons cocultured with wildtype human astrocytes (Sciencell) on acid-etched glass. Low titer transduction of a lentivirus driving expression of GFP from the SYN promoter (LV-SYNP-GFP) occurred at least 7 days prior to assay. LV-SYNP-GFP was used to image and count branching neurites from single neurons (FIG. 3A). The number of neurites extending from the soma of 691 single LV-SYNP-GFP-labeled neurons was determined by a blinded count.

Synaptic Protein Staining Analysis

Synaptic protein staining was performed on three-month-old hiPSC neurons cocultured with wildtype human astrocytes (Sciencell) on acid-etched glass. To calculate ratios of MAP2AB-positive dendrites and synaptic proteins, confocal images were taken at 630× magnification and 4× zoom. Using NIH ImageJ, images were thresholded and the integrated pixel density was determined for each image. Integrated pixel density measurement is the product of area (measured in square pixels) and mean gray value (the sum of the gray values of all the pixels in the selection divided by the number of pixels).

Synapse Density

Manual counts of synaptic density were done in three steps using NIH ImageJ. First, the colocalization plugin was used to identify colocalization of VGLUT1 and PSD95. Second, the particle analysis function was used to restrict size 50-infinity. Third, dendrites were traced using the NeuronJ plugin. The mask generated by particle analysis was overlayed on the trace generated by NeuronJ and synapses were manually counted.

Electrophysiology

Whole-cell perforated patch recordings were performed on SCZD (n=30) and control (n=20) three-month-old hiPSC neurons cocultured with wildtype human astrocytes (Sciencell) on acid-etched coverslips and typically transduced with LV-SYNP-GFP. The recording micropipettes (tip resistance 3-6 MÙ) were tip-filled with internal solution composed of 115 mM K-gluconate, 4 mM NaCl, 1.5 mM MgCl2, 20 mM HEPES, and 0.5 mM EGTA (pH 7.4) and then back-filled with the same internal solution containing 200 μg/ml amphotericinB (Calbiochem). Recordings were made using Axopatch 200B amplifier (Axon Instruments). Signals were sampled and filtered at 10 kHz and 2 kHz, respectively. The whole-cell capacitance was fully compensated, whereas the series resistance was uncompensated but monitored during the experiment by the amplitude of the capacitive current in response to a 5 mV pulse. The bath was constantly perfused with fresh HEPES-buffered saline composed of 115 mM NaCl, 2 mM KCl, 10 mM HEPES, 3 mM CaCl₂, 10 mM glucose and 1.5 mM MgCl₂ (pH 7.4). For voltage-clamp recordings, cells were clamped at −60 to −80 mV; Na+ currents and K+ currents were stimulated by voltage step depolarizations. Command voltage varied from −50 to +20 mV in 10 mV increments. For current-clamp recordings, induced action potentials were stimulated with current steps from −0.2 to +0.5 nA. All recordings were performed at room temperature.

Spontaneous Calcium Transients

Culture media was removed and hiPSC cultures were incubated with 0.4 μM Fluo-4AM (Molecular Probes) and 0.02% Pluronic F-127 detergent in Krebs HEPES Buffer (KHB) (10 mM HEPES, 4.2 mM NaHCO₃, 10 mM dextrose, 1.18 mM MgSO₄.2H₂O, 1.18 mM KH₂PO₄, 4.69 mM KCl, 118 mM NaCl, 1.29 mM CaCl₂; pH 7.3) for one hour at room temperature. Cells were washed with KHB buffer, incubated for two minutes with Hoechst dye diluted 1:1000 in KHB, and allowed to incubate for an additional 15 minutes in KHB to equilibrate intracellular dye concentration. Time lapse image sequences (100× magnification) were acquired at 28 Hz using a Hamamatsu ORCA-ER digital camera with a 488 nm (FITC) filter on an Olympus IZ81 inverted fluorescence confocal microscope. Images were acquired with MetaMorph.

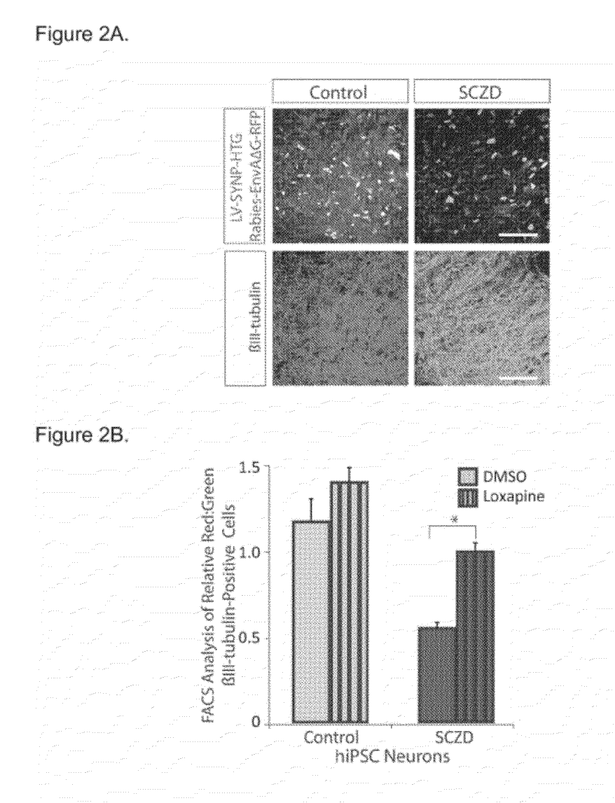

In total, eight independent neural differentiations were tested per patient, 210 movies of spontaneous calcium transients (110 control and 100 schizophrenic) were generated and 2,676 ROIs (1,158 control and 1,518 schizophrenic ROIs) were analyzed. Up to four 90-second videos of Fluo-4AM fluorescence were recorded per neural differentiation per patient with a spinning disc confocal microscope at 28 frames per second (Supplementary FIG. 2A). Using ImageJ software, regions of interest (ROIs) can be manually selected and the mean pixel intensity of each ROI can be followed over time, generating time trace data for each ROI. The data were analyzed in Matlab where background subtraction was performed by normalizing traces among traces of the sample, and spike events were identified based on the slope and amplitude of the time trace.

The amplitude of spontaneous calcium transients was calculated by measuring the change in total pixel intensity for each normalized calcium transient trace. The rate was determined by dividing the total number of spontaneous calcium transients for any ROI by the total length of the movie (90 seconds). The synchronicity of spontaneous calcium transients was determined by two independent calculations. First, to determine the percentage synchronicity per calcium transient, the total number of synchronized calcium transients, defined as three or more simultaneous peaks, was divided by the total number of spontaneous calcium transients identified. Second, to calculate the maximum percentage synchronicity, the maximum number of ROIs involved in a single synchronized event was divided by the total number of ROIs identified.

CNV Analysis

Cells were lysed in DNA Lysis solution (100 mM Tris, pH 8.5, 5 mM EDTA, 200 mM NaCl, 0.2% (w/v) sarcosyl, and 100 μg/ml fresh proteinase K) overnight at 50° C. DNA was precipitated by the addition of an equal volume of NaCl-ethanol mixture (150 μl of 5 M NaCl in 10 ml cold 95% ethanol) and then washed three times in 70% ethanol prior to resuspension in water with RNAseA overnight at 4° C.

Genome Scans were performed using NimbleGen HD2 arrays (NimbleGen Systems Inc) according the to the manufacturer's instructions using a standard reference genome SKN1. NimbleGen HD2 dual-color intensity data were normalized in a two-step process: first, a ‘spatial’ normalization of probes was performed to adjust for regional differences in intensities across the surface of the array, and second, the Cy5 and Cy3 intensities were adjusted to a fitting curve by invariant set normalization, preserving the variability in the data. The log 2 ratio for each probe was then estimated using the geometric mean of normalized and raw intensity data (McCarthy, S. E. et al., Nature Genetics 41:1223-1227).

CNV analysis was completed to identify deletions and duplications present within the patients. By using a virtual “genotyping” step whereby individual CNV segment probe ratios were converted into z-scores, a distribution of median Z-scores were generated, outliers of which were considered to be true CNVs. In doing so, Applicants better filtered out common artifacts and false positive CNVs and generated a list of CNVs unbiased by previous genetic studies of schizophrenia.

Patient fibroblasts were used for CNV analysis. Lymphocytes were available for patient 4 and his parents, allowing us to validate the CNVs identified for patient 4 and also determine the parent of origin for each mutation; many were inherited from the unaffected mother (Table 8)

Gene Expression Analysis

Cells were lysed in RNA BEE (Tel-test, Inc). RNA was chloroform extracted, pelleted with isopropanol, washed with 70% ethanol and resuspended in water. RNA was treated with RQ1 RNAse-free DNAse (Promega) for 30 minutes at 37° C. and then the reaction was inactivated by incubation with EGTA Stop buffer at 65° C. for 10 minutes.

For gene expression arrays, three independent neural differentiations for each of the 4 schizophrenic patients, as well as 4 control subjects, were compared using Affymetrix Human 1.0ST arrays as specified by the manufacturer.

Gene expression array analysis was completed using Partek software. Pathway analysis was performed using Metacore GeneGo.

For qPCR, cDNA was synthesized using Superscript III at 50° C. for one to two hours, inactivated for 15 minutes at 70° C. and then treated with RNAaseH for 15 minutes at 37° C., inactivated with EDTA and heated to 70° C. for 15 minutes. qPCR was performed using SybrGreen. Primers used are listed in Table 8.

Statistical Analysis

Statistical analysis was completed using JMP software. Data was transformed into a normal distribution using a box-cox transformation. The Shapiro-Wilk W test was performed to ensure a normal distribution. Means were compared within diagnosis by One way analysis using both Student's T test and Tukey Kramer HSD. Finally, a nested analysis of values for individual patients and controls was performed using standard least squares analysis comparing means for all pairs using both Student's T test and Tukey Kramer HSD.

### B. Results

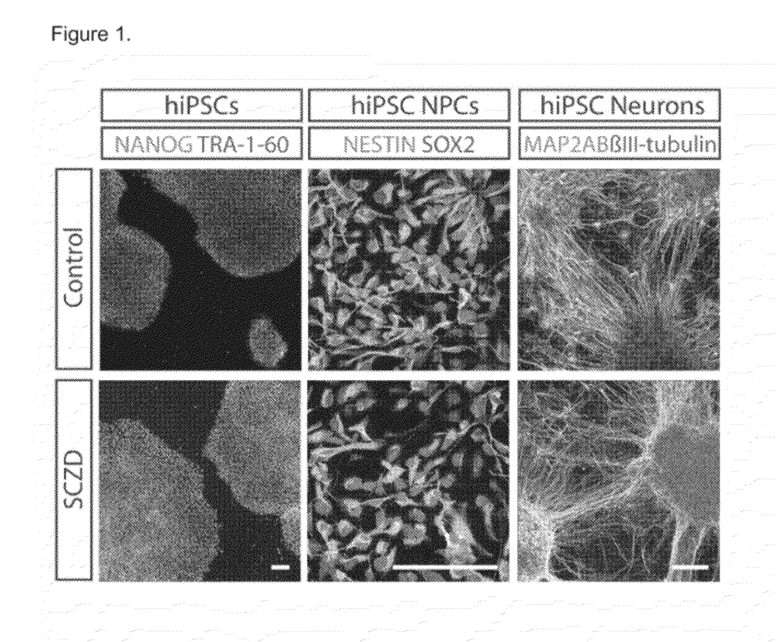

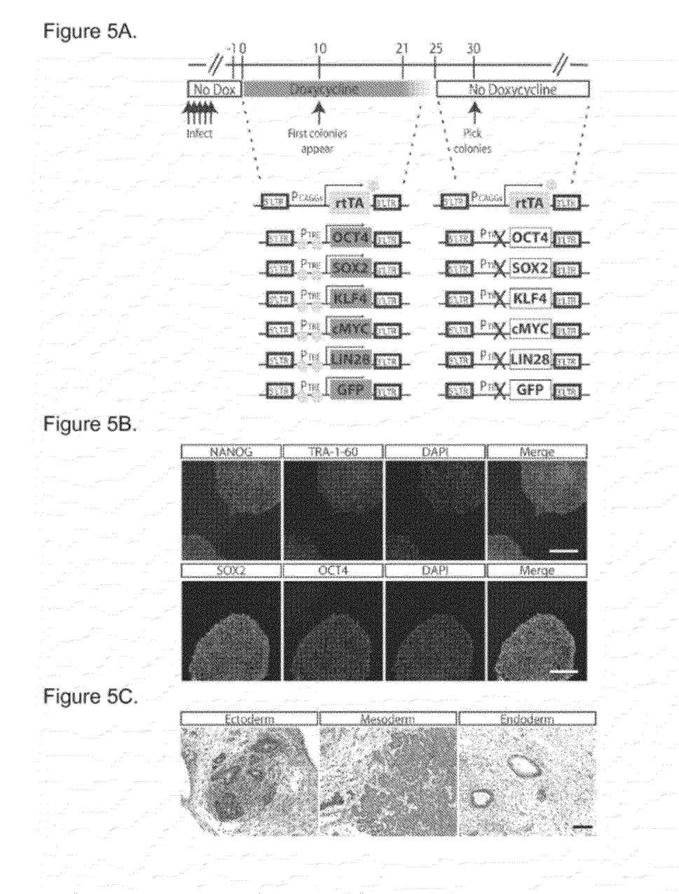

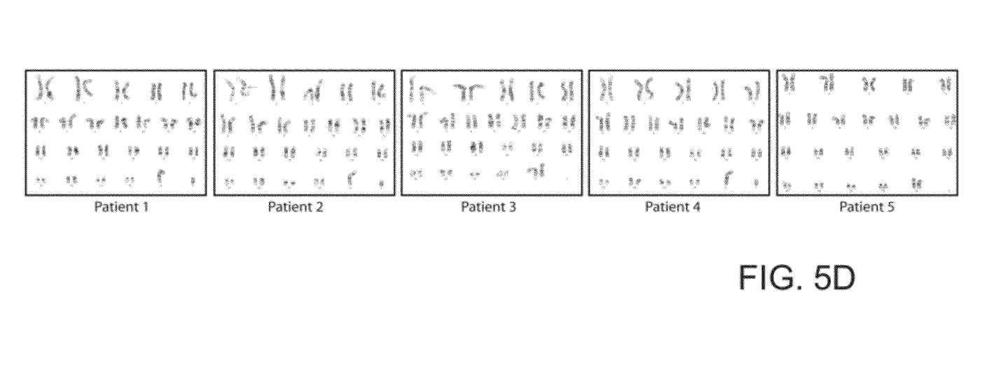

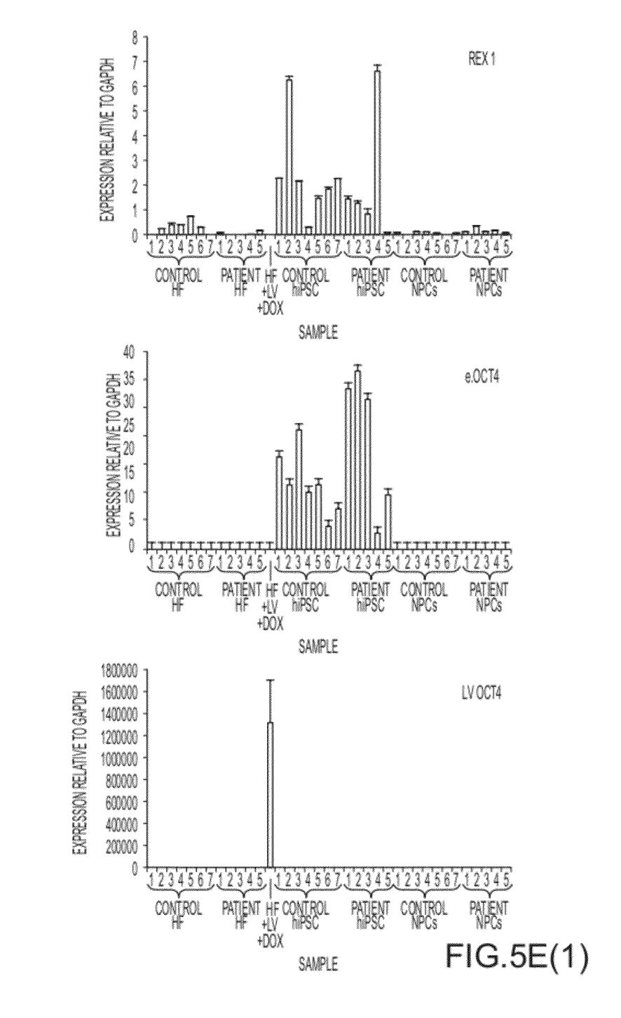

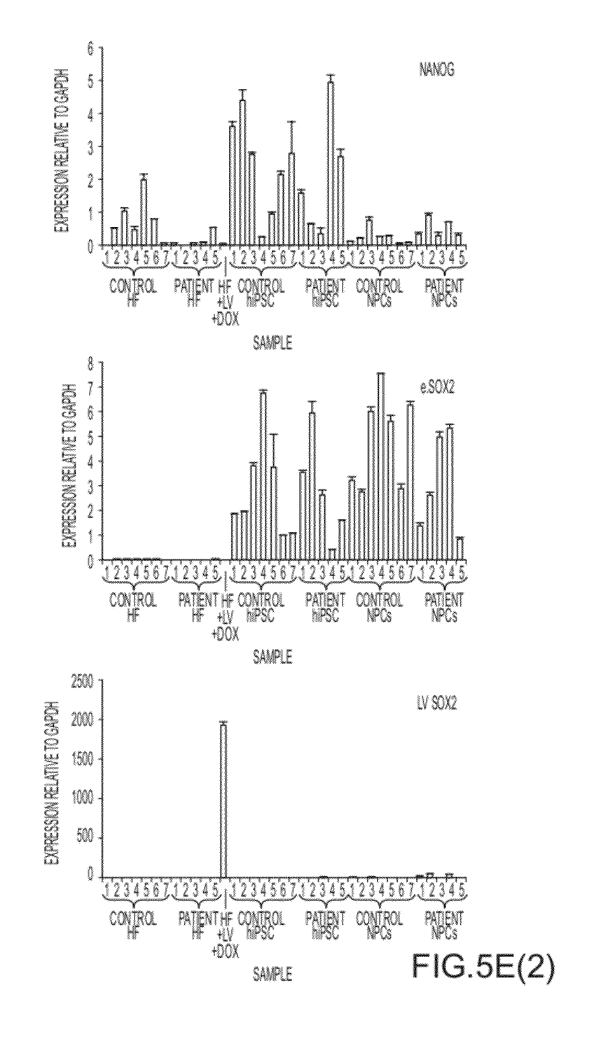

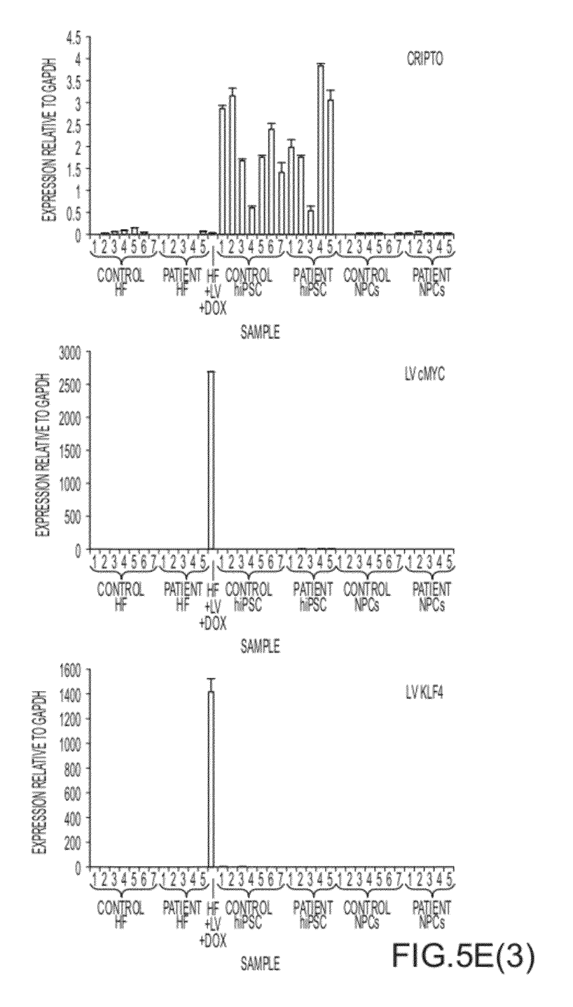

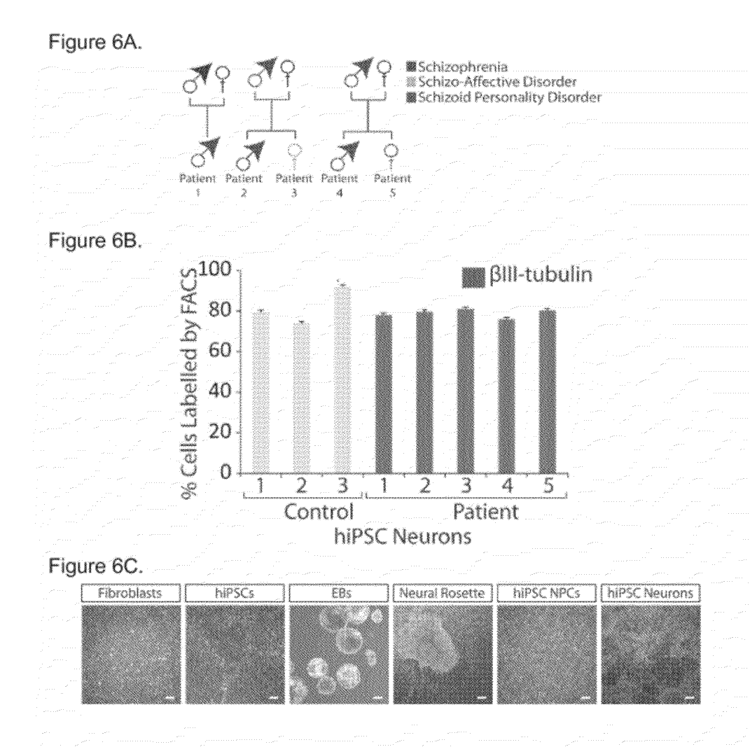

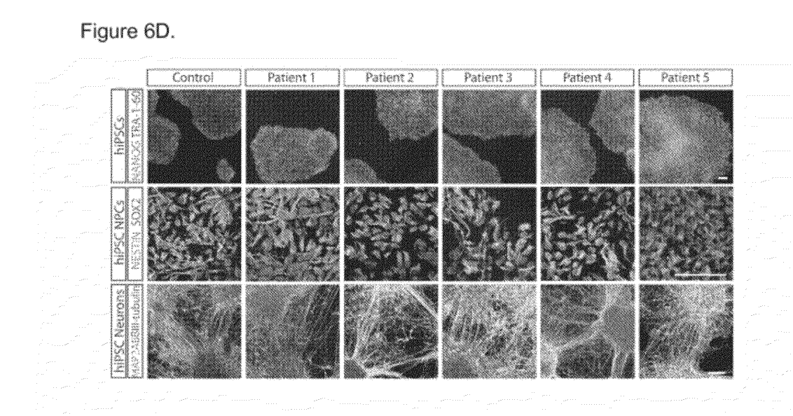

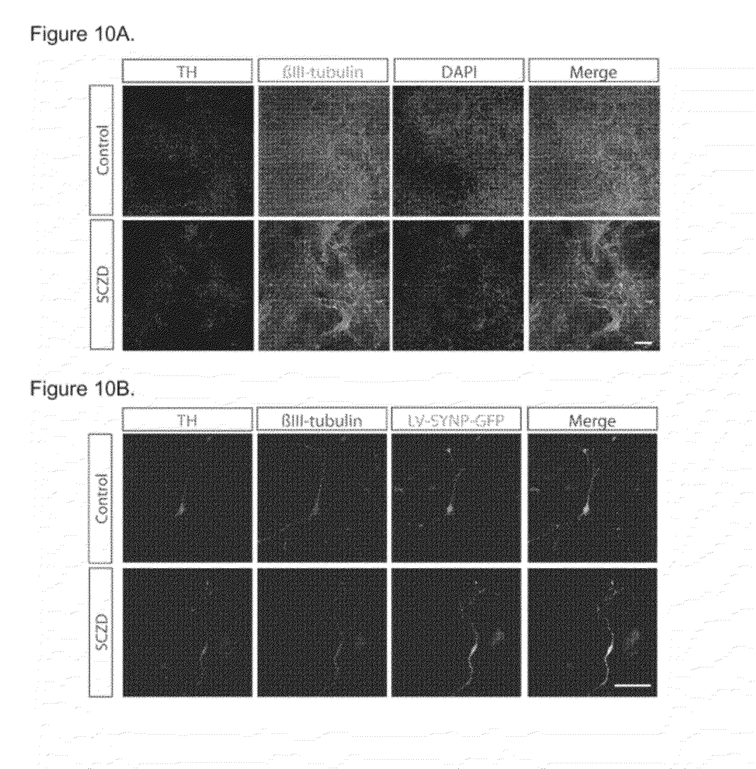

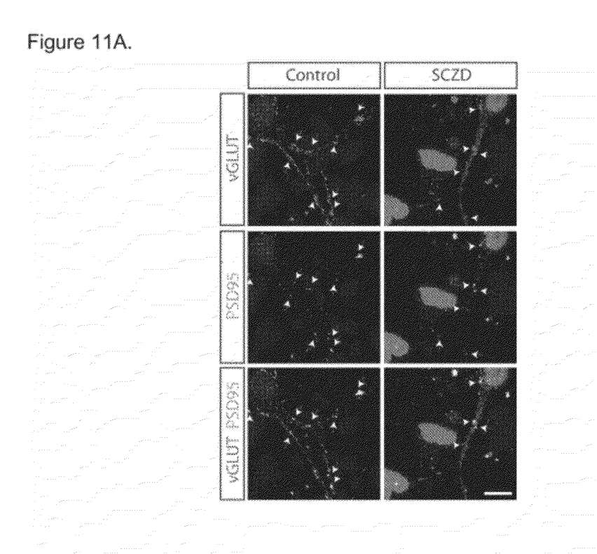

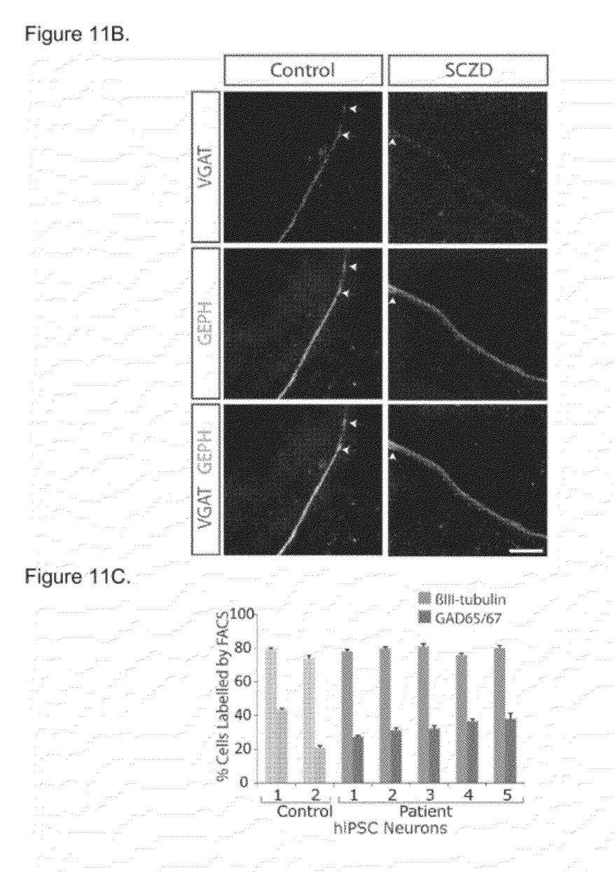

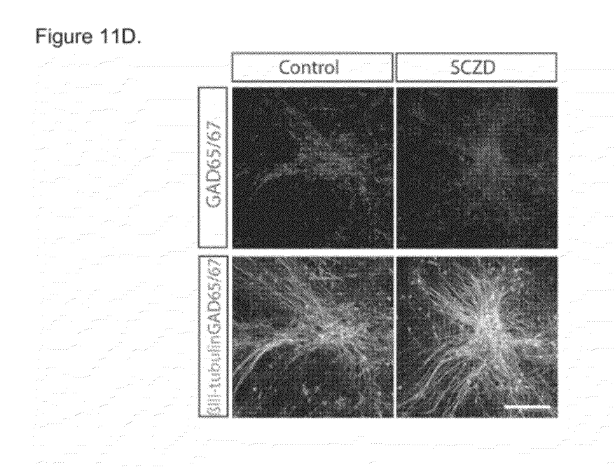

Four SCZD patients were selected: patient 1, diagnosed at six years of age, had childhood-onset SCZD; patients 2, 3 and 4 were from families in which all offspring and one parent were affected with psychiatric disease. Primary human fibroblasts (HFs) were reprogrammed using inducible lentiviruses⁷. Control and SCZD hiPSCs expressed endogenous pluripotency genes, repressed viral genes and were indistinguishable in assays for self-renewal and pluripotency (FIG. 1). SCZD hiPSCs had no apparent defects in generating neural progenitor cells (NPCs) or neurons (FIG. 1; FIG. 5). Most hiPSC neurons were presumably glutamatergic and expressed VGLUT1 (FIG. 11A). Approximately 30% of neurons were GAD67-positive (GABAergic) (FIG. 11C,D) whereas less than 10% of neurons were tyrosine hydroxylase (TH)-positive (dopaminergic) (FIG. 10).

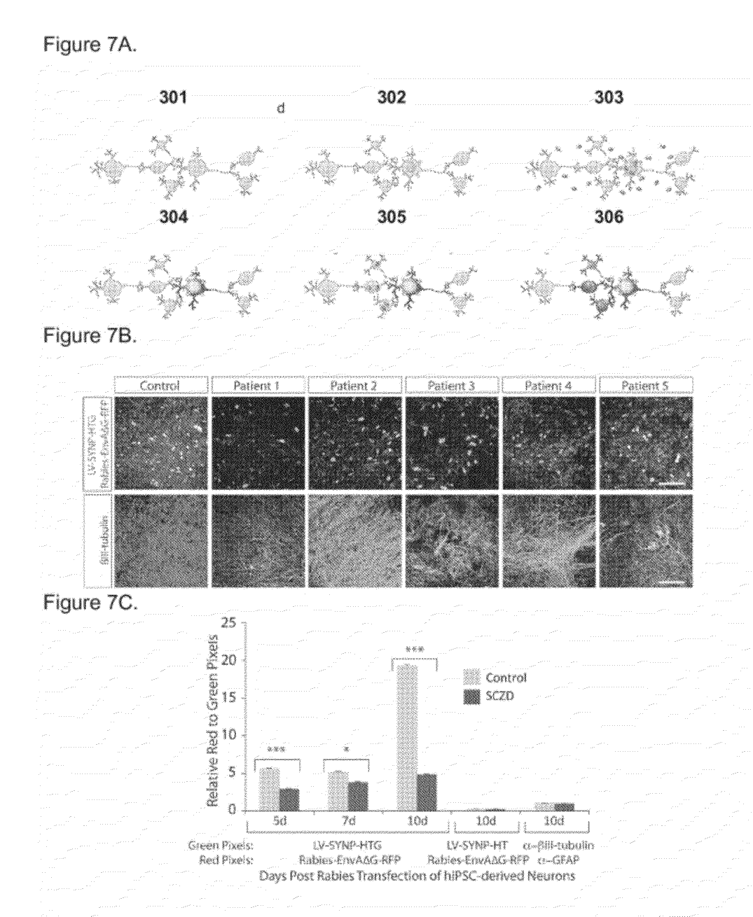

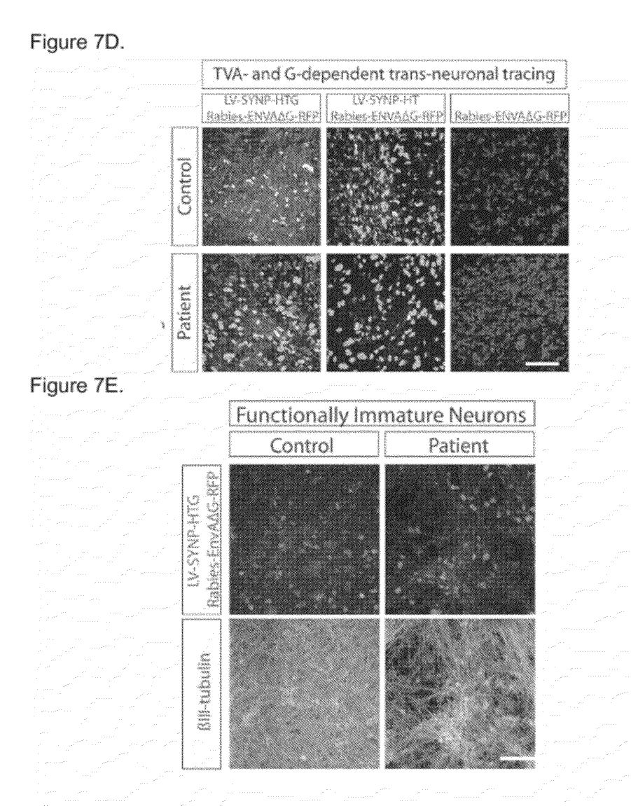

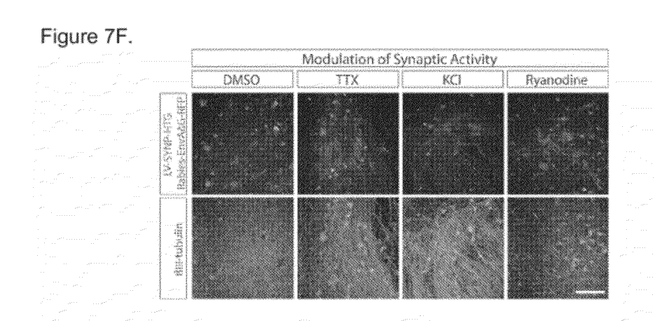

Neuronal connectivity was assayed using trans-neuronal spread of rabies; in vivo, rabies transmission occurs via synaptic contacts and is strongly correlated with synaptic input strength⁸. Primary infection was restricted by replacing the rabies coat protein with envelope A (ENVA), which infects only via the avian tumor virus A (TVA) receptor; viral spread was limited to monosynaptically connected neurons by deleting the rabies glycoprotein gene (AG)⁹. Neurons were first transduced with a lentivirus expressing Histone 2B (H2B)-green fluorescent protein (GFP) fusion protein, TVA and G from the synapsin (SYN) promoter (LV-SYNP-HTG). One week later, neurons were transduced with modified rabies (Rabies-ENVAΔG-RFP). Primary infected cells were positive for both H2BGFP and RFP; neurons monosynaptically connected to primary cells were GFP-negative but RFP-positive (FIG. 7A). Transduction with Rabies-ENVAΔG-RFP alone resulted in no RFP-positive cells, whereas transduction with Rabies-ENVAΔG-RFP following lentiviral transduction without rabies glycoprotein (SYNP-HT) led to only single GFP⁺ RFP⁺ cells, indicating that in vitro rabies infection and spread are dependent on TVA expression and G trans-complementation, respectively (FIG. 7C,D).

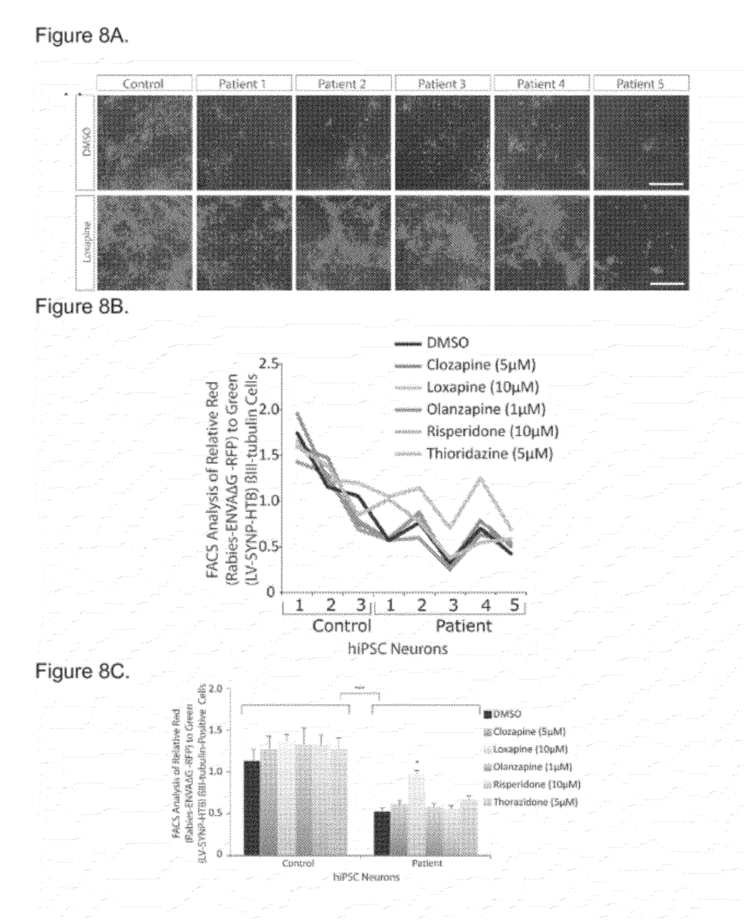

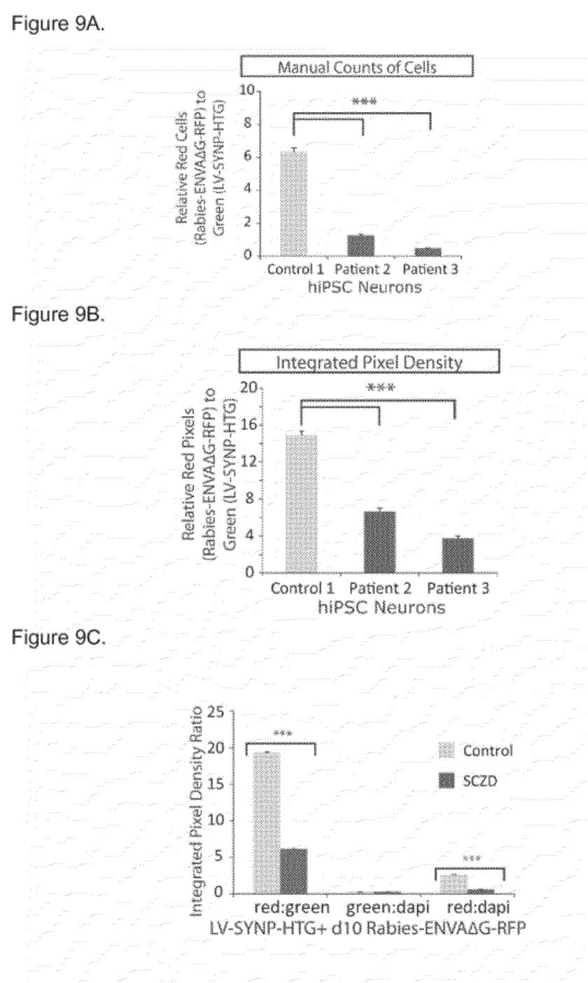

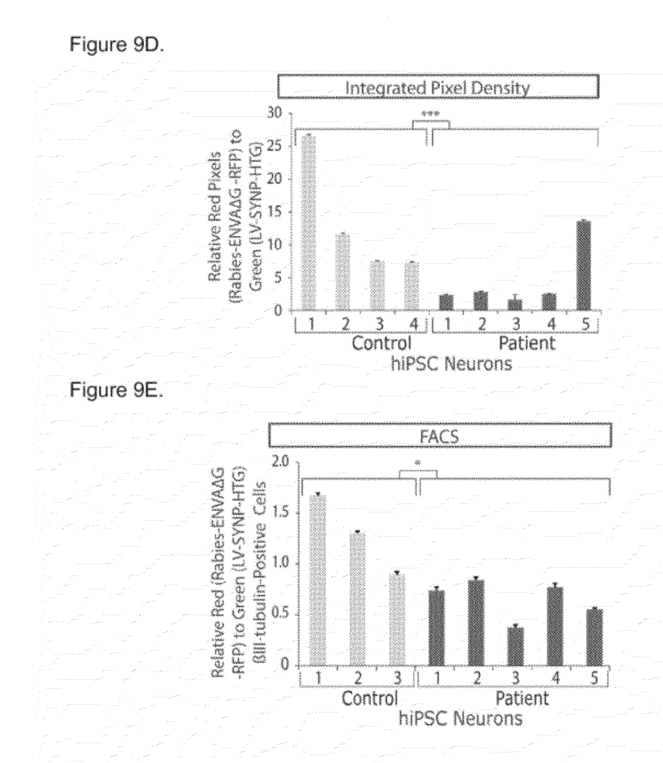

There was decreased neuronal connectivity in SCZD hiPSC neurons (FIG. 2; FIG. 8B,C; FIG. 8,5). FACS analysis confirmed differences in neuronal connectivity and demonstrated that comparable numbers of βIII-tubulin-positive neurons were labeled with LV-SYNP-HTG. Though the mechanism of rabies trans-neuronal tracing is not fully understood, the presynaptic protein NCAM has been implicated¹⁰; NCAM expression is decreased in SCZD hiPSC neurons (Table 5). Rabies trans-neuronal tracing occurs in functionally immature hiPSC neurons (FIG. 7E) and in the presence of the voltage-gated sodium channel blocker tetrodotoxin (TTX) (1 μM), depolarizing KCl (50 mM) or the calcium channel blocker ryanodine (10 μM) (FIG. 7F). Decreased trans-neuronal tracing is evidence of decreased neuronal connectivity, but not necessarily decreased synaptic function, in SCZD hiPSC neurons.

Applicants tested the ability of five antipsychotic drugs to improve neuronal connectivity in vitro. Clozapine, Loxapine, Olanzapine, Risperidone and Thioridazine were administered for the final three weeks of neuronal differentiation. Only Loxapine significantly increased neuronal connectivity in hiPSC neurons from all patients (FIG. 2B; FIG. 8). Optimization of the concentration and timing of drug administration may improve the effects of the other antipsychotic medications.

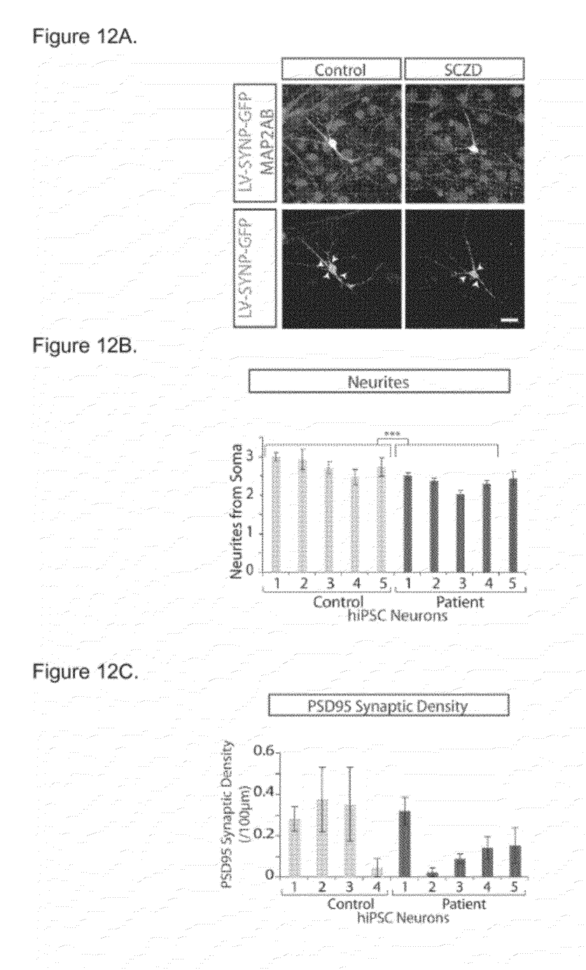

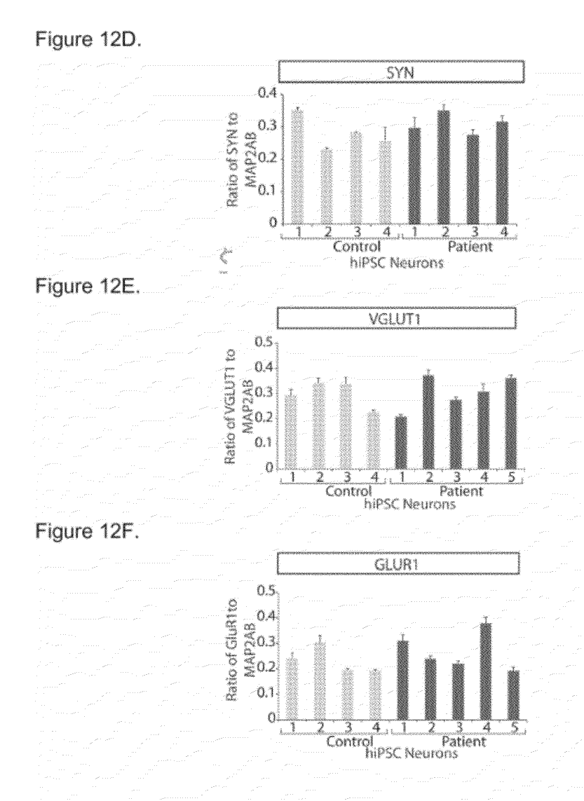

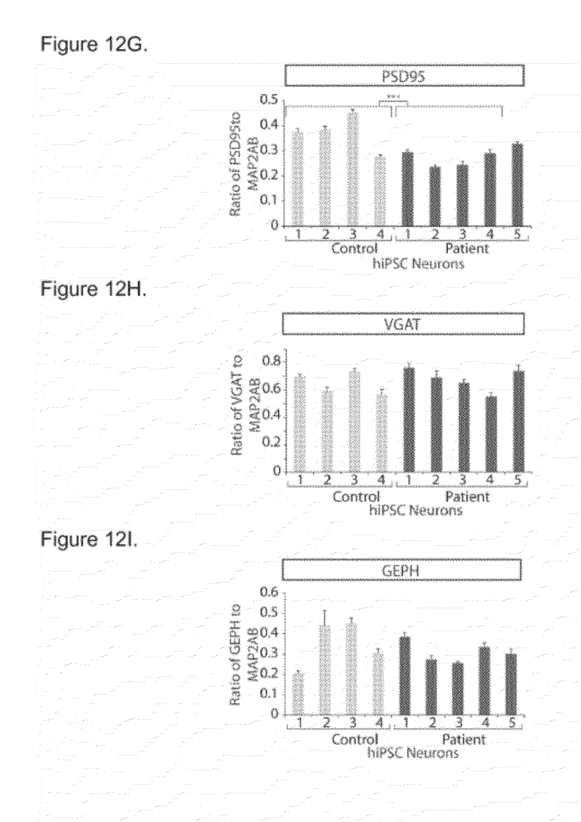

Reduced dendritic arborization has been observed in postmortem SCZD brains¹¹ and in animal models¹². SCZD hiPSC neurons show a decrease in the number of neurites (FIG. 3A; FIG. 12A,B). Synaptic genes are associated with SCZD¹³ (FIG. 12D) and impaired synaptic maturation occurs in a number of mouse models¹². hiPSC neurons express dense puncta of synaptic markers that co-stain for both pre- and post-synaptic markers (FIG. 11A,B). While Applicants observed decreased PSD95 protein expression relative to MAP2AB in SCZD hiPSC neurons (FIG. 3B; FIG. 12H), the levels of SYN, VGLUT1, GLUR1, VGAT and GEPH were unaffected (FIG. 12E-I). Decreased PSD95 synaptic density in SCZD hiPSC neurons failed to reach statistical significance (FIG. 3C; FIG. 12C).

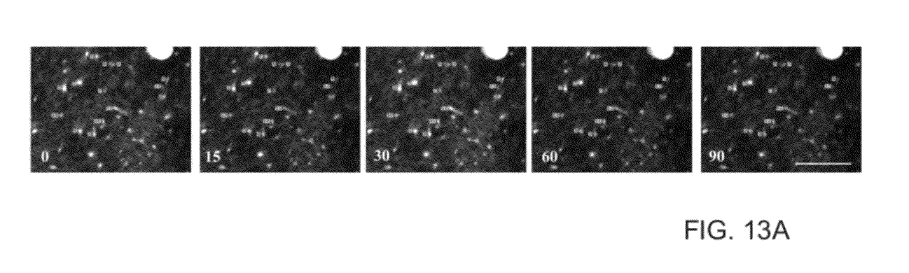

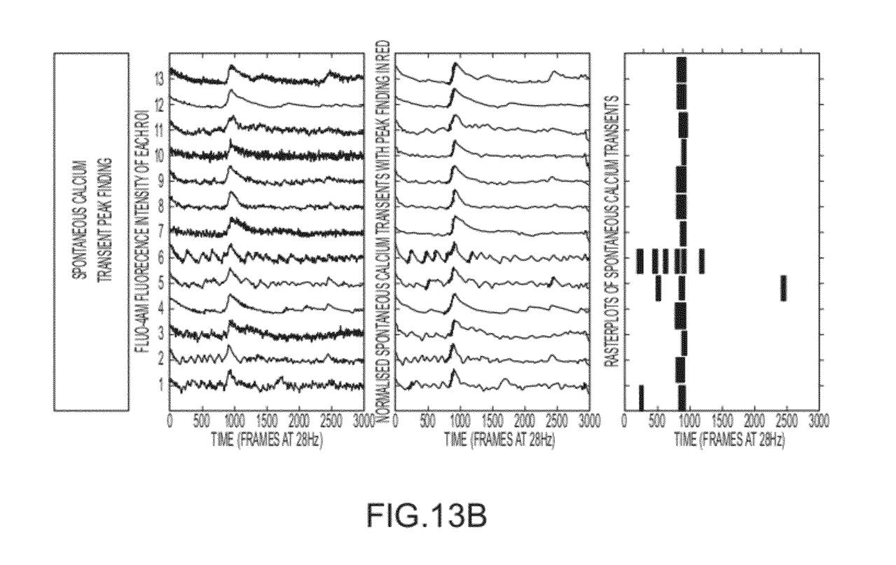

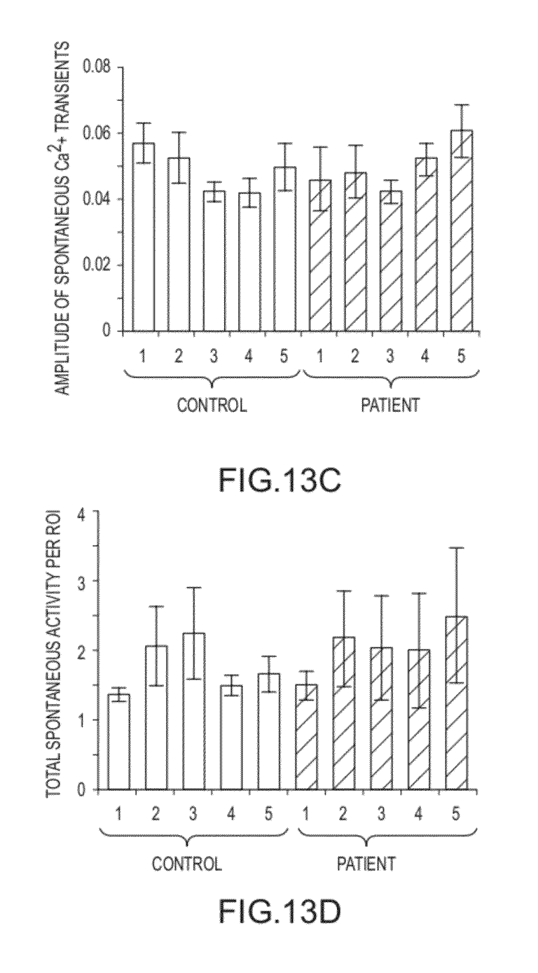

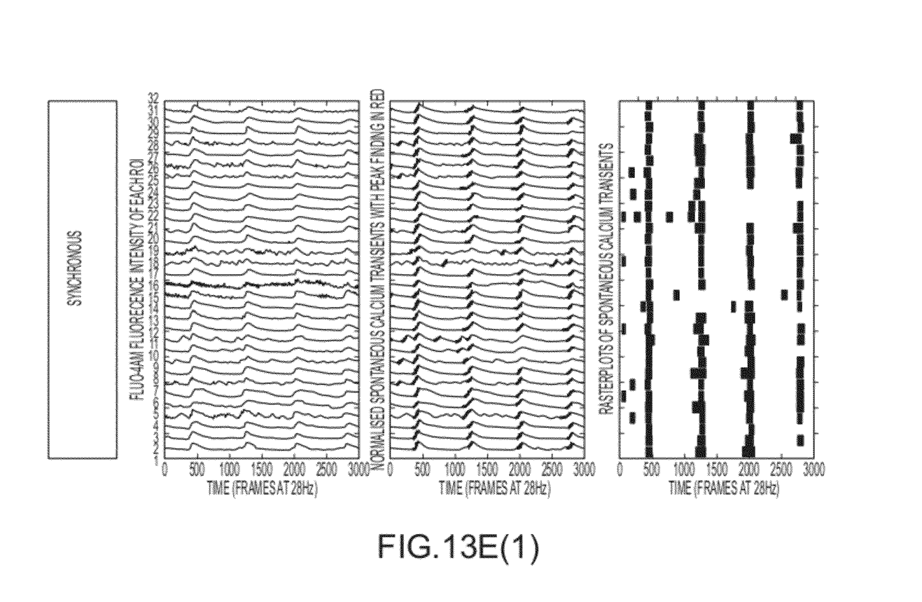

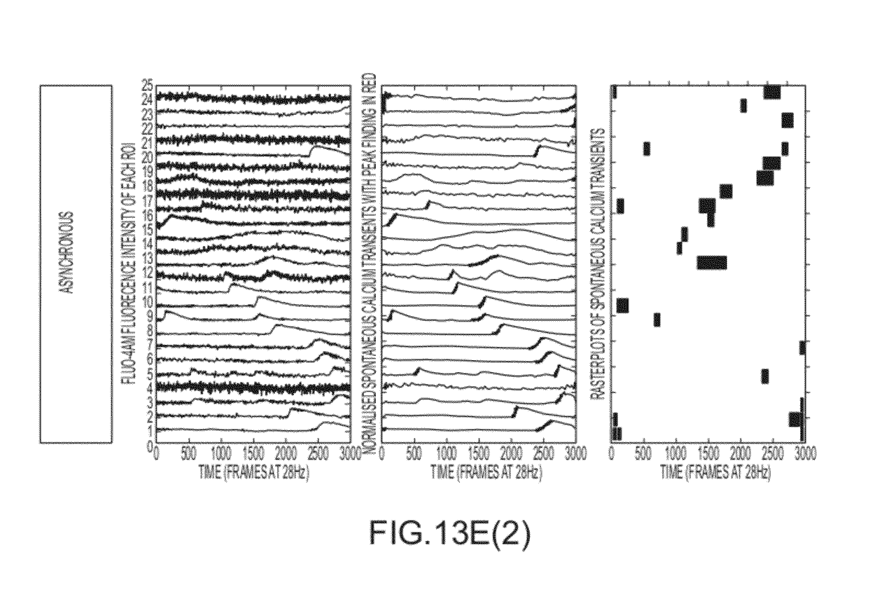

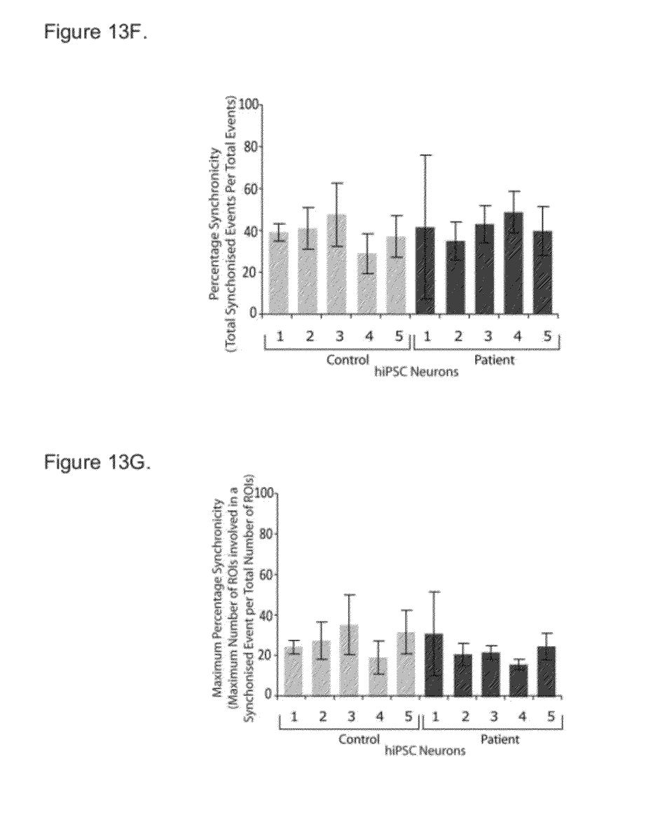

Applicants used electrophysiology and calcium transient imaging to measure spontaneous neuronal activity (FIG. 3D-K; FIG. 13). SCZD hiPSC neurons showed normal transient inward sodium currents and sustained outward potassium currents in response to membrane depolarizations (FIG. 3D), action potentials to somatic current injections, (FIG. 3E), excitatory postsynaptic currents (EPSCs) (FIG. 3F) and inhibitory postsynaptic currents (IPSCs) (FIG. 3G). The amplitude and rate of spontaneous calcium transients were unaffected (FIG. 3H-J; FIG. 13A-D) and there was no difference in synchronicity of spontaneous calcium transients (FIG. 3K; FIG. 13E-G).

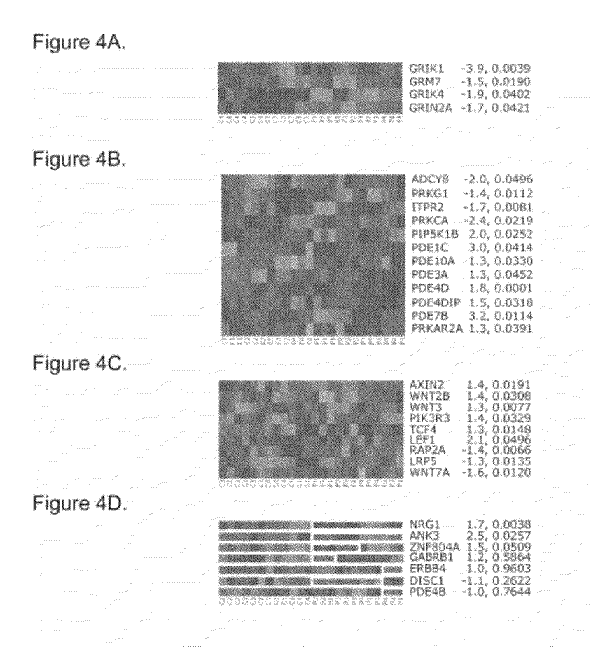

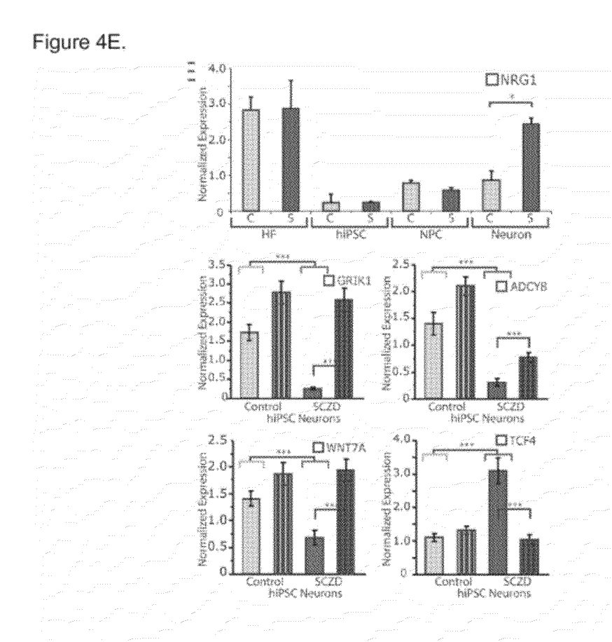

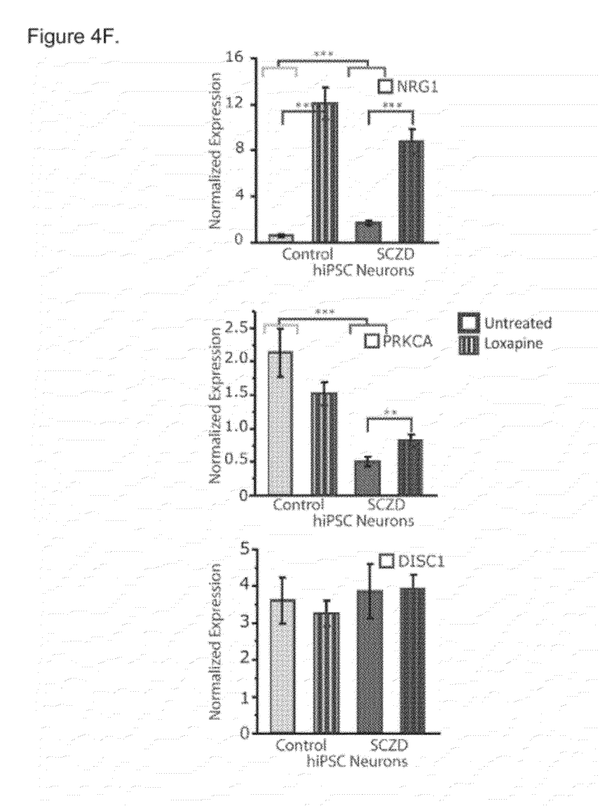

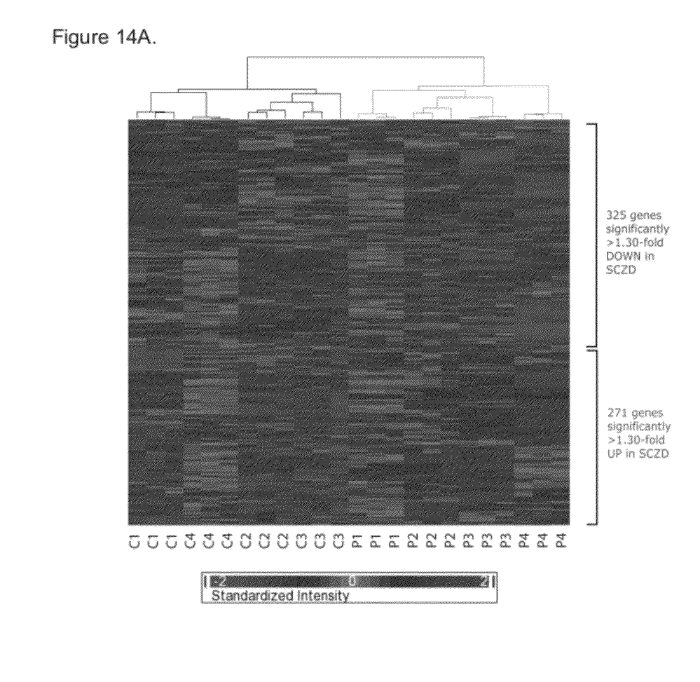

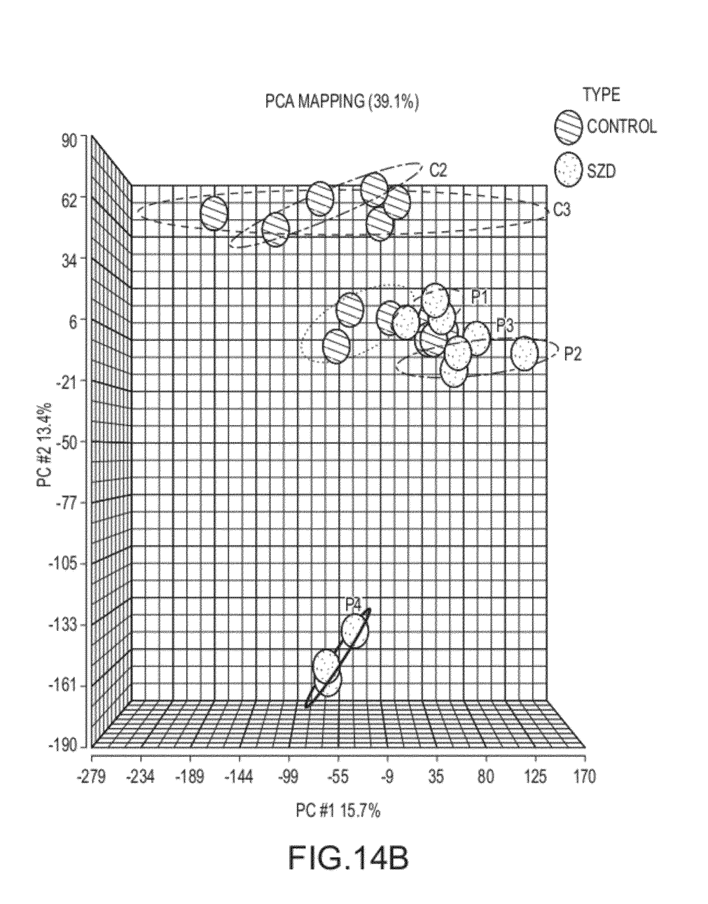

Increased NRG1 expression has been observed in postmortem SCZD brain tissue¹³. NRG1 expression was increased in SCZD hiPSC neurons (FIG. 4D-F) but not SCZD fibroblasts (HF), hiPSCs or NPCs (FIG. 4E), demonstrating the importance of studying gene expression changes in the cell type relevant to disease. In all, 596 unique genes (271 upregulated and 325 downregulated) showed greater than 1.30-fold-expression changes between SCZD and control hiPSC neurons (p<0.05) (FIG. 14A,B; Table 5). Of these genes, 13% (74) have published associations with SCZD and 16% (96) have been linked to SCZD by postmortem gene expression profiles available through the Stanley Medical Research Institute¹⁴ (Table 5); in total 25% (149) of the differentially expressed genes have been previously implicated in SCZD. Gene ontology (GO) analysis identified significant perturbations of glutamate, cAMP and WNT signaling (FIG. 4A-C; Table 6), pathways required for activity-dependent refinement of synaptic connections and long-term potentiation¹⁵⁻¹⁷. Sixteen of 17 candidate genes from these families were validated by qPCR (Table 4; FIG. 4F; FIG. 14C).

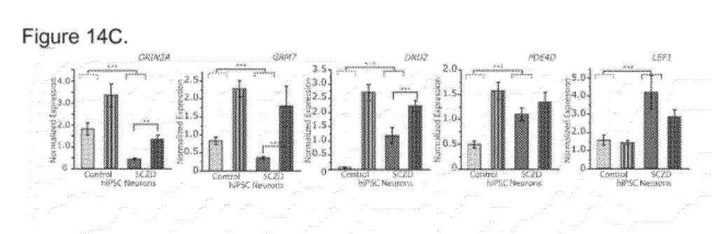

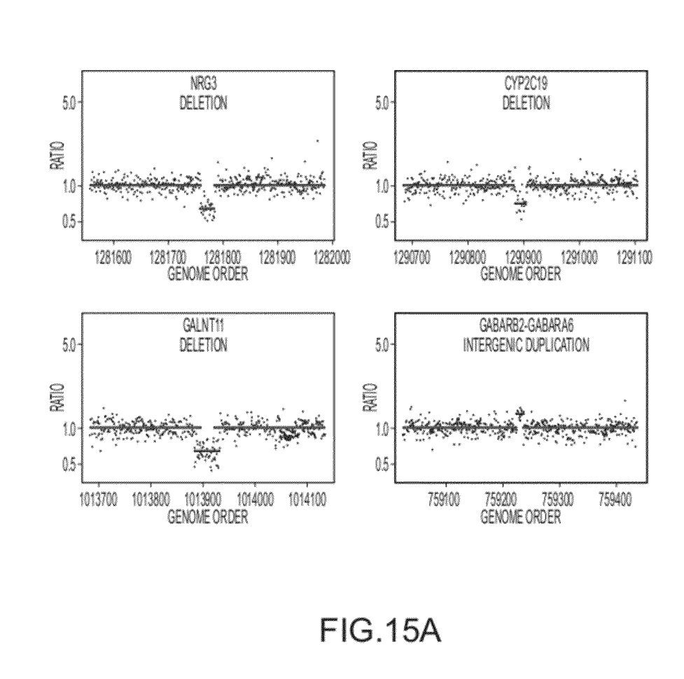

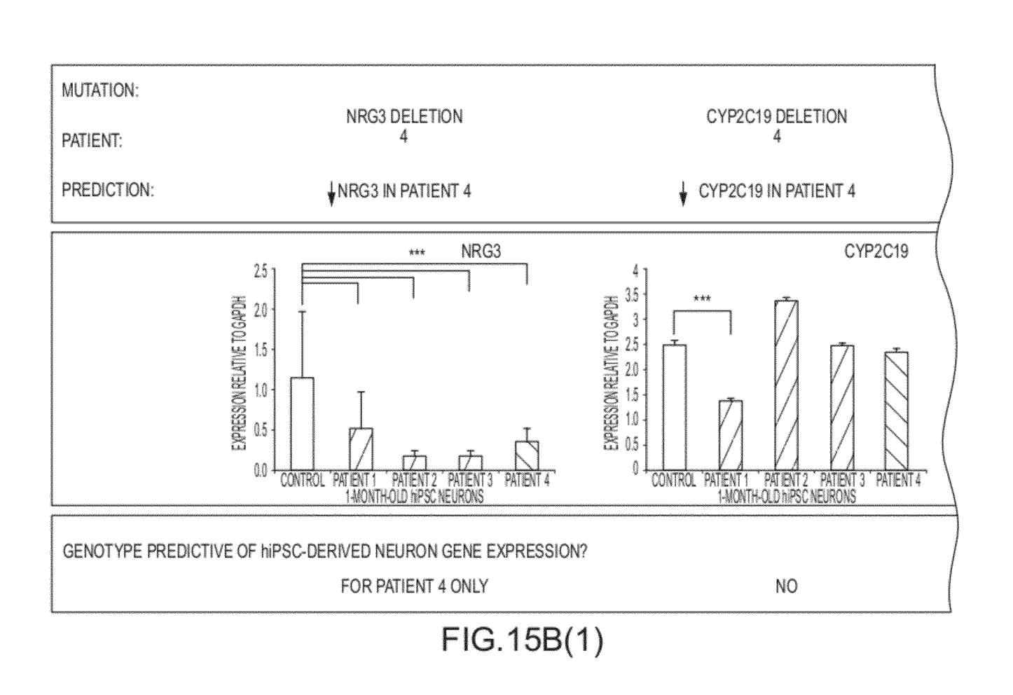

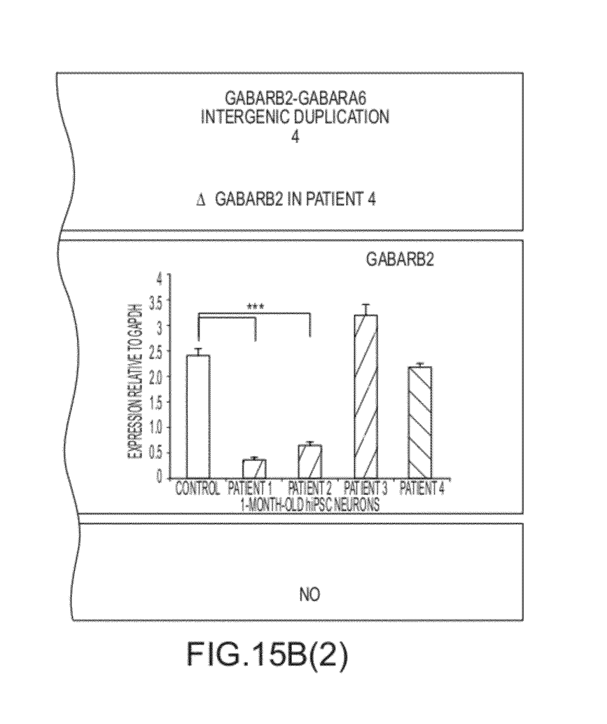

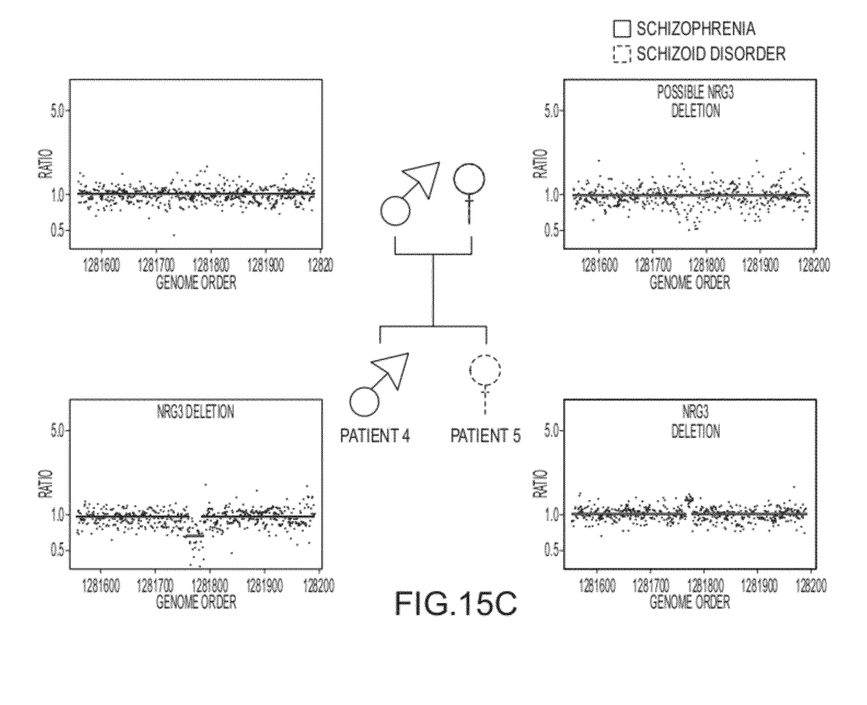

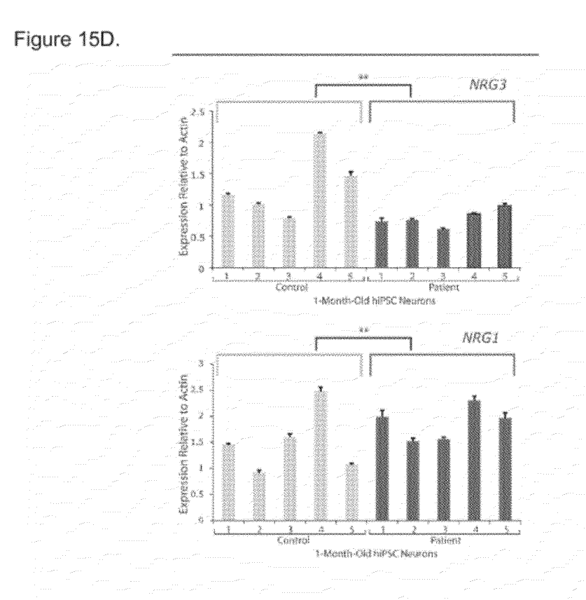

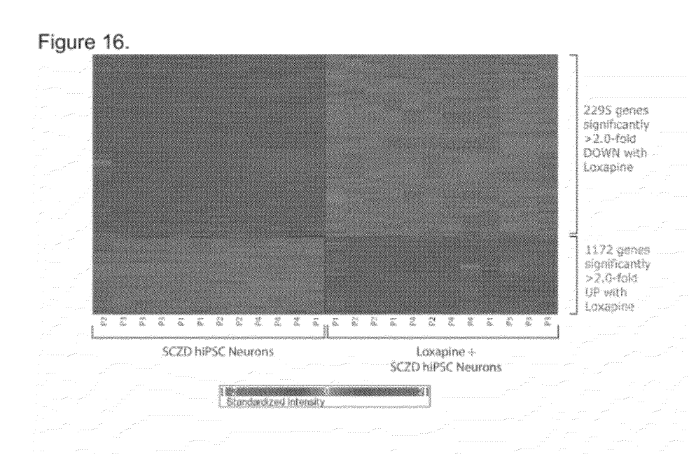

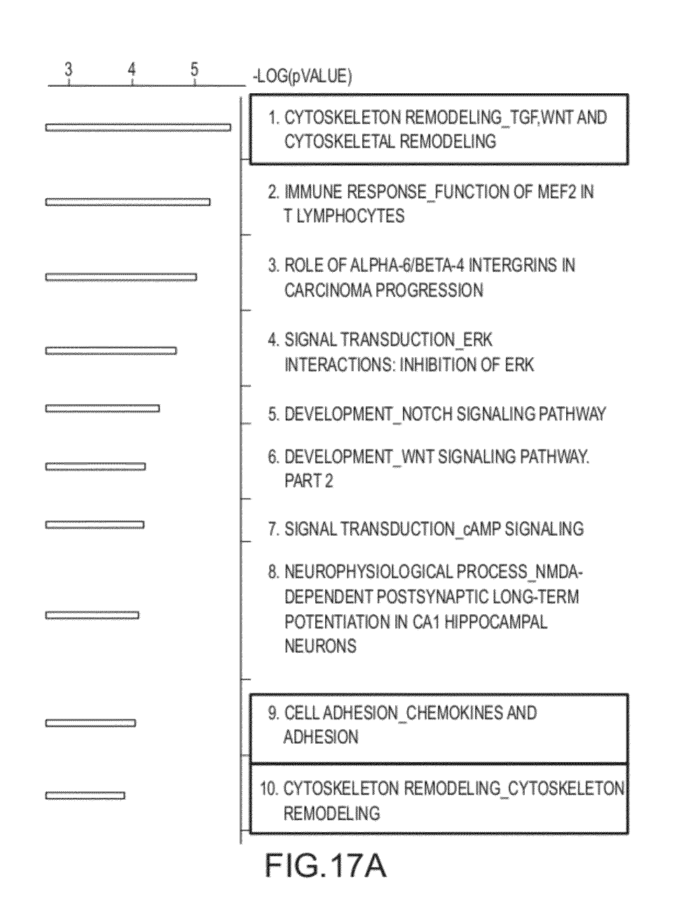

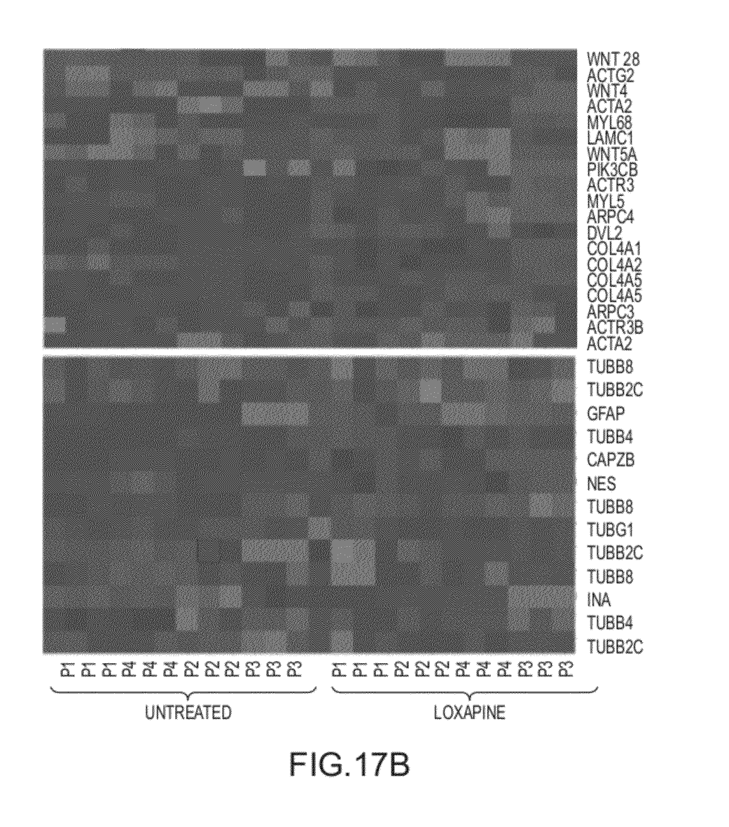

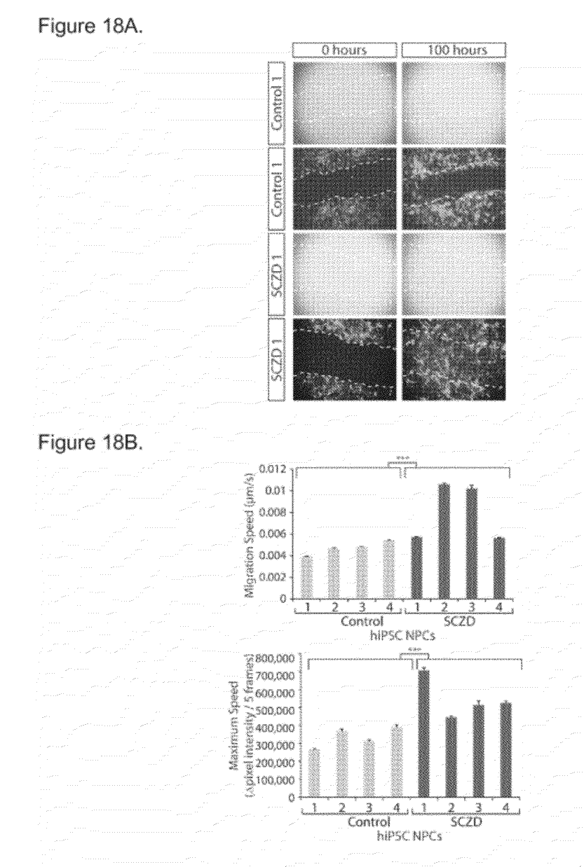

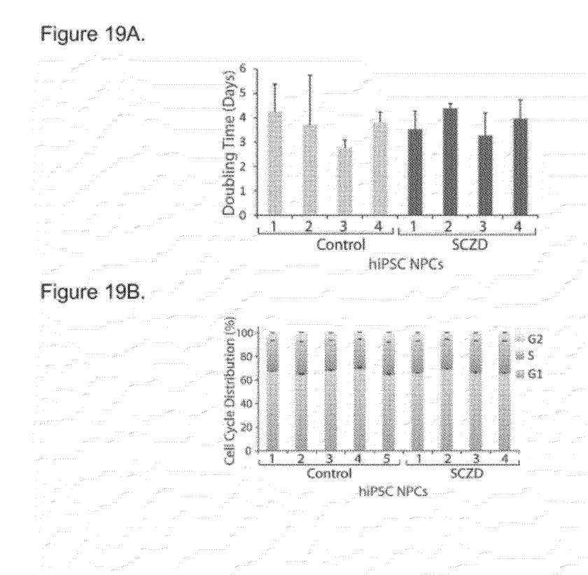

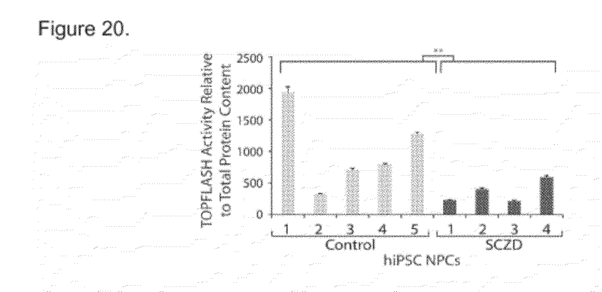

Copy number variants (CNVs) are rare, highly penetrant structural disruptions. SCZD patients have a 1.15-fold increase in CNV burden, but how this translates into illness is unknown. Patient 4 had four CNVs involving genes previously associated with SCZD or bipolar disorder (BD)^{13,18,19}; of these, neuronal expression of NRG3 and GALNT11, but not of CYP2C19 or GABARB2/GABARA6 was affected (FIG. 15, Table 7). A second analysis of CNVs unbiased by previous GWAS studies identified 42 genes affected by CNVs in the four SCZD patients (Table 7). Though twelve of these genes showed altered neuronal expression consistent with genotype (p<0.05), most changes were extremely small and only three (CSMD1, MYH1, MYH4) showed >1.3-fold effects (Table 7). Well-established SCZD CNVs occur at 1q21.1, 15q11.2, 15q13.3, 16p11.2 and 22q11.2,^{13,18,19}, but the relevant genes remain unidentified. The patients had no evidence of CNVs at these regions, and gene expression of the best candidate genes in each region, such as GJA8 (1q21.1), CYFIP1 (15q11.1), CHRFAM7A (15q13.3), PRODH (22q11.2), COMT (22q11.2) and ZDHHC8 (22q11.2)¹⁸′²⁰, was not affected in the SCZD hiPSC neurons (Table 8).

Consistent with published reports, Loxapine increased NRG1 expression in neurons²¹. Loxapine also increased expression of several glutamate receptors. ADCY8, PRKCA, WNT7A and TCF4 also showed ameliorated expression with Loxapine (FIG. 4F; FIG. 14C).

### C. Discussion

SCZD hiPSC neurons from heterogeneous patients had similar deficits, replicating some but not all aspects of the cellular and molecular phenotypes observed in post-mortem human studies and animal models (Table 3). Applicants observed decreased neuronal connectivity in SCZD hiPSC neurons, but not defects in synaptic function; this may reflect technical limitations of the synaptic activity assays. Due to the heterogeneity of the patient cohort and small sample size, the findings might not generalize to all subtypes of SCZD and the microarray comparisons of SCZD and control hiPSC neurons are necessarily preliminary. Gene expression studies of hiPSC neurons permit straightforward comparisons of antipsychotic treatments on live, genetically identical neurons from patients with known clinical treatment outcomes, eliminating many confounding variables of postmortem analysis such as treatment history, drug or alcohol abuse, and cause of death. For example, though Loxapine is characterized as a high affinity antagonist of serotonin 5-HT₂ receptors and dopamine D₁, D₂ and D₄ receptors²², treatment of SCZD hiPSC neurons resulted in altered gene expression and increased neuronal connectivity.

Of the 596 unique genes differentially expressed in the SCZD hiPSC neurons (>1.30-fold, p<0.05), 25% have been previously implicated in SCZD (Table 5). While the gene expression profiles of SCZD hiPSC neurons confirm and extend the major hypotheses generated by pharmacological and GWAS studies of SCZD, they also identify some pathways not before linked to SCZD, such as NOTCH signaling, SLIT/ROBO axon guidance, EFNA mediated axon growth, cell adhesion and transcriptional silencing (Table 6). Many of the genes most affected in SCZD hiPSC neurons belong to pathways previously associated with SCZD, though they have not yet been singled out as SCZD genes. For example, while PDE4B is a well-characterized SCZD gene, Applicants observed significant misexpression of PDE1C, PDE3A, PDE4D, PDE4DIP, PDE7B, ADCY7 and ADCY8. Additionally, though some key SCZD/BD genes, including NRG1 and ANK3, were misexpressed in all of the SCZD hiPSC neurons, many others, including ZNF804A, GABRB1, ERBB4, DISC1 and PDE4B, were aberrantly expressed in some but not all patients. The data support the “watershed model”²³ of SCZD whereby many different combinations of gene misfunction may disrupt the key pathways affected in SCZD. Applicants predict that, as the number of SCZD cases studied using hiPSC neurons increases, a diminishing number of genes will be consistently affected across the growing patient cohort; instead, evidence will accumulate that a handful of essential pathways can be disrupted in diverse ways to result in SCZD.

### IV. References

1. Sullivan, P. F., Kendler, K. S. & Neale, M. C. Schizophrenia as a complex trait: evidence from a meta-analysis of twin studies. Arch Gen Psychiatry 60, 1187-1192 (2003).
2. Wong, A. H. & Van Tol, H. H. Schizophrenia: from phenomenology to neurobiology. Neurosci Biobehav Rev 27, 269-306 (2003).
3. Javitt, D. C., Spencer, K. M., Thaker, G. K., Winterer, G. & Hajos, M. Neurophysiological biomarkers for drug development in schizophrenia. Nat Rev Drug Discov 7, 68-83 (2008).
4. Ebert, A. D. et al. Induced pluripotent stem cells from a spinal muscular atrophy patient. Nature 457, 277-280 (2009).
5. Lee, G. et al. Modelling pathogenesis and treatment of familial dysautonomia using patient-specific iPSCs. Nature 461, 402-406 (2009).
6. Marchetto, M. C. et al. A model for neural development and treatment of rett syndrome using human induced pluripotent stem cells. Cell 143, 527-539 (2010).
7. Maherali, N. et al. Directly Reprogrammed Fibroblasts Show Global Epigenetic Remodeling and Widespread Tissue Contribution. Cell Stem Cell 1, 55-70 (2007).
8. Ugolini, G. Use of rabies virus as a transneuronal tracer of neuronal connections: implications for the understanding of rabies pathogenesis. Dev Biol (Basel) 131, 493-506 (2008).
9. Wickersham, I. R. et al. Monosynaptic restriction of transsynaptic tracing from single, genetically targeted neurons. Neuron 53, 639-647 (2007).
10. Lafon, M. Rabies virus receptors. J Neurovirol 11, 82-87 (2005).
11. Selemon, L. D. & Goldman-Rakic, P. S. The reduced neuropil hypothesis: a circuit based model of schizophrenia. Biol Psychiatry 45, 17-25 (1999).
12. Jaaro-Peled, H., Ayhan, Y., Pletnikov, M. V. & Sawa, A. Review of pathological hallmarks of schizophrenia: comparison of genetic models with patients and nongenetic models. Schizophr Bull 36, 301-313 (2010).
13. Walsh, T. et al. Rare structural variants disrupt multiple genes in neurodevelopmental pathways in schizophrenia. Science (New York, N.Y. 320, 539-543 (2008).
14. Higgs, B. W., Elashoff, M., Richman, S. & Barci, B. An online database for brain disease research. BMC Genomics 7, 70 (2006).
15. Patil, S. T. et al. Activation of mGlu2/3 receptors as a new approach to treat schizophrenia: a randomized Phase 2 clinical trial. Nat Med 13, 1102-1107 (2007).
16. Patterson, S. L. et al. Some forms of cAMP-mediated long-lasting potentiation are associated with release of BDNF and nuclear translocation of phospho-MAP kinase. Neuron 32, 123-140 (2001).
17. Freyberg, Z., Ferrando, S. J. & Javitch, J. A. Roles of the Akt/GSK-3 and Wnt signaling pathways in schizophrenia and antipsychotic drug action. Am J Psychiatry 167, 388-396 (2010).
18. Stefansson, H. et al. Large recurrent microdeletions associated with schizophrenia. Nature 455, 232-236 (2008).
19. Rare chromosomal deletions and duplications increase risk of schizophrenia. Nature 455, 237-241 (2008).
20. Karayiorgou, M. & Gogos, J. A. The molecular genetics of the 22q11-associated schizophrenia. Brain Res Mol Brain Res 132, 95-104 (2004).
21. Wang, X. D., Su, Y. A., Guo, C. M., Yang, Y. & Si, T. M. Chronic antipsychotic drug administration alters the expression of neuregulin 1beta, ErbB2, ErbB3, and ErbB4 in the rat prefrontal cortex and hippocampus. Int J Neuropsychopharmacol 11, 553-561 (2008).
22. Kapur, S. et al. PET evidence that Loxapine is an equipotent blocker of 5-HT2 and D2 receptors: implications for the therapeutics of schizophrenia. Am J Psychiatry 154, 1525-1529 (1997).
23. Cannon, T. D. & Keller, M. C. Endophenotypes in the genetic analyses of mental disorders. Annu Rev Clin Psychol 2, 267-290 (2006).

### V. Tables and Supplementary Tables

TABLE 1
Genes identified by microarray analysis as showing the greatest
fold-change in expression in schizophrenic hiPSC-derived neurons.

| | | Fold-Change | |
|---|---|---|---|
| Gene Symbol | RefSeq | (SCZD vs Control) | P-value |
| PMP2 | NM_002677 | −10.3717 | 0.0203837 |
| FLJ16686 | AK304357 | −5.6709 | 0.0362611 |
| CHST9 | NM_031422 | −5.56568 | 0.000386164 |
| AQP4 | NM_001650 | −4.83804 | 0.0217381 |
| PRSS35 | NM_153362 | −4.57339 | 0.0391452 |
| HEY2 | NM_012259 | −4.40862 | 0.00365555 |
| GRIK1 | NM_175611 | −3.89439 | 0.0039328 |
| CYYR1 | NM_052954 | −3.5739 | 0.00808509 |
| PGM5 | NM_021965 | −3.56835 | 0.00378535 |
| GFAP | NM_002055 | −3.49626 | 0.0139662 |
| ASS1 | NM_000050 | 3.97469 | 0.0106259 |
| FAT4 | NM_024582 | 4.38381 | 0.00513286 |
| ZMAT4 | NM_024645 | 4.43352 | 0.00910539 |
| VGLL3 | NM_016206 | 4.55739 | 0.00346015 |
| IFITM1 | NM_003641 | 4.84429 | 0.0275079 |
| SNORD113-3 | NR_003231 | 5.03561 | 0.0434844 |
| SNORD114-3 | NR_003195 | 5.1557 | 0.0471367 |
| SERPINI1 | NM_001122752 | 5.61545 | 0.00474614 |
| PAX3 | NM_181458 | 6.13902 | 0.00580902 |
| ZIC1 | NM_003412 | 9.43152 | 0.00103707 |

TABLE 2
Microarray and qPCR validation of changes in expression of glutamate
receptor, cAMP and Wnt genes in schizophrenic hiPSC-derived neurons.

| | | Microarray Gene Expression | | qPCR Gene Expression | |
|---|---|---|---|---|---|
| | | Fold-Change | p-value | Fold-Change | p-value |
| Gene Symbol | RefSeq | (SCZD vs CNTL) | (Diagnosis) | (SCZD vs CNTL) | (Diagnosis) |
| GRIK1 | NM_175611 | −3.89 | 0.0039 | nd | nd |
| GRIK4 | NM_014619 | −1.90 | 0.0402 | nd | nd |
| GRIN1 | NM_007327 | 1.16 | 0.0448 | nd | nd |
| GRIN2A | NM_001134407 | −1.72 | 0.0421 | −4.26 | <0.0001 |
| GRM1 | NM_001114329 | 1.27 | 0.0052 | nd | nd |
| GRM7 | NM_181874 | −1.48 | 0.0190 | −2.26 | <0.0001 |
| DRD2 | NM_000795 | 1.13 | 0.0091 | nd | nd |
| GNG2 | NM_053064 | 1.20 | 0.0254 | nd | nd |
| ADCY8 | NM_001115 | −2.03 | 0.0496 | −4.90 | <0.0001 |
| ITPR2 | NM_002223 | −1.72 | 0.0081 | nd | nd |
| PDE1C | NM_005020 | 2.96 | 0.0414 | nd | nd |
| PDE3A | NM_000921 | 1.25 | 0.0452 | 1.47 | 0.0016 |
| PDE4D | NM_001104631 | 1.84 | 0.0001 | 2.18 | <0.0001 |
| PDE4DIP | NM_022359 | 1.48 | 0.0318 | 1.30 | 0.0076 |
| PDE6A | NM_000440 | −1.10 | 0.0329 | nd | nd |
| PDE7B | NM_018945 | 3.16 | 0.0114 | 3.60 | <0.0001 |
| PDE8A | NM_002605 | 1.19 | 0.0186 | −2.26 | <0.0001 |
| PDE10A | NM_006661 | 1.33 | 0.0330 | −1.46 | 0.1301 |
| PRKAR2A | NM_004157 | 1.25 | 0.0391 | nd | nd |
| PRKCA | NM_002737 | −2.41 | 0.0219 | −4.64 | <0.0001 |
| PIP5K1B | NM_003558 | 2.03 | 0.0252 | nd | nd |
| PIK3R3 | NM_003629 | 1.40 | 0.0329 | nd | nd |
| RAP1A | NM_001010935 | 1.19 | 0.0455 | −1.19 | 0.3198 |
| RAP2A | NM_021033 | −1.41 | 0.0066 | −2.15 | <0.0001 |
| WNT2B | NM_024494 | 1.41 | 0.0308 | nd | nd |
| WNT3 | NM_030753 | 1.31 | 0.0077 | nd | nd |
| WNT7A | NM_004625 | −1.62 | 0.0120 | −2.18 | <0.0001 |
| WNT7B | NM_058238 | 1.14 | 0.0069 | nd | nd |
| LRP5 | NM_002335 | −1.29 | 0.0135 | −1.32 | 0.1920 |
| AXIN2 | NM_004655 | 1.39 | 0.0191 | 2.93 | 0.0096 |
| TCF4 | NM_001083962 | 1.33 | 0.0148 | nd | nd |
| LEF1 | NM_016269 | 2.07 | 0.0496 | 2.10 | <0.0001 |

TABLE 3
Summary of positive and negative findings of cellular
phenotypes in SCZD hiPSC neurons.

| Predicted Aspects | | |
|---|---|---|
| of SCZD Cellular | Positive Findings in | Negative Findings in SCZD |
| Pathology | SCZD hiPSC Neurons | hiPSC Neurons |
| Reduced neuronal | Decreased rabies | — |
| connectivity | trans-neuronal tracing | |
| Reduced neurite | Reduced neurite | — |
| outgrowth | outgrowth | |
| Reduced synaptic | Decreased PSD95 | No change in vGLUT1, |
| protein levels | | GLUR1, VGAT or GEPH |
| Reduced synaptic | — | Not observed |
| density | | |
| Decreased synaptic | — | No change in EPSCs, iPSCs, |
| function | | or the amplitude, frequency |
| | | or spontaneity of spon- |
| | | taneous calcium transients |
| Antipsychotic | Improved neuronal | No improvement with |
| treatment | connectivity with | Clozapine (5 μM), |
| | Loxapine (10 μM) | Olanzapine (1 μM), |
| | | Risperidone (10 μM) and |
| | | Thioridazine (5 μM) |

TABLE 4
Microarray and qPCR validation of changes in expression of glutamate
receptor, cAMP and Wnt genes in SCZD hiPSC neurons.

| | | Microarray Gene Expression | | qPCR Gene Expression | |
|---|---|---|---|---|---|
| | | Fold-Change | | Fold-Change | |
| Gene Symbol | RefSeq | (SCZD vs CNTL) | p-value | (SCZD vs CNTL) | p-value |
| GRIK1 | NM_175611 | −3.9 | 0.0039 | −6.8 | <0.0001 |
| GRIK4 | NM_014619 | −1.9 | 0.0402 | nd | nd |
| GRIN2A | NM_001134407 | −1.7 | 0.0421 | −4.3 | <0.0001 |
| GRM1 | NM_001114329 | 1.3 | 0.0052 | nd | nd |
| GRM7 | NM_181874 | −1.5 | 0.0190 | −2.3 | <0.0001 |
| NRG1 | NM_013958 | 1.7 | 0.0038 | 2.8 | <0.0001 |
| ADCY7 | NM_001114 | −1.3 | 0.0052 | nd | nd |
| ADCY8 | NM_001115 | −2.0 | 0.0496 | −4.9 | <0.0001 |
| ITPR2 | NM_002223 | −1.7 | 0.0081 | nd | nd |
| PDE10A | NM_006661 | 1.3 | 0.0330 | −1.5 | 0.1301 |
| PDE1C | NM_005020 | 3.0 | 0.0414 | nd | nd |
| PDE3A | NM_000921 | 1.3 | 0.0452 | 1.5 | 0.0016 |
| PDE4D | NM_001104631 | 1.8 | 0.0001 | 2.2 | <0.0001 |
| PDE4DIP | NM_022359 | 1.5 | 0.0318 | 1.3 | 0.0076 |
| PDE7B | NM_018945 | 3.2 | 0.0114 | 3.6 | <0.0001 |
| PRKAR2A | NM_004157 | 1.3 | 0.0391 | nd | nd |
| PRKCA | NM_002737 | −2.4 | 0.0219 | −4.6 | <0.0001 |
| PRKG1 | NM_001098512 | −1.4 | 0.0112 | nd | nd |
| PIP5K1B | NM_003558 | 2.0 | 0.0252 | nd | nd |
| PTPRE | NM_006504 | −1.7 | 0.0213 | nd | nd |
| PTPRR | NM_002849 | 1.3 | 0.0487 | nd | nd |
| PIK3R3 | NM_003629 | 1.4 | 0.0329 | nd | nd |
| RAP2A | NM_021033 | −1.4 | 0.0066 | −2.1 | <0.0001 |
| WNT2B | NM_024494 | 1.4 | 0.0308 | nd | nd |
| WNT3 | NM_030753 | 1.3 | 0.0077 | nd | nd |
| WNT7A | NM_004625 | −1.6 | 0.0120 | −2.2 | <0.0001 |
| LRP5 | NM_002335 | −1.3 | 0.0135 | −1.3 | 0.1920 |
| AXIN2 | NM_004655 | 1.4 | 0.0191 | 2.9 | 0.0096 |
| TCF4 | NM_001083962 | 1.3 | 0.0148 | 2.8 | <0.0001 |
| LEF1 | NM_016269 | 2.1 | 0.0496 | 2.1 | <0.0001 |

TABLE 5
Genes identified as significantly misexpressed (p < 0.05) with >1.30-fold change in schizophrenic hiPSC neurons relative to controls.

| | | Fold- | | | Stanley Foundation SCZD Post-Mortem Microarray | | | | | | | |
|---|---|---|---|---|---|---|---|---|---|---|---|---|
| | | Change | | | Gene Expression Studies Consistent with SCZD | | | | | | | |
| | | (SCZD | | Published | hiPSC Neuron Gene Expression | | | | | | | |
| | | vs | p- | Associations with | | | | | | | Sklar- | Sklar- |
| Gene Symbol | RefSeq | Control) | value | SCZD | Altar-A | Altar-C | Bahn | Dobrin | Feinberg | Kato | A | B |
| PMP2 | NM_002677 | −10.372 | 0.020 | | | | | | | | | |
| FLJ16686 | AK304357 | −5.671 | 0.036 | | | | | | | | | |
| CHST9 | NM_031422 | −5.566 | 0.000 | | | | | | | | | |
| AQP4 | NM_001650 | −4.838 | 0.022 | Muratake, 2005 - | | | X | | | X | | |
| | | | | PMID: 16194264 | | | | | | | | |
| PRSS35 | NM_153362 | −4.573 | 0.039 | | | | | | | | | |
| HEY2 | NM_012259 | −4.409 | 0.004 | | | | | | | | | |
| GRIK1 | NM_175611 | −3.894 | 0.004 | Shibata, 2001 - | | | | | | | | |
| | | | | PMID: 11702055 | | | | | | | | |
| CYYR1 | NM_052954 | −3.574 | 0.008 | | | | | | | | | |
| PGM5 | NM_021965 | −3.568 | 0.001 | | | | | | | | | |
| GFAP | NM_002055 | −3.496 | 0.014 | Jungerius, 2007 - | | | | | | | | |
| | | | | PMID: 17893707 | | | | | | | | |
| SPP1 | NM_001040058 | −3.423 | 0.038 | Jungerius, 2007 - | | | | | | | | |
| | | | | PMID: 17893707 | | | | | | | | |
| SLC4A4 | NM_001098484 | −3.404 | 0.007 | | | | | | X | X | | |
| CATSPERB | NM_024764 | −3.279 | 0.038 | | | | | | | | | |
| PGM5P2 | NR_002836 | −3.145 | 0.002 | | | | | | | | | |
| CADPS2 | NM_017954 | −3.126 | 0.006 | | | | | | | | | |
| SPARCL1 | NM_001128310 | −3.021 | 0.026 | Kahler, 2008 - | | | | | | | | |
| | | | | PMID: 18384059 | | | | | | | | |
| TSPAN12 | NM_012338 | −2.948 | 0.001 | | | | | | | | | |
| PGM5P2 | NR_002836 | −2.943 | 0.003 | | | | | | | | | |
| FAM189A2 | NM_004816 | −2.917 | 0.007 | | | | | | | X | | |
| C1orf61 | NM_006365 | −2.897 | 0.029 | | | | | | | | | |
| LRIG3 | NM_153377 | −2.835 | 0.009 | | | | | | | | | |
| SLC34A2 | NM_006424 | −2.572 | 0.005 | | | | | | | | | |
| ARAP2 | NM_015230 | −2.514 | 0.001 | | | | | | | X | | |
| KLHDC8A | NM_018203 | −2.494 | 0.016 | | | | | | | | | |
| ADAMTS15 | NM_139055 | −2.494 | 0.014 | | | | | | | | | |
| PTCH1 | NM_001083603 | −2.420 | 0.045 | | | | | | | | | |
| C21orf63 | NM_058187 | −2.412 | 0.025 | | | | | | | | | |
| PRKCA | NM_002737 | −2.407 | 0.022 | Carroll, 2009 - | | | | | | | | |
| | | | | PMID: 19786960 | | | | | | | | |
| LRAT | NM_004744 | −2.378 | 0.041 | | | | | | | | | |
| RASGRP1 | NM_005739 | −2.355 | 0.017 | | | | | | | | | |
| SOX6 | NM_017508 | −2.344 | 0.025 | | | | | | | | | |
| MFSD2 | NM_001136493 | −2.343 | 0.008 | | | | | | | | | |
| ACTN2 | NM_001103 | −2.325 | 0.002 | | | | | | | | | |
| KCNJ16 | NM_170742 | −2.269 | 0.006 | | | | | | | | | |
| SH3GL2 | NM_003026 | −2.266 | 0.009 | Martins-de-Souza, | | | | | | | | |
| | | | | 2009 - PMID: | | | | | | | | |
| | | | | 19165527 | | | | | | | | |
| SLC44A5 | NM_152697 | −2.254 | 0.006 | | | | | | | | | |
| ASTN1 | NM_004319 | −2.245 | 0.011 | Kahler, 2008 - | | | | | | | | |
| | | | | PMID: 18384059 | | | | | | | | |
| KCNJ10 | NM_002241 | −2.236 | 0.014 | Shen, 2010 - | | | | | | | | |
| | | | | PMID: 20933057 | | | | | | | | |
| GALNT13 | NM_052917 | −2.185 | 0.013 | | | | | | | | | |
| ASCL1 | NM_004316 | −2.170 | 0.010 | Ide, 2005 - PMID: | | | | | | | X | |
| | | | | 16021468 | | | | | | | | |
| ITGA6 | NM_000210 | −2.156 | 0.010 | | | | | | | | | |
| ADHFE1 | NM_144650 | −2.139 | 0.013 | | | | | | | | | |
| SDC3 | NM_014654 | −2.135 | 0.036 | | | | | | | | | |
| GPT2 | NM_133443 | −2.131 | 0.014 | | | | | | | | | |
| SPON1 | NM_006108 | −2.112 | 0.040 | | | X | | | | X | | |
| ADCYAP1R1 | NM_001118 | −2.093 | 0.033 | Hashimoto, 2007 - | | | | | | | | |
| | | | | PMID: 17387318 | | | | | | | | |
| SLC47A2 | NM_152908 | −2.086 | 0.010 | | | | | | | | | |
| HES6 | NM_018645 | −2.063 | 0.002 | | | | | | | | | |
| ADCY8 | NM_001115 | −2.025 | 0.050 | | | | | | | | | |
| IGSF9B | NM_014987 | −2.018 | 0.010 | | | | | | | | | |
| ALDH4A1 | NM_003748 | −2.010 | 0.019 | | | | | | | | | |
| TACR1 | NM_001058 | −2.010 | 0.038 | Giegling, 2007 - | | | | | | | | |
| | | | | PMID: 17443717 | | | | | | | | |
| TC2N | NM_001128596 | −2.009 | 0.046 | | | | | | | | | |
| DBI | NM_020548 | −1.992 | 0.031 | Niu, 2004 - PMID: | | | | | | | | |
| | | | | 14755437 | | | | | | | | |
| CD34 | NM_001773 | −1.984 | 0.022 | | | | | | | | | |
| EGF | NM_001963 | −1.981 | 0.021 | Anttila, 2004 - | | | | | | | | |
| | | | | PMID: 15129177 | | | | | | | | |
| NTN1 | NM_004822 | −1.979 | 0.016 | | | | | | | | | |
| AQP7P1 | NR_002817 | −1.965 | 0.007 | | | | | | | | | |
| KCND3 | NM_004980 | −1.960 | 0.003 | | | | | | | | | |
| SERINC5 | NM_178276 | −1.959 | 0.009 | | | | | | | | | |
| SLC6A9 | NM_201649 | −1.950 | 0.023 | Deng, 2008 - | | | | | | | | |
| | | | | PMID: 18638388 | | | | | | | | |
| C10orf72 | NM_001031746 | −1.950 | 0.031 | | | | | | | | | |
| DLGAP1 | NM_004746 | −1.941 | 0.021 | Aoyama, 2003 - | | | | | | | | |
| | | | | PMID: 12950712 | | | | | | | | |
| RGMA | NM_020211 | −1.926 | 0.025 | | | | | | | | | |
| DLL1 | NM_005618 | −1.924 | 0.034 | | | | | | | | | |
| AQP7P1 | NR_002817 | −1.922 | 0.007 | | | | | | | | | |
| AQP7P1 | NR_002817 | −1.912 | 0.005 | | | | | | | | | |
| AQP7P1 | NR_002817 | −1.911 | 0.006 | | | | | | | | | |
| GRIK4 | NM_014619 | −1.904 | 0.040 | Betcheva, 2009 - | | | | | | | | |
| | | | | PMID: 19158809 | | | | | | | | |
| NOTCH1 | NM_017617 | −1.903 | 0.014 | Jungerius, 2007 - | | | | | | | | |
| | | | | PMID: 17893707 | | | | | | | | |
| HEPACAM | NM_152722 | −1.897 | 0.001 | | | | | | | | | |
| PLEKHB1 | NM_021200 | −1.896 | 0.020 | | | | | | | | | |
| ARHGEF4 | NM_032995 | −1.894 | 0.003 | | | | | | | | | |
| MAN1C1 | NM_020379 | −1.884 | 0.003 | | | | | | | | | |
| C4A | NM_007293 | −1.881 | 0.001 | Rudduck, 1985 - | | | | X | | X | | |
| | | | | PMID: 3875548 | | | | | | | | |
| LOC283174 | NR_024344 | −1.876 | 0.007 | | | | | | | | | |
| PRDM8 | NM_020226 | −1.876 | 0.001 | | | | | | | | | |
| ITGA7 | NM_001144996 | −1.868 | 0.035 | | | | | | | | | |
| IGSF11 | NM_001015887 | −1.867 | 0.023 | | | | | | | | | |
| PFKFB3 | NM_004566 | −1.860 | 0.031 | | | | | | | | | |
| SLC38A3 | NM_006841 | −1.845 | 0.015 | | | | | | | | | |
| SGEF | NM_015595 | −1.842 | 0.009 | | | | | | | | | |
| LYPD6 | NM_194317 | −1.815 | 0.005 | | | | | | | | | |
| STAC | NM_003149 | −1.814 | 0.014 | | | | | | | | | |
| VWA5A | NM_001130142 | −1.814 | 0.007 | | | | | | | | | |
| S100A16 | NM_080388 | −1.810 | 0.001 | | | | | | | | | |
| IGSF9B | NM_014987 | −1.810 | 0.006 | | | | | | | | | |
| NKX6-1 | NM_006168 | −1.807 | 0.002 | | | | | | | | | |
| DDIT4L | NM_145244 | −1.792 | 0.001 | | | | | | | | | |
| MGST1 | NM_145792 | −1.791 | 0.019 | | | | | | | | | |
| ALDH1L1 | NM_012190 | −1.782 | 0.000 | Kurian, 2011 - | X | | X | | | X | | |
| | | | | PMID: 19935739 | | | | | | | | |
| RNF148 | NM_198085 | −1.773 | 0.021 | | | | | | | | | |
| ATP1B2 | NM_001678 | −1.770 | 0.021 | | | | | | | | | |
| LPAR6 | NM_005767 | −1.766 | 0.025 | | X | | | | | | | |
| MEGF10 | NM_032446 | −1.765 | 0.024 | Chen, 2008 - | | | | | | | | |
| | | | | PMID: 18179784 | | | | | | | | |
| SLC39A12 | NM_001145195 | −1.758 | 0.004 | | | | | | | | | |
| ALDOC | NM_005165 | −1.757 | 0.040 | Martins-de-Souza, | | | | | | | | |
| | | | | 2008 - PMID: | | | | | | | | |
| | | | | 19110265 | | | | | | | | |
| ANKFN1 | NM_153228 | −1.754 | 0.042 | | | | | | | | | |
| RNF133 | NM_139175 | −1.754 | 0.044 | | | | | | | | | |
| NOG | NM_005450 | −1.751 | 0.003 | | | | | | | | | |
| LRRCC1 | NM_033402 | −1.751 | 0.006 | | | | | | | | | |
| ACSBG1 | NM_015162 | −1.746 | 0.012 | | | | | | | X | | |
| WBSCR17 | NM_022479 | −1.746 | 0.027 | | | | | | | | | |
| ITGA3 | NM_002204 | −1.740 | 0.017 | Kahler, 2008 - | | | | | | | | |
| | | | | PMID: 18384059 | | | | | | | | |
| PSRC1 | NM_001032290 | −1.738 | 0.002 | | | | X | X | | | | |
| ROBO2 | NM_002942 | −1.737 | 0.028 | | | | | | | | | |
| CCDC144A | NM_014695 | −1.736 | 0.026 | | | | | | | | | |
| ZBTB16 | NM_006006 | −1.735 | 0.002 | | | | | | X | | | |
| EFHD1 | NM_025202 | −1.732 | 0.028 | | | | | | X | | | |
| GRIN2A | NM_001134407 | −1.725 | 0.042 | Itokawa, 2003 - | | | | | | | | |
| | | | | PMID: 12724619 | | | | | | | | |
| SOX2 | NM_003106 | −1.720 | 0.048 | | | | | | | | | |
| ITPR2 | NM_002223 | −1.720 | 0.008 | | | | | X | | X | | |
| NCAM1 | NM_181351 | −1.714 | 0.046 | Atz, 2007 - PMID: | | | | | | | X | |
| | | | | 17413444 | | | | | | | | |
| SCARA3 | NM_016240 | −1.711 | 0.022 | | | | | | | | | |
| RAB31 | NM_006868 | −1.711 | 0.014 | | | | X | | | X | | X |
| SAMD4A | NM_015589 | −1.703 | 0.018 | | | | | | | | | |
| ZDHHC14 | NM_024630 | −1.702 | 0.008 | | | | | | | | | |
| PTPRE | NM_006504 | −1.702 | 0.021 | | | | | | | | | |
| S1PR1 | NM_001400 | −1.701 | 0.044 | | | | | | | X | | |
| SLC35D2 | NM_007001 | −1.700 | 0.037 | | | | | | | | | |
| LAMA5 | NM_005560 | −1.692 | 0.001 | | | | | | | | | |
| C4orf22 | BC034296 | −1.691 | 0.033 | | | | | | | | | |
| FAM181B | NM_175885 | −1.690 | 0.003 | | | | | | | | | |
| CNKSR3 | NM_173515 | −1.688 | 0.007 | | | | | | X | | | |
| SLC30A10 | NM_018713 | −1.687 | 0.028 | | | | | | | | | |
| C18orf16 | AK055069 | −1.684 | 0.012 | | | | | | | | | |
| KCNA4 | NM_002233 | −1.681 | 0.004 | | | | | | | | | |
| CSPG5 | NM_006574 | −1.678 | 0.046 | So, 2009 - PMID: | | X | | | | | | |
| | | | | 19367581 | | | | | | | | |
| SLC12A4 | NM_005072 | −1.673 | 0.005 | | | | | | | | | |
| HNMT | NM_006895 | −1.672 | 0.001 | Yan, 2000 - | | | | | | | | |
| | | | | PMID: 10898922 | | | | | | | | |
| CAV2 | NM_001233 | −1.662 | 0.032 | | | | | | | | | |
| EDNRB | NM_001122659 | −1.661 | 0.030 | | | | | | | X | | |
| UCP2 | NM_003355 | −1.660 | 0.000 | Yasuno, 2006 - | | | | | | | | |
| | | | | PMID: 17066476 | | | | | | | | |
| CTD- | NR_004846 | −1.656 | 0.028 | | | | | | | | | |
| 2514C3.1 | | | | | | | | | | | | |
| PRCP | NM_199418 | −1.653 | 0.000 | | | | | | | | | |
| CYTSB | NM_001033553 | −1.651 | 0.050 | | | | | | | | | |
| SYNM | NM_145728 | −1.651 | 0.006 | | | | | | | | | |
| NEXN | NM_144573 | −1.649 | 0.014 | | | | | | | | | |
| NAT8L | NM_178557 | −1.645 | 0.003 | | | | | | | | | |
| LIX1 | NM_153234 | −1.642 | 0.016 | | | | | | | | | |
| TMEM132A | NM_017870 | −1.641 | 0.010 | | | | | | | | | |
| LRP1B | NM_018557 | −1.636 | 0.041 | | | | | | | | | |
| TLE3 | NM_005078 | −1.636 | 0.001 | | | | | | | | | |
| ITGB4 | NM_000213 | −1.635 | 0.041 | | | | X | | | X | | |
| SEMA5B | NM_001031702 | −1.630 | 0.043 | | | | | | | | | |
| CCDC144A | NM_014695 | −1.627 | 0.022 | | | | | | | | | |
| WNT7A | NM_004625 | −1.623 | 0.012 | | | | | | | | | |
| CELSR2 | NM_001408 | −1.622 | 0.035 | | | | | | | | X | |
| TPST1 | NM_003596 | −1.617 | 0.037 | | | | | | | | | |
| UNQ9374 | AY358216 | −1.615 | 0.012 | | | | | | | | | |
| AK3L1 | NM_001005353 | −1.607 | 0.011 | | | | | | | | | |
| ADRA1A | NM_000680 | −1.603 | 0.039 | Bolonna, 2000 - | | | | | | | | |
| | | | | PMID: 10696813 | | | | | | | | |
| FMNL2 | NM_052905 | −1.597 | 0.013 | | | | | X | | | | |
| PRPH | NM_006262 | −1.590 | 0.024 | | | | | | | | | |
| ODZ4 | NM_001098816 | −1.584 | 0.019 | | | | | | | | | |
| FAM84B | NM_174911 | −1.579 | 0.024 | | | | | | | | | |
| ARNTL2 | NM_020183 | −1.578 | 0.040 | Mansour, 2009 - | | | | | | | | |
| | | | | PMID: 19839995 | | | | | | | | |
| TYRO3 | NM_006293 | −1.578 | 0.003 | | | | | | X | | | |
| RNF220 | NM_018150 | −1.574 | 0.006 | | | | | | | | | |
| LAP3 | NM_015907 | −1.574 | 0.016 | | | | | | | | | |
| TRIM68 | NM_018073 | −1.569 | 0.043 | | | | | | | | | |
| COBL | NM_015198 | −1.567 | 0.015 | | | | | | | | | |
| SYTL2 | NM_206927 | −1.566 | 0.021 | | | | | | | | | |
| VAMP1 | NM_199245 | −1.563 | 0.004 | | | | | | | X | | |
| RXRG | NM_006917 | −1.563 | 0.025 | | | | | | | | | |
| CD9 | NM_001769 | −1.562 | 0.041 | | | | | | | | X | |
| SOCS2 | NM_003877 | −1.562 | 0.038 | | | | | | | | | |
| KAT2B | NM_003884 | −1.561 | 0.024 | | | | | | X | | | |
| MARCH3 | NM_178450 | −1.553 | 0.023 | | | | | | | | | |
| PPP2R2B | NM_004576 | −1.549 | 0.041 | Chen, 2005 - | | X | | | | | | |
| | | | | PMID: 16054804 | | | | | | | | |
| TAAR3 | BC095548 | −1.548 | 0.004 | | | | | | | | | |
| DNHD1 | NM_144666 | −1.547 | 0.049 | | | | | | | | | |
| ABCD2 | NM_005164 | −1.546 | 0.041 | | | | | | | | | |
| PRAGMIN | NM_001080826 | −1.542 | 0.017 | | | | | | | | | |
| B4GALT5 | NM_004776 | −1.541 | 0.015 | | | | | | | | | |
| FJX1 | NM_014344 | −1.534 | 0.036 | | | | | | | | | |
| MYH2 | NM_017534 | −1.524 | 0.019 | | | | | | | | | |
| ATG4C | NM_032852 | −1.522 | 0.034 | | | | | | | | | |
| KCNMB1 | NM_004137 | −1.520 | 0.022 | | | | | | | | | |
| CAT | NM_001752 | −1.517 | 0.033 | | | | | | | | | |
| ADAM32 | NM_145004 | −1.516 | 0.020 | | | | | | | | | |
| PGM5 | NM_021965 | −1.508 | 0.004 | | | | | | | | | |
| NOD1 | NM_006092 | −1.505 | 0.004 | | | | | | | | | |
| KALRN | NM_001024660 | −1.497 | 0.047 | | | | | | | | | |
| FAM106A | NR_026809 | −1.494 | 0.033 | | | | | | | | | |
| TTYH2 | NM_032646 | −1.493 | 0.044 | | | | | | | | | |
| MFHAS1 | NM_004225 | −1.488 | 0.013 | | | | | | | | | |
| GRM7 | NM_181874 | −1.479 | 0.019 | Bolonna, 2001 - | | | | | | | | |
| | | | | PMID: 11163549 | | | | | | | | |
| RASL10A | NM_001007279 | −1.478 | 0.023 | Saito, 2010 - | | | | | | | | |
| | | | | PMID: 20537721 | | | | | | | | |
| GMPR | NM_006877 | −1.477 | 0.024 | Lin, 2009 - PMID: | | | | | | | | |
| | | | | 19694819 | | | | | | | | |
| CRISPLD1 | NM_031461 | −1.471 | 0.022 | | | | | | | | | |
| TAS2R4 | NM_016944 | −1.468 | 0.009 | | | | | | | | | |
| THUMPD2 | NM_025264 | −1.467 | 0.003 | | | | | | | | | |
| LFNG | NM_001040167 | −1.466 | 0.013 | | | | | | | | | |
| PPP2R5A | NM_006243 | −1.466 | 0.000 | | | | | | | | | |
| LOC162632 | NR_003190 | −1.466 | 0.033 | | | | | | | | | |
| URB1 | NM_014825 | −1.464 | 0.007 | | | | | | | | | |
| RASA4 | NM_006989 | −1.462 | 0.024 | | | | | | | | | |
| FLJ16734 | AK131514 | −1.461 | 0.050 | | | | | | | | | |
| C1orf62 | NM_152763 | −1.453 | 0.048 | | | | | | | | | |
| GLIPR2 | NM_022343 | −1.452 | 0.012 | | | | | | | | | |
| RAB7L1 | NM_003929 | −1.450 | 0.025 | | | | | | X | | | |
| IMMP2L | NM_032549 | −1.448 | 0.002 | | | | | | | | | |
| IFIT2 | NM_001547 | −1.447 | 0.002 | | | | | | | | | |
| NDST3 | NM_004784 | −1.445 | 0.014 | | | | | | | | | |
| WASF2 | NM_006990 | −1.445 | 0.000 | | | | | | | | | |
| FAM106A | NR_026809 | −1.445 | 0.029 | | | | | | | | | |
| SORD | NM_003104 | −1.444 | 0.004 | | | | | | | | | |
| B3GALT5 | NM_033171 | −1.442 | 0.038 | | | | | | | | | |
| GRHL1 | NM_198182 | −1.440 | 0.009 | | | | | | | | | |
| FBXL3 | NM_012158 | −1.439 | 0.006 | | | | | | | | | |
| CACNG7 | NM_031896 | −1.439 | 0.024 | | | | | | | | | |
| AQP7 | NM_001170 | −1.436 | 0.002 | | | | | | | | | |
| QDPR | NM_000320 | −1.435 | 0.049 | | | X | | | | | | |
| ABLIM1 | NM_002313 | −1.433 | 0.041 | | | | | | | | | |
| TAF1C | NM_005679 | −1.433 | 0.005 | | | | | | | | | |
| BAG3 | NM_004281 | −1.431 | 0.025 | Ikeda, 2009 - | | | | | | | | |
| | | | | PMID: 19850283 | | | | | | | | |
| RNASEH2B | NM_024570 | −1.430 | 0.011 | | | | | | | | | |
| SNORA49 | NR_002979 | −1.429 | 0.017 | | | | | | | | | |
| PHF17 | NM_199320 | −1.427 | 0.032 | | | | | | | | | |
| C13orf38 | NM_001144981 | −1.426 | 0.008 | | | | | | | | | |
| C9orf21 | NM_153698 | −1.423 | 0.012 | | | | | | | | | |
| HIST1H4D | NM_003539 | −1.423 | 0.009 | | | | | | | | X | |
| FAM106A | NR_026809 | −1.422 | 0.023 | | | | | | | | | |
| PBK | NM_018492 | −1.422 | 0.041 | | | | | | | | | |
| GNPDA1 | NM_005471 | −1.420 | 0.042 | | | X | | | | | | |
| LRRC61 | NM_001142928 | −1.420 | 0.018 | | | | | | | | | |
| PRKG1 | NM_001098512 | −1.417 | 0.011 | | | | | | | | | |
| F2R | NM_001992 | −1.416 | 0.022 | | | | | | | | | |
| RHPN1 | NM_052924 | −1.416 | 0.031 | | | | | | | | | |
| RAP2A | NM_021033 | −1.414 | 0.007 | | | | | | | | | |
| MST1 | NM_020998 | −1.413 | 0.015 | | | | | | | | | |
| DYNC1LI2 | NM_006141 | −1.413 | 0.027 | | | | | | | | X | |
| PLCD1 | NM_006225 | −1.412 | 0.007 | | | | | | | | | |
| FUT8 | NM_178155 | −1.412 | 0.040 | | | | | | | | | |
| SPTAN1 | NM_001130438 | −1.411 | 0.033 | Murakami, 1999 - | | | | | | | | |
| | | | | PMID: 10402491 | | | | | | | | |
| FGFR3 | NM_000142 | −1.409 | 0.008 | Jungerius, 2007 - | | | X | | | | | |
| | | | | PMID: 17893707 | | | | | | | | |
| PITPNC1 | NM_181671 | −1.409 | 0.042 | | | | | | | | | |
| CA13 | NM_198584 | −1.407 | 0.031 | | | | | | | | | |
| EZR | NM_003379 | −1.407 | 0.029 | | | | | | | | | |
| MGC13005 | NR_024005 | −1.403 | 0.015 | | | | | | | | | |
| DCHS2 | NM_017639 | −1.402 | 0.018 | | | | | | | | | |
| SLC44A1 | NM_080546 | −1.401 | 0.034 | | | | | | | | | |
| DNHD1 | NM_173589 | −1.400 | 0.024 | | | | | | | | | |
| C8orf79 | NM_020844 | −1.396 | 0.046 | | | | | | | | | |
| TMEM185B | NR_000034 | −1.395 | 0.008 | | | | | | | | | |
| SNORA41 | NR_002590 | −1.395 | 0.013 | | | | | | | | | |
| CHST15 | NM_015892 | −1.394 | 0.024 | | | | | | | | | |
| CCDC144C | NR_023380 | −1.394 | 0.026 | | | | | | | | | |
| UBQLN4 | NM_020131 | −1.394 | 0.043 | | | | | | | | | |
| SEMA4B | NM_020210 | −1.393 | 0.048 | | | | | | | | | |
| C1orf183 | NM_019099 | −1.393 | 0.029 | | | | | | | | | |
| NPNT | NM_001033047 | −1.391 | 0.001 | | | | | | | | | |
| ARID5A | NM_212481 | −1.391 | 0.014 | | | | | | | | | |
| WWP1 | NM_007013 | −1.386 | 0.004 | | | | | | | | | |
| PAQR6 | NM_024897 | −1.385 | 0.000 | | | | | | | | | |
| REV3L | NM_002912 | −1.385 | 0.047 | | | | | | | | | |
| SNORD52 | NR_002742 | −1.385 | 0.033 | | | | | | | | | |
| KAT2A | NM_021078 | −1.380 | 0.013 | | | | | | | | X | |
| BAI1 | NM_001702 | −1.378 | 0.014 | | | | | | | | | |
| TRAF3IP2 | NM_147686 | −1.377 | 0.022 | | | | | | | | | |
| CEP78 | NM_001098802 | −1.377 | 0.000 | | | | | | | | | |
| EGLN3 | NM_022073 | −1.375 | 0.002 | | | | | | | | | |
| FGD3 | NM_001083536 | −1.372 | 0.003 | | | | | | | | | |
| MYBL1 | NM_001080416 | −1.372 | 0.028 | | | | | | | | | |
| DLEU2 | NR_002612 | −1.372 | 0.047 | | | | | | | | | |
| ALDH7A1 | NM_001182 | −1.371 | 0.014 | | | | X | | | X | | |
| BLVRA | NM_000712 | −1.368 | 0.003 | | | | | | | | | |
| LCAT | NM_000229 | −1.367 | 0.001 | | | | | | | | | |
| LCTL | NM_207338 | −1.367 | 0.024 | | | | | | | | | |
| FABP4 | NM_001442 | −1.366 | 0.008 | | | | | | | | | |
| DOCK7 | NM_033407 | −1.364 | 0.021 | | | | | | | | | |
| CDK5RAP2 | NM_018249 | −1.363 | 0.014 | | | | | | | | | |
| SOBP | NM_018013 | −1.362 | 0.042 | | | | | | | | | |
| SEMA4C | NM_017789 | −1.362 | 0.044 | | | | | | | | | |
| MSTP9 | NR_002729 | −1.362 | 0.017 | | | | | | | | | |
| C16orf93 | NM_001014979 | −1.360 | 0.034 | | | | | | | | | |
| CLN8 | NM_018941 | −1.359 | 0.011 | | | | | | | | | |
| METTL7A | NM_014033 | −1.358 | 0.045 | | | | | | | X | | |
| CLN8 | NM_018941 | −1.358 | 0.028 | | | | | | | | | |
| SGCG | NM_000231 | −1.357 | 0.030 | | | | | | | | | |
| ESCO2 | NM_001017420 | −1.356 | 0.023 | | | | | | | | | |
| TANC1 | NM_033394 | −1.356 | 0.041 | | | | | | | | | |
| SPTB | NM_001024858 | −1.355 | 0.013 | | | | | | | | | |
| SEPN1 | NM_020451 | −1.354 | 0.047 | | | | | | | | | |
| KIAA1618 | NM_020954 | −1.354 | 0.006 | | | | | | | | | |
| ERI1 | NM_153332 | −1.354 | 0.029 | | | | | | | | | |
| LOC400464 | AK127420 | −1.351 | 0.014 | | | | | | | | | |
| THBS4 | NM_003248 | −1.348 | 0.001 | | | | | | | | | |
| RNF213 | NM_020914 | −1.347 | 0.029 | | | | | X | | | | |
| SLC35D1 | NM_015139 | −1.347 | 0.011 | | | | | | | | | |
| SNRPG | NM_003096 | −1.346 | 0.002 | | | | | | | | | |
| GPR37L1 | NM_004767 | −1.345 | 0.014 | | | | | | | | | |
| GAL3ST4 | NM_024637 | −1.343 | 0.037 | | | | | | | | | |
| FAM53B | NM_014661 | −1.343 | 0.035 | | | | | | | | | |
| LSM6 | NM_007080 | −1.340 | 0.013 | | | | | | | | | |
| ZFP36L1 | NM_004926 | −1.339 | 0.006 | | | | | | | | | |
| MAML2 | NM_032427 | −1.338 | 0.031 | | | | | | | | | |
| SNORD109A | NR_001295 | −1.336 | 0.037 | | | | | | | | | |
| SNORD109A | NR_001295 | −1.336 | 0.037 | | | | | | | | | |
| TM7SF2 | NM_003273 | −1.333 | 0.037 | | | | | | | | X | |
| UNQ9370 | AY358254 | −1.333 | 0.016 | | | | | | | | | |
| PINK1 | NM_032409 | −1.331 | 0.010 | Funayama, 2008 - | | | X | | | | | |
| | | | | PMID: 18546294 | | | | | | | | |
| GPR155 | NM_001033045 | −1.330 | 0.031 | | | | | | | | | |
| TST | NM_003312 | −1.330 | 0.019 | | | X | X | | | X | | |
| ZNF436 | NM_001077195 | −1.328 | 0.031 | | | | | | | | | |
| LRRN2 | NM_006338 | −1.327 | 0.048 | | | | | | | | | |
| TCEA3 | NM_003196 | −1.327 | 0.049 | | | | | | | | | |
| NHSL1 | AK299585 | −1.326 | 0.034 | | | | | | | | | |
| FAM182A | NR_026713 | −1.323 | 0.010 | | | | | | | | | |
| ARL6IP6 | NM_152522 | −1.321 | 0.035 | | | | | | | | | |
| SCARNA17 | NR_003003 | −1.317 | 0.006 | | | | | | | | | |
| NBEA | NM_015678 | −1.317 | 0.032 | | | | X | | | | | |
| FAM182A | NR_026713 | −1.316 | 0.011 | | | | | | | | | |
| DNHD1 | NM_144666 | −1.314 | 0.014 | | | | | | | | | |
| PIK3IP1 | NM_052880 | −1.314 | 0.013 | | | | | | | | | |
| FNTB | NM_002028 | −1.313 | 0.001 | | | | | | | | | |
| CNN3 | NM_001839 | −1.313 | 0.046 | | | | | | | | | |
| CENPQ | NM_018132 | −1.313 | 0.022 | | | | | | | | | |
| COQ2 | NM_015697 | −1.311 | 0.004 | | | | | X | | | | |
| ADCY7 | NM_001114 | −1.310 | 0.005 | | | | | | | | | |
| PSENEN | NM_172341 | −1.310 | 0.009 | | | | | | | | | |
| LOC440957 | NM_001124767 | −1.310 | 0.035 | | | | | | | | | |
| NACC2 | NM_144653 | −1.308 | 0.032 | | | | | | | | | |
| FAM181A | BC009073 | −1.308 | 0.022 | | | | | | | | | |
| C14orf149 | NM_144581 | −1.308 | 0.028 | | | | | | | | | |
| DAG1 | NM_004393 | −1.307 | 0.013 | | | | | | | | | |
| HSD17B3 | NM_000197 | −1.307 | 0.002 | | | | | | | | | |
| FLJ00049 | AK024457 | −1.306 | 0.007 | | | | | | | | | |
| VASH1 | NM_014909 | −1.306 | 0.028 | | | | | | | | | |
| NR2F1 | NM_005654 | −1.305 | 0.029 | | | | | | | | X | |
| MAN2A2 | NM_006122 | −1.305 | 0.003 | | | X | | | | | | |
| KCNG4 | NM_172347 | −1.302 | 0.006 | | | | | | | | | |
| OR2J3 | NM_001005216 | −1.302 | 0.042 | | | | | | | | | |
| FAM168A | EF363480 | −1.301 | 0.023 | | | X | | | | | | |
| FAM3C | NM_014888 | −1.301 | 0.021 | | | | | | | | | |
| SH3KBP1 | AY423734 | −1.301 | 0.010 | | | | | | | | | |
| C17orf61 | BC030270 | −1.300 | 0.015 | | | | | | | | | |
| ARHGEF17 | NM_014786 | −1.300 | 0.039 | | | | | | | | | |
| SLC25A12 | NM_003705 | 1.300 | 0.026 | Hong, 2007 - | | X | | | | | | |
| | | | | PMID: 17693006 | | | | | | | | |
| KIAA0802 | BC040542 | 1.300 | 0.018 | | | | | | | | | |
| CPD | NM_001304 | 1.301 | 0.030 | | | | | | | | X | |
| MKNK2 | NM_199054 | 1.301 | 0.008 | | | | | | | | | |
| PRICKLE2 | NM_198859 | 1.301 | 0.006 | | | | | | | | | |
| SESN1 | NM_014454 | 1.304 | 0.026 | | | | | X | | | | |
| FOXN3 | NM_001085471 | 1.304 | 0.004 | | | X | | | | | | |
| HIVEP1 | NM_002114 | 1.304 | 0.000 | | | | | | | | | |
| SLAIN2 | NM_020846 | 1.306 | 0.005 | | | | | X | | | | |
| KREMEN1 | NM_001039570 | 1.306 | 0.022 | Aleksic, 2010 - | | | | | | | | |
| | | | | PMID: 20153141 | | | | | | | | |
| ADAMTSL1 | NM_001040272 | 1.306 | 0.004 | | | | | | | | | |
| PNOC | NM_006228 | 1.307 | 0.001 | Blaveri, 2001 - | | | | | | X | | |
| | | | | PMID: 11436130 | | | | | | | | |
| WNT3 | NM_030753 | 1.308 | 0.008 | | | | | | | | | |
| AP2M1 | NM_004068 | 1.310 | 0.018 | | | | | | | | | |
| ST7 | NM_018412 | 1.311 | 0.024 | | | | | | | | | |
| FAM35A | NM_019054 | 1.312 | 0.032 | | | | | | | | | |
| ZNF618 | NM_133374 | 1.312 | 0.015 | | | | | | | | | |
| FLJ13197 | NR_026804 | 1.313 | 0.005 | | X | | | | | | | |
| LRP4 | NM_002334 | 1.315 | 0.046 | | | | | | | | | |
| C9orf125 | BC033550 | 1.317 | 0.039 | | | | | | | | | |
| FAM35A | NM_019054 | 1.318 | 0.028 | | | | | | | | | |
| EIF2A | NM_032025 | 1.320 | 0.005 | | | | | | | | | |
| SBNO1 | NM_018183 | 1.320 | 0.042 | | | | | | | | | |
| KLF12 | NM_007249 | 1.321 | 0.039 | | | | | | | | | |
| ZNF124 | NM_003431 | 1.323 | 0.023 | | | | | | | | | |
| SMYD3 | NM_022743 | 1.323 | 0.014 | | | X | | | | | | |
| KLHL29 | BC015667 | 1.324 | 0.001 | | | | | | | | | |
| AKR1C3 | NM_003739 | 1.326 | 0.036 | | | | | | | | | |
| ZNF217 | NM_006526 | 1.326 | 0.022 | | | | | | | | | |
| PDE10A | NM_006661 | 1.326 | 0.033 | | | | | | | | | |
| TCF4 | NM_001083962 | 1.330 | 0.015 | Bowen, 2000 - | X | X | | | | | | |
| | | | | PMID: 10909126 | | | | | | | | |
| AMOTL2 | NM_016201 | 1.333 | 0.044 | | | | | | | | X | |
| RHOBTB1 | NR_024556 | 1.333 | 0.023 | | | | | | | | | |
| POLB | NM_002690 | 1.333 | 0.038 | | | X | | | | | | |
| ZNF616 | NM_178523 | 1.334 | 0.045 | | | | | | | | | |
| SEC31A | NM_014933 | 1.335 | 0.042 | | | | | | | | X | |
| SMC5 | NM_015110 | 1.338 | 0.034 | | | | | | | | | |
| ADORA2B | NM_000676 | 1.339 | 0.003 | | | | | | | X | | |
| BOC | NM_033254 | 1.342 | 0.004 | | | | | | | | | |
| KCTD10 | NM_031954 | 1.344 | 0.011 | | | | | X | | | | |
| SPRY4 | NM_030964 | 1.347 | 0.031 | Zaharieva, 2008 - | | | | | | | | |
| | | | | PMID: 18298822 | | | | | | | | |
| NTF3 | NM_002527 | 1.349 | 0.026 | Arinami, 1996 - | | | | | | | | |
| | | | | PMID: 8925252 | | | | | | | | |
| NUP93 | NM_014669 | 1.356 | 0.022 | | | | | | | | | |
| CNGA3 | NM_001298 | 1.361 | 0.023 | | | | | | | | | |
| KLF9 | NM_001206 | 1.361 | 0.004 | | | | | | | | | |
| AUTS2 | NM_015570 | 1.361 | 0.007 | | | | | | | | | |
| FARP1 | NM_005766 | 1.363 | 0.043 | | | | | | | | | |
| P2RX3 | NM_002559 | 1.364 | 0.024 | | | | | | | | | |
| HS3ST3A1 | NM_006042 | 1.365 | 0.032 | | | | | | | | | |
| ZNF616 | BC032805 | 1.369 | 0.038 | | | | | | | | | |
| CNTN2 | NM_005076 | 1.372 | 0.046 | Jungerius, 2007 - | | | | | | | | |
| | | | | PMID: 17893707 | | | | | | | | |
| SNORD36B | NR_000017 | 1.376 | 0.016 | | | | | | | | | |
| STK24 | NM_003576 | 1.376 | 0.007 | | | | | X | | | | |
| UNQ3028 | AY358789 | 1.377 | 0.010 | | | | | | | | | |
| C17orf75 | NM_022344 | 1.378 | 0.037 | | | X | | | | | | |
| ZIC5 | NM_033132 | 1.378 | 0.005 | | | | | | | | | |
| CASP1 | NM_033292 | 1.382 | 0.004 | | | | | | | | | |
| FAM19A4 | NM_182522 | 1.383 | 0.034 | | | | | | | | | |
| EIF4B | NM_001417 | 1.385 | 0.005 | | | | | | | | | |
| AXIN2 | NM_004655 | 1.385 | 0.019 | | | | | | | | | |
| PDP1 | NM_001161778 | 1.386 | 0.016 | | | | | | | | | |
| FRMD4A | NM_018027 | 1.386 | 0.037 | | | | | | | | | |
| SLMO2 | NM_016045 | 1.386 | 0.017 | | | | | | | | | |
| INADL | NM_176877 | 1.388 | 0.034 | | | | | | X | | | |
| RPL21 | NM_000982 | 1.389 | 0.019 | | | | | | | | X | |
| KIRREL3 | NM_032531 | 1.391 | 0.003 | | | | | | | | | |
| TNFAIP3 | NM_006290 | 1.392 | 0.009 | | | | | | X | | | |
| GPR137C | NM_001099652 | 1.395 | 0.041 | | | | | | | | | |
| SCPEP1 | NM_021626 | 1.396 | 0.011 | | | | | | | | | |
| CRTC3 | NM_022769 | 1.400 | 0.001 | | | | | | | | | |
| PIK3R3 | NM_003629 | 1.403 | 0.033 | | | | | | | | | |
| ZNF823 | NM_001080493 | 1.405 | 0.004 | | | | | | | | | |
| WNT2B | NM_024494 | 1.407 | 0.031 | Proitsi, 2008 - | | | | | | | | |
| | | | | PMID: 17553464 | | | | | | | | |
| GAS2L3 | NM_174942 | 1.409 | 0.009 | | | | | | | | | |
| DGKE | NM_003647 | 1.412 | 0.025 | | | | | | | | | |
| TSC22D2 | NM_014779 | 1.412 | 0.002 | | | | | | | | | |
| ZNF583 | NM_152478 | 1.414 | 0.025 | | | | | | | | | |
| ZNF618 | NM_133374 | 1.417 | 0.009 | | | | | | | | | |
| ATF3 | NM_001040619 | 1.418 | 0.039 | Drexhage, 2010 - | | | | | | | | |
| | | | | PMID: 20633309 | | | | | | | | |
| MAPK8 | NM_002750 | 1.420 | 0.044 | | | | | | | | | |
| ATOH8 | NM_032827 | 1.423 | 0.014 | | | | | | | | | |
| CA11 | NM_001217 | 1.424 | 0.011 | | | | | | | | | |
| MYT1 | NM_004535 | 1.425 | 0.036 | Jungerius, 2007 - | | | | | | | | |
| | | | | PMID: 17893707 | | | | | | | | |
| C2CD2 | NM_015500 | 1.427 | 0.002 | | | | | | | | | |
| SALL4 | NM_020436 | 1.427 | 0.015 | | | | | | | | | |
| TTC6 | BC103915 | 1.428 | 0.037 | | | | | | | | | |
| SLC41A2 | NM_032148 | 1.428 | 0.040 | | | | | | | | | |
| QPCT | NM_012413 | 1.432 | 0.020 | | | | | | | | | |
| TRIB1 | NM_025195 | 1.433 | 0.010 | | | | | | | | | |
| TRPS1 | NM_014112 | 1.434 | 0.005 | | | | | | | | | |
| B3GALT1 | NM_020981 | 1.436 | 0.037 | | | | | | | | | |
| TIMP2 | NM_003255 | 1.438 | 0.018 | | | | | | | | | |
| ROBO3 | NM_022370 | 1.441 | 0.030 | | | | | | | | | |
| FAM107B | BC072452 | 1.442 | 0.005 | | | | | X | | | | |
| HFM1 | NM_001017975 | 1.444 | 0.036 | | | | | | | | | |
| STOX2 | NM_020225 | 1.446 | 0.004 | | | | | | | | | |
| ZNF626 | NM_145297 | 1.461 | 0.009 | | | | | | | | | |
| LGALS8 | NM_006499 | 1.462 | 0.039 | | | | | X | | | | |
| ZNF841 | NM_001136499 | 1.463 | 0.003 | | | | | | | | | |
| DUSP5P | NR_002834 | 1.463 | 0.026 | | | | | | | | | |
| HSPA1B | NM_005346 | 1.464 | 0.006 | Kim, 2008 - | | | | | | | | |
| | | | | PMID: 18299791 | | | | | | | | |
| HSPA1B | NM_005346 | 1.464 | 0.005 | Kim, 2008 - | | | | | | | | |
| | | | | PMID: 18299792 | | | | | | | | |
| HSPA1B | NM_005346 | 1.464 | 0.005 | Kim, 2008 - | | | | | | | | |
| | | | | PMID: 18299793 | | | | | | | | |
| ZYG11A | NM_001004339 | 1.468 | 0.023 | | | | | | | | | |
| ZIC2 | NM_007129 | 1.474 | 0.018 | Fallin, 2005 - | | | | | | | | |
| | | | | PMID: 16380905 | | | | | | | | |
| PTGDS | NM_000954 | 1.475 | 0.026 | Li, 2008 - PMID: | | | | | | | | |
| | | | | 18349703 | | | | | | | X | |
| PDE4DIP | NM_022359 | 1.475 | 0.032 | | | | | X | | X | | |
| IFI44 | NM_006417 | 1.480 | 0.023 | | | | | | | | | |
| RNASEL | NM_021133 | 1.482 | 0.017 | | | | | | | | | |
| HECTD2 | NM_182765 | 1.486 | 0.023 | | | | | | | | | |
| ZNF295 | NM_001098402 | 1.491 | 0.015 | | | | | | | | | |
| C1QTNF6 | NM_031910 | 1.496 | 0.002 | Takahashi, 2003 - | | | | | | | | |
| | | | | PMID: 12815732 | | | | | | | | |
| XRRA1 | NM_182969 | 1.498 | 0.001 | | | | | | | | | |
| CACNA2D2 | NM_001005505 | 1.501 | 0.049 | | | | | | | | X | |
| ADAMTS18 | NM_199355 | 1.507 | 0.008 | | | | | | | | | |
| JAZF1 | NM_175061 | 1.514 | 0.033 | | | | | | | | | |
| EPB41 | NM_203342 | 1.517 | 0.006 | | | | | | X | | | |
| CMIP | NM_198390 | 1.519 | 0.006 | | | | | | | | | |
| MRAS | NM_012219 | 1.520 | 0.005 | | | | | | | | | |
| S1PR3 | NM_005226 | 1.521 | 0.001 | | | | | | | | | |
| SAMD9 | NM_017654 | 1.526 | 0.036 | | | | | | | | | |
| MYL12A | NM_006471 | 1.528 | 0.009 | | | | | | | | | |
| FGD6 | NM_018351 | 1.532 | 0.010 | | | | | | | | | |
| ZNF536 | NM_014717 | 1.533 | 0.006 | | | | | | | | | |
| MSX2 | NM_002449 | 1.534 | 0.028 | | | | | | | | | |
| HSPA1A | NM_005345 | 1.534 | 0.018 | Kim, 2008 - | | | | | | | | |
| | | | | PMID: 18299791 | | | | | | | | |
| DISP1 | NM_032890 | 1.540 | 0.033 | | | | | | | | | |
| HSPA1A | NM_005345 | 1.540 | 0.018 | Kim, 2008 - | | | | | | | | |
| | | | | PMID: 18299792 | | | | | | | | |
| BMI1 | NM_005180 | 1.542 | 0.024 | | | | | | | | X | |
| WT1 | NM_024424 | 1.542 | 0.010 | | | | | | | | | |
| NOX4 | NR_026571 | 1.543 | 0.013 | | | | | | | | | |
| LMNA | NM_170707 | 1.557 | 0.031 | | | | | | X | | | |
| SPOCK3 | NM_001040159 | 1.567 | 0.046 | | | X | | | X | | | |
| VASH2 | NM_024749 | 1.567 | 0.017 | | | | | X | | | | |
| MAP3K13 | NM_004721 | 1.570 | 0.029 | | | | | X | | | | |
| TOX3 | NM_001080430 | 1.574 | 0.030 | | | | | | | | | |
| C10orf118 | NM_018017 | 1.574 | 0.002 | | | | | | | | | |
| EDIL3 | NM_005711 | 1.575 | 0.033 | | X | | | | | | X | |
| CPS1 | NM_001875 | 1.577 | 0.049 | | | | | | | | | |
| DCT | NM_001922 | 1.579 | 0.009 | | | | | | | | | |
| ZNF737 | NM_001159293 | 1.581 | 0.024 | | | | | | | | | |
| DEPDC6 | NM_022783 | 1.588 | 0.034 | | | | | | | | | |
| CDC42EP3 | NM_006449 | 1.595 | 0.003 | | | | | | X | | X | |
| SIPA1L2 | NM_020808 | 1.599 | 0.003 | | | | | | | | | |
| GLRA1 | NM_001146040 | 1.603 | 0.031 | | | | | | | | | |
| PRSS12 | NM_003619 | 1.605 | 0.026 | | | | | | | | | |
| BSCL2 | NM_001130702 | 1.606 | 0.044 | | | | | | | | | |
| FGFR1 | NM_023110 | 1.608 | 0.001 | Jungerius, 2007 - | | | | | | | | |
| | | | | PMID: 17893707 | | | | | | | | |
| PARP14 | NM_017554 | 1.619 | 0.050 | | | | | | | | | |
| RHOBTB3 | NM_014899 | 1.625 | 0.032 | | | | X | | X | X | | |
| DUSP5P | AK055963 | 1.632 | 0.017 | | | | | | | | | |
| LRRC55 | NM_001005210 | 1.642 | 0.034 | | | | | | | | | |
| VEGFC | NM_005429 | 1.647 | 0.022 | | | | | | | | | |
| KIAA1199 | NM_018689 | 1.653 | 0.026 | | | | | | | | | |
| SFRP2 | NM_003013 | 1.657 | 0.042 | | | | | | | | | |
| FAM190A | NM_001145065 | 1.668 | 0.048 | | | | | | | | | |
| CCNG1 | NM_004060 | 1.685 | 0.001 | | | | | | | | | |
| NRG1 | NM_013958 | 1.691 | 0.004 | Addington, 2006 - | | | | | | | | |
| | | | | PMID: 17033632 | | | | | | | | |
| ENPP2 | NM_006209 | 1.695 | 0.041 | | | X | | | | | X | |
| RARB | NM_000965 | 1.697 | 0.007 | | | | | | | | | |
| ATL3 | ENST00000398868 | 1.697 | 0.029 | | | | | | | | | |
| CAMK1D | NM_153498 | 1.699 | 0.012 | | | | | | | | | |
| KRTAP5-2 | NM_001004325 | 1.702 | 0.026 | | | | | | | | | |
| RBM24 | NM_001143942 | 1.704 | 0.027 | Lin, 2009 - PMID: | | | | | | | | |
| | | | | 19694819 | | | | | | | | |
| ZFHX3 | NM_006885 | 1.705 | 0.004 | | | | | | | | | |
| IGFBPL1 | NM_001007563 | 1.708 | 0.009 | | | | | | | | | |
| LAMA4 | NM_001105206 | 1.716 | 0.023 | | | | | | | | | |
| ANKRD44 | NM_153697 | 1.717 | 0.018 | | | | | | | | | |
| MAL | NM_002371 | 1.720 | 0.048 | Jungerius, 2007 - | | | | | | | | |
| | | | | PMID: 17893707 | | | | | | | | |
| PCDHB2 | NM_018936 | 1.721 | 0.037 | | | | | | | | | |
| MYOF | NM_013451 | 1.736 | 0.023 | | | | | | | | | |
| SLAIN1 | NM_001040153 | 1.738 | 0.037 | | | | | | | | | |
| RBM9 | NM_001082578 | 1.745 | 0.039 | Amagane, 2010 - | | X | | | | | X | |
| | | | | PMID: 20188514 | | | | | | | | |
| NR4A2 | NM_006186 | 1.748 | 0.028 | Buervenich, 2000 - | X | | X | X | | X | | |
| | | | | PMID: 11121187 | | | | | | | | |
| RLBP1L1 | NM_173519 | 1.749 | 0.045 | | | | | | | | | |
| DUSP4 | NM_001394 | 1.750 | 0.039 | | | | | | | | | |
| CA14 | NM_012113 | 1.755 | 0.002 | | | | | | | | | |
| ATL3 | NM_015459 | 1.761 | 0.028 | | | | | | | | | |
| RIMS2 | NM_001100117 | 1.764 | 0.031 | Weidenhofer, | | | | | | | | |
| | | | | 2009 - PMID: | | | | | | | | |
| | | | | 18490030 | | | | | | | | |
| GCNT4 | NM_016591 | 1.765 | 0.011 | | | | | | | | | |
| GDF10 | NM_004962 | 1.774 | 0.012 | | | | | | | | | |
| PLEKHG4B | NM_052909 | 1.777 | 0.020 | | | | | | | | | |
| LINGO2 | NM_152570 | 1.779 | 0.018 | | | | | | | | | |
| CYFIP2 | NM_001037332 | 1.790 | 0.003 | | | | | | | | | |
| DDX60L | NM_001012967 | 1.796 | 0.032 | | | | | | | | | |
| ADAMTSL1 | NM_001040272 | 1.801 | 0.043 | | | | | | | | | |
| PHLDA1 | NM_007350 | 1.827 | 0.027 | | | | | X | | | | |
| NQO1 | NM_000903 | 1.833 | 0.047 | Hori, 2003 - | | | | | | | | |
| | | | | PMID: 12834817 | | | | | | | | |
| PDE4D | NM_001104631 | 1.843 | 0.000 | Tomppo, 2009 - | | | | | | | | |
| | | | | PMID: 19251251 | | | | | | | | |
| LNX1 | NM_001126328 | 1.844 | 0.017 | | | | | | | | | |
| RTL1 | NM_001134888 | 1.849 | 0.027 | | | | | | | | | |
| GRB10 | NM_001001555 | 1.852 | 0.005 | | | | | | | | | |
| ZNF423 | NM_015069 | 1.881 | 0.012 | | | | | | | | | |
| RND3 | NM_005168 | 1.881 | 0.024 | | | | | | | | | |
| C11orf41 | NM_012194 | 1.885 | 0.045 | | | | | | | | | |
| NALCN | NM_052867 | 1.894 | 0.031 | Souza, 2010c - | | | | | | | | |
| | | | | PMID: 20674038 | | | | | | | | |
| SH3BP5 | NM_004844 | 1.897 | 0.015 | | | | | | | | | |
| STXBP5 | NM_001127715 | 1.900 | 0.037 | | | | | | | | | |
| STMN4 | NM_030795 | 1.902 | 0.021 | | | | | | | | | |
| IFITM3 | NM_021034 | 1.906 | 0.018 | | | | | | X | | | X |
| PPARGC1A | NM_013261 | 1.935 | 0.030 | Christoforou, 2007 - | | X | | | | | | |
| | | | | PMID: 17457313 | | | | | | | | |
| COL4A2 | NM_001846 | 1.950 | 0.022 | | | | | | X | | | |
| FLJ41170 | AK123165 | 1.972 | 0.033 | | | | | | | | | |
| BZW2 | NM_001159767 | 1.987 | 0.031 | | | | | | | | | |
| GLI3 | NM_000168 | 1.994 | 0.001 | | | | | | | | | |
| NKAIN2 | NM_001040214 | 1.996 | 0.014 | | | | | | | | | |
| TLE1 | NM_005077 | 2.000 | 0.007 | | | | | | | | | |
| SETBP1 | NM_015559 | 2.000 | 0.010 | | | | | | | | | |
| NET1 | NM_001047160 | 2.013 | 0.001 | | | | | | | | | |
| TNFRSF11B | NM_002546 | 2.016 | 0.011 | | | | | | | | | |
| PIP5K1B | NM_003558 | 2.032 | 0.025 | Ikeda, 2009 - | | | | | | | | |
| | | | | PMID: 19850283 | | | | | | | | |
| ZNF154 | NM_001085384 | 2.036 | 0.005 | | | | | | | | | |
| CHMP1B | NM_020412 | 2.070 | 0.012 | | | | | | | | | |
| LEF1 | NM_016269 | 2.071 | 0.050 | | | | | | | | | |
| GFPT2 | NM_005110 | 2.097 | 0.010 | | | | | | | | X | |
| ARHGAP18 | NM_033515 | 2.106 | 0.002 | Potkin, 2009 - | | | | | | | | |
| | | | | PMID: 19065146 | | | | | | | | |
| COL4A1 | NM_001845 | 2.137 | 0.038 | | | | | | | | | |
| DUSP1 | NM_004417 | 2.145 | 0.029 | | X | | | | | | | |
| HECW1 | NM_015052 | 2.148 | 0.016 | | | | | | | | | |
| ANGPT1 | NM_001146 | 2.149 | 0.005 | | | | | | | | | |
| IFITM2 | NM_006435 | 2.154 | 0.048 | | | | | | | | | |
| NRP2 | NM_201266 | 2.157 | 0.008 | | | | | | | | | |
| LRRTM4 | NM_024993 | 2.197 | 0.041 | | | | | | | | | |
| ZIC4 | NM_032153 | 2.238 | 0.005 | | | | | | | | | |
| MST131 | ENST00000423322 | 2.269 | 0.012 | | | | | | | | | |
| NT5E | NM_002526 | 2.276 | 0.015 | | | | | | | | | |
| CFI | NM_000204 | 2.291 | 0.035 | | | | | | | | | |
| GNG2 | NM_053064 | 2.330 | 0.046 | | | | | | | | | |
| SLIT2 | NM_004787 | 2.342 | 0.019 | | | | | X | | | | |
| PLXNA2 | NM_025179 | 2.376 | 0.014 | Betcheva, 2009 - | | | | | X | | | |
| | | | | PMID: 19158809 | | | | | | | | |
| EPAS1 | NM_001430 | 2.379 | 0.038 | | | | | X | | | | |
| SLC16A9 | NM_194298 | 2.379 | 0.006 | | | | | | | | | |
| TOX | NM_014729 | 2.424 | 0.007 | | | | | | | | X | |
| SV2C | NM_014979 | 2.437 | 0.014 | | X | | | | | | | |
| C6orf142 | NM_138569 | 2.448 | 0.035 | | | | | | | | | |
| EBF1 | NM_024007 | 2.455 | 0.008 | | | | | | | | | |
| MAMDC2 | NM_153267 | 2.489 | 0.035 | | | | | | | | | |
| CYP26A1 | NM_000783 | 2.498 | 0.000 | | | | | | | | | |
| NEFL | NM_006158 | 2.508 | 0.033 | Fallin, 2005 - | | | | | X | | | |
| | | | | PMID: 16380905 | | | | | | | | |
| LOC151760 | ENST00000383686 | 2.513 | 0.014 | | | | | | | | | |
| ANK3 | NM_020987 | 2.522 | 0.026 | Athanasiu, 2010 - | | X | | | | | | |
| | | | | PMID: 20185149 | | | | | | | | |
| FGF14 | NM_175929 | 2.527 | 0.038 | Jungerius, 2007 - | | | | | | | | |
| | | | | PMID: 17893707 | | | | | | | | |
| PXDNL | NM_144651 | 2.554 | 0.031 | | | | | | | | | |
| CACNA2D3 | NM_018398 | 2.558 | 0.003 | | | | | | | | | |
| UNC5C | NM_003728 | 2.564 | 0.003 | Ikeda, 2009 - | | | | | | | | |
| | | | | PMID: 19850283 | | | | | | | | |
| EFNA5 | NM_001962 | 2.595 | 0.004 | | | | | | | | | |
| FRAS1 | NM_025074 | 2.623 | 0.015 | | | | | | | | | |
| DACH1 | NM_080759 | 2.674 | 0.026 | | | | | | | | | |
| GNAL | NM_182978 | 2.675 | 0.046 | Schwab, 1998 - | | | | | | | | |
| | | | | PMID: 9758604 | | | | | | | | |
| ECEL1 | NM_004826 | 2.877 | 0.016 | | | | | | | | | |
| ARHGAP29 | NM_004815 | 2.893 | 0.002 | | | | | | | | | |
| COL12A1 | NM_004370 | 2.916 | 0.035 | | | | | | | | | |
| MATN2 | NM_002380 | 2.920 | 0.001 | | | | | | | | | |
| EBF3 | NM_001005463 | 2.936 | 0.012 | O'Donovan, 2008 - | | | | | | | | |
| | | | | PMID: 18677311 | | | | | | | | |
| PDE1C | NM_005020 | 2.963 | 0.041 | | | | | | | | | |
| HS3ST3B1 | NM_006041 | 3.091 | 0.022 | | | | | | | | | |
| ALPK2 | NM_052947 | 3.124 | 0.029 | | | | | | | | | |
| THSD7A | NM_015204 | 3.142 | 0.020 | | | | | | | | | |
| PDE7B | NM_018945 | 3.155 | 0.011 | Amann- | | | | | | | | |
| | | | | Zalcenstein, 2006 - | | | | | | | | |
| | | | | PMID: 16773125 | | | | | | | | |
| PAPPA | NM_002581 | 3.165 | 0.016 | | | | | | | | | |
| FAM46A | NM_017633 | 3.188 | 0.004 | | | | | | | | | |
| FIGN | NM_018086 | 3.202 | 0.020 | | | | | | | | | |
| DCC | NM_005215 | 3.302 | 0.027 | Speight, 2000 - | | | | | | | | |
| | | | | PMID: 10889538 | | | | | | | | |
| CNTN3 | NM_020872 | 3.355 | 0.016 | | | | | | | | | |
| CTSC | NM_001814 | 3.375 | 0.017 | | | | | | | | | |
| SLFN5 | NM_144975 | 3.405 | 0.034 | | | | | | | | | |
| ATP8A1 | NM_006095 | 3.528 | 0.043 | | | | | | | | | |
| MAB21L1 | NM_005584 | 3.567 | 0.049 | | | | | | | | | |
| NEFM | NM_005382 | 3.676 | 0.020 | Fallin, 2005 - | | | | | | | | |
| | | | | PMID: 16380905 | | | | | | | | |
| RUNX1T1 | NM_175634 | 3.906 | 0.012 | | | | | | | | | |
| ASS1 | NM_000050 | 3.975 | 0.011 | | | | | | | | | |
| FAT4 | NM_024582 | 4.384 | 0.005 | | | | | | | | | |
| ZMAT4 | NM_024645 | 4.434 | 0.009 | | | | | | | | | |
| VGLL3 | NM_016206 | 4.557 | 0.003 | | | | | | | | | |
| IFITM1 | NM_003641 | 4.844 | 0.028 | | | | | | | | | |
| SNORD113-3 | NR_003231 | 5.036 | 0.043 | | | | | | | | | |
| SNORD114-3 | NR_003195 | 5.156 | 0.047 | | | | | | | | | |
| SERPINI1 | NM_001122752 | 5.615 | 0.005 | | | | | | | | | |
| PAX3 | NM_181458 | 6.139 | 0.006 | Jungerius, 2007 - | | | | | | | | |
| | | | | PMID: 17893707 | | | | | | | | |

TABLE 6
GO analysis of microarray data identified significantly affected
pathways in SCZD hiPSC neurons.

| | Network | Statistically Significant GO | Genes Significantly Up, | | Genes Significantly Down, | |
|---|---|---|---|---|---|---|
| p-value | Objects | Pathways | Fold Change | | Fold Change | |
| 2.93E−06 | 21/111 | Cytoskeleton Remodeling via | WNT2B, | 1.41 | WNT7A, | −1.62 |
| | | TGF and Wnt | WNT3, | 1.31 | ACTN2, | −2.33 |
| | | | AXIN2, | 1.39 | | |
| | | | LEF1, | 2.07 | | |
| | | | TCF4, | 1.33 | | |
| | | | PIK3R3, | 1.4 | | |
| | | | COL4A1, | 2.14 | | |
| | | | COL4A2, | 1.95 | | |
| 6.39E−06 | 13/50 | Function of MEF2 | | | ITPR2, | −1.72 |
| | | | | | PRKCA, | −2.41 |
| | | | | | KAT2B, | −1.56 |
| 1.10E−05 | 12/45 | α6-β4-integrins | NRG1, | 1.69 | EGF, | −1.98 |
| | | | PDPK1, | 1.26 | ERBB3, | −1.21 |
| | | | | | MST1, | −1.41 |
| | | | | | PRKCA, | −2.41 |
| | | | | | ITPR2, | −1.72 |
| 2.34E−05 | 10/34 | Erk signal transduction | GRIN1, | 1.16 | GRIN2A, | −1.72 |
| | | | PTPRR, | 1.27 | PTPRE, | −1.7 |
| | | | | | PRKCA, | −2.41 |
| | | | | | CAMK1, | −1.12 |
| 3.91E−05 | 11/43 | Notch signaling pathway | HDAC2, | 1.29 | PSENEN, | −1.31 |
| | | | | | NOTCH1, | −1.9 |
| | | | | | DLL1, | −1.92 |
| | | | | | HEY2, | −4.41 |
| | | | | | KAT2B, | −1.56 |
| | | | | | KAT2A, | −1.38 |
| 6.48E−05 | 12/53 | Wnt signaling pathway | WNT2B, | 1.41 | WNT7A, | −1.62 |
| | | | WNT3, | 1.31 | LRP5, | −1.29 |
| | | | WNT7B, | 1.14 | | |
| | | | AXIN, | 1.39, | | |
| | | | LEF1, | 2.07 | | |
| | | | TCF4, | 1.33 | | |
| 6.76E−05 | 10/38 | cAMP signaling pathway | GNG2, | 2.33 | ADCY7, | −1.31 |
| | | | PRKAR2A, | 1.25 | ADCY8, | −2.03 |
| | | | PPP3CB, | 1.23 | PRKCA, | −2.41 |
| 8.52E−05 | 15/80 | NMDA-dependent | GRIN1, | 1.16 | GRIN2A, | −1.72 |
| | | postsynaptic long-term | GRM1, | 1.27 | ADCY8, | −2.03 |
| | | potentiation | PRKAR2A, | 1.25 | PRKCA, | −2.41 |

TABLE 7
Analysis of microarray data of genes affected by CNVs identified in SCZD patients.

| | | | | | Fold- | | |
|---|---|---|---|---|---|---|---|
| | | | | | Change | | Predicted |
| | | | | | (Affected | | By |
| Affected | Type of | | | CNV | Patient vs | Expression | Genotype |
| Patient | CNV | CNV location | Gene Affected | p-value | Control) | p-value | (p < 0.05) |
| Patient 1 | deletion | chr2: 78551233-78576088 | BC024248 | 3.3E−11 | −1.04 | 0.1062 | |
| Patient 1 | duplication | chr5: 17669005-17706691 | none | 4.4E−09 | na | na | |
| Patient 1 | deletion | chr7: 110838909-110985533 | IMMP2L | 7.2E−48 | −1.11 | 0.0360 | X |
| Patient 1 | deletion | chr8: 3607327-3648499 | CSMD1 | 1.0E−46 | −1.31 | 0.0010 | X |
| Patient 1 | duplication | chr8: 36372115-36392410 | none | 5.8E−13 | na | na | |
| Patient 1 | duplication | chr10: 44529265-44679123 | AK056518 | 0.0E+00 | nd | nd | |
| Patient 1 | duplication | chr12: 89860232-89930609 | EPYC | 0.0E+00 | −1.04 | 0.1767 | |
| Patient 1 | duplication | chr12: 89860232-89930609 | C12Orf12 | 0.0E+00 | −1.06 | 0.1217 | |
| Patient 1 | deletion | chr18: 40170762-40333519 | BC051727 | 5.1E−110 | nd | nd | |
| Patient 2 | deletion | chr2: 17083353-17103531 | none | 3.1E−11 | na | na | |
| Patient 2 | duplication | chr3: 156964346-156985822 | C3orf33 | 1.7E−07 | −1.06 | 0.1658 | |
| Patient 2 | deletion | chr4: 8408598-8424007 | ACOX3 | 1.1E−20 | −1.02 | 0.0509 | |
| Patient 2 | deletion | chr4: 8408598-8424007 | AX746755 | 1.1E−20 | nd | nd | |
| Patient 2 | deletion | chr5: 27642965-27665975 | none | 2.2E−15 | na | na | |
| Patient 2 | duplication | chr6: 74514699-74534191 | CD109 | 1.1E−16 | −1.00 | 0.9222 | |
| Patient 2 | deletion | chr8: 2126589-2147674 | AY156957 | 1.0E−10 | nd | nd | |
| Patient 2 | deletion | chr8: 2126589-2147674 | AX747124 | 1.0E−10 | nd | nd | |
| Patient 2 | deletion | chr8: 3969433-4064034 | CSMD1 (Intron) | 1.0E−36 | −1.04 | 0.5377 | |
| Patient 2 | deletion | chr8: 18896564-18912764 | PSD3 (Intron) | 1.7E−07 | −1.00 | 0.9401 | |
| Patient 2 | duplication | chr16: 55217605-55276937 | MT1E | 7.7E−14 | −1.03 | 0.4904 | |
| Patient 2 | duplication | chr16: 55217605-55276937 | MT1M | 7.7E−14 | −1.01 | 0.7144 | |
| Patient 2 | duplication | chr16: 55217605-55276937 | MTB | 7.7E−14 | nd | nd | |
| Patient 2 | duplication | chr16: 55217605-55276937 | MT1A | 7.7E−14 | −1.04 | 0.1029 | |
| Patient 2 | duplication | chr16: 55217605-55276937 | MT1B | 7.7E−14 | −1.02 | 0.1023 | |
| Patient 2 | duplication | chr16: 55217605-55276937 | MTM | 7.7E−14 | nd | nd | |
| Patient 2 | duplication | chr16: 55217605-55276937 | MT1F | 7.7E−14 | −1.08 | 0.0198 | |
| Patient 2 | duplication | chr16: 55217605-55276937 | MT1G | 7.7E−14 | −1.03 | 0.3647 | |
| Patient 2 | duplication | chr16: 55217605-55276937 | MT1H | 7.7E−14 | −1.04 | 0.2343 | |
| Patient 2 | duplication | chr16: 55217605-55276937 | MT1IP | 7.7E−14 | −1.02 | 0.2244 | |
| Patient 2 | duplication | chr16: 55217605-55276937 | MTW | 7.7E−14 | nd | nd | |
| Patient 2 | duplication | chr16: 55217605-55276937 | MT1X | 7.7E−14 | −1.08 | 0.0407 | |
| Patient 2 | duplication | chr19: 34010221-34052245 | none | 0.0E+00 | na | na | |
| Patient 3 | duplication | chr1: 119726536-119739044 | HAO2 | 2.2E−16 | 1.01 | 0.7481 | |
| Patient 3 | deletion | chr2: 17083353-17103531 | none | 5.7E−12 | na | na | |
| Patient 3 | deletion | chr4: 8408598-8422722 | ACOX3 | 6.5E−27 | −1.03 | 0.0112 | X |
| Patient 3 | deletion | chr4: 8408598-8422722 | AX746755 | 6.5E−27 | nd | nd | |
| Patient 3 | duplication | chr6: 74514699-74532358 | CD109 | 9.7E−10 | −1.04 | 0.4356 | |
| Patient 3 | deletion | chr8: 2126589-2147674 | AY156957 | 9.2E−12 | nd | nd | |
| Patient 3 | deletion | chr8: 2126589-2147674 | AX747124 | 9.2E−12 | nd | nd | |
| Patient 3 | deletion | chr8: 18896564-18909227 | PSD3 (Intron) | 3.0E−08 | 1.07 | 0.0006 | |
| Patient 3 | deletion | chr18: 1894094-1974770 | none | 1.9E−14 | na | na | |
| Patient 3 | duplication | chr19: 50838214-50889101 | EML2 | 1.1E−16 | −1.02 | 0.3370 | |
| Patient 3 | duplication | chr19: 50838214-50889101 | GIPR | 1.1E−16 | 1.05 | 0.0016 | X |
| Patient 3 | duplication | chr19: 50838214-50889101 | SNRPD2 | 1.1E−16 | 1.07 | 0.0001 | X |
| Patient 4 | duplication | chr3: 199196414-199382747 | LMLN | 3.1E−13 | −1.03 | 0.0161 | |
| Patient 4 | duplication | chr3: 199196414-199382747 | LOC348840 | 3.1E−13 | −1.01 | 0.8604 | |
| Patient 4 | duplication | chr5: 160951572-160965569 | GABRB2 | 1.4E−12 | −1.04 | 0.0000 | |
| | | | (5′ Intergenic) | | | | |
| Patient 4 | duplication | chr5: 160951572-160965569 | GABRA6 | 1.4E−12 | 1.01 | 0.5703 | |
| | | | (5′ Intergenic) | | | | |
| Patient 4 | duplication | chr6: 162538658-162595733 | PARK2 | 0.0E+00 | 1.01 | 0.6628 | |
| Patient 4 | deletion | chr7: 151346486-151441286 | GALNT5 | 1.0E−59 | 1.69 | 0.0000 | |
| Patient 4 | deletion | chr7: 151346486-151441286 | GALNT11 | 1.0E−59 | −1.10 | 0.0000 | X |
| Patient 4 | deletion | chr8: 15464189-15485502 | TUSC3 (Intron) | 8.3E−25 | −1.01 | 0.3024 | |
| Patient 4 | deletion | chr10: 84525405-84556365 | NRG3 isoform 2 | 6.9E−24 | −1.22 | 0.0005 | X |
| Patient 4 | deletion | chr10: 96489466-96533096 | CYP2C19 | 4.4E−18 | 1.04 | 0.1978 | |
| Patient 4 | deletion | chr11: 18556183-18577561 | UEVLD | 2.2E−20 | −1.13 | 0.0018 | X |
| Patient 4 | deletion | chr12: 8211354-8666816 | ZNF705A | 2.1E−40 | 1.03 | 0.4175 | |
| Patient 4 | deletion | chr12: 8211354-8666816 | FAM90A1 | 2.1E−40 | −1.03 | 0.0540 | |
| Patient 4 | deletion | chr12: 8211354-8666816 | CLEC6A | 2.1E−40 | 1.06 | 0.1207 | |
| Patient 4 | deletion | chr12: 8211354-8666816 | CLEC4D | 2.1E−40 | −1.02 | 0.7962 | |
| Patient 4 | deletion | chr12: 8211354-8666816 | CLEC4E | 2.1E−40 | −1.04 | 0.5424 | |
| Patient 4 | deletion | chr12: 8211354-8666816 | AICDA | 2.1E−40 | 1.03 | 0.0545 | |
| Patient 4 | deletion | chr12: 8211354-8666816 | CR611653 | 2.1E−40 | nd | nd | |
| Patient 4 | duplication | chr12: 50517243-50577958 | ANKRD33 | 0.0E+00 | 1.02 | 0.5990 | |
| Patient 4 | duplication | chr12: 69784529-69797993 | TSPAN8 | 2.9E−12 | 1.15 | 0.1061 | |
| | | | (3′ Intergenic) | | | | |
| Patient 4 | duplication | chr15: 50782920-50827258 | KIAA1370 | 0.0E+00 | −1.06 | 0.0125 | |
| | | | (5′ Intergenic) | | | | |
| Patient 4 | duplication | chr15: 50782920-50827258 | ONECUT1 | 0.0E+00 | 1.00 | 0.8738 | |
| | | | (3′ Intergenic) | | | | |
| Patient 4 | duplication | chr16: 9803856-9814779 | GRIN2A | 9.9E−07 | −1.16 | 0.0000 | |
| | | | (Intron)* | | | | |
| Patient 4 | duplication | chr17: 9923061-10356441 | GAS7 | 0.0E+00 | 1.08 | 0.0137 | X |
| Patient 4 | duplication | chr17: 9923061-10356441 | MYH13 | 0.0E+00 | 1.02 | 0.0825 | |
| Patient 4 | duplication | chr17: 9923061-10356441 | MYH8 | 0.0E+00 | 1.02 | 0.5058 | |
| Patient 4 | duplication | chr17: 9923061-10356441 | MYH4 | 0.0E+00 | 1.33 | 0.0065 | X |
| Patient 4 | duplication | chr17: 9923061-10356441 | MYH1 | 0.0E+00 | 1.89 | 0.0000 | X |
| Patient 4 | deletion | chr18: 156081-166915 | USP14* | 8.3E−07 | 1.03 | 0.0299 | |
| Patient 4 | deletion | chr20: 61385006-61408612 | ARFGAP1 | 6.1E−09 | −1.02 | 0.0047 | X |
| Patient 4 | deletion | chr20: 61385006-61408612 | COL20A1 | 6.1E−09 | 1.16 | 0.0001 | |
| Patient 4 | deletion | chr20: 61385006-61408612 | KIAA1510 | 6.1E−09 | nd | nd | |

TABLE 8
Expression analysis of top Affymetrix transcripts misexpressed at
well-characterized SCZD CNVs

| | | | | Fold-Change |
|---|---|---|---|---|
| Transcript | | | p-value | (SCZD vs |
| ID | Cytoband | Gene Symbol | (Diagnosis) | Control) |
| 7919168 | 1q21.1 | PDE4DIP | 0.03176 | 1.48 |
| 7919243 | 1q21.1 | CD160 | 0.06112 | 1.10 |
| 7904907 | 1q21.1 | BCL9 | 0.06162 | 1.16 |
| 7919226 | 1q21.1 | POLR3C | 0.06511 | 1.17 |
| 7919195 | 1q21.1 | | 0.06621 | −1.08 |
| 7904883 | 1q21.1 | CHD1L | 0.06630 | −1.22 |
| 7904480 | 1q21.1 | | 0.06819 | −1.24 |
| 7904963 | 1q21.1 | | 0.06819 | −1.24 |
| 7919193 | 1q21.1 | NUDT4P1 | 0.02777 | −1.27 |
| 7919299 | 1q21.1 | LOC100130236 | 0.09582 | −1.10 |
| 7981775 | 15q11.2 | DKFZP547L112 | 0.01185 | −1.19 |
| 7981781 | 15q11.2 | OR4M2 | 0.03283 | −1.23 |
| 7986685 | 15q11.2 | DEXI | 0.04303 | −1.12 |
| 7986601 | 15q11.2 | LOC440243 | 0.04308 | 1.12 |
| 7981773 | 15q11.2 | | 0.05008 | −1.15 |
| 7981752 | 15q11.2 | GOLGA8D | 0.11046 | −1.11 |
| 7981785 | 15q11.2 | OR4N3P | 0.11468 | 1.11 |
| 7986563 | 15q11.2 | LOC646057 | 0.11968 | −1.14 |
| 7981824 | 15q11.2 | CYFIP1 | 0.13054 | −1.14 |
| 7986603 | 15q11.2 | LOC646214 | 0.16966 | 1.09 |
| 7986820 | 15q13.1 | | 0.09880 | −1.07 |
| 7982102 | 15q13.1 | GABRA5 | 0.15919 | 1.18 |
| 7982127 | 15q13.1 | | 0.53415 | −1.03 |
| 7986789 | 15q13.1 | ATP10A | 0.53850 | 1.09 |
| 7986822 | 15q13.1 | GABRB3 | 0.70732 | −1.08 |
| 7982117 | 15q13.1 | GABRG3 | 0.84759 | −1.07 |
| 7982100 | 15q13.1 | | 0.91167 | −1.01 |
| 7986836 | 15q13.1 | | 0.93823 | 1.00 |
| 7982131 | 15q13.2 | GOLGA8G | 0.07137 | −1.12 |
| 7986922 | 15q13.2 | GOLGA8G | 0.07137 | −1.12 |
| 7991695 | 15q13.2 | GOLGA8D | 0.14069 | −1.11 |
| 7986947 | 15q13.2 | GOLGA9P | 0.28404 | −1.13 |
| 7986863 | 15q13.2 | HERC2 | 0.41947 | 1.05 |
| 7982129 | 15q13.2 | RPL41 | 0.50117 | 1.07 |
| 7986945 | 15q13.2 | | 0.61305 | −1.06 |
| 7982152 | 15q13.2 | | 0.62512 | −1.03 |
| 7986943 | 15q13.2 | | 0.62512 | −1.03 |
| 7982154 | 15q13.2 | HERC2P2 | 0.78350 | 1.03 |
| 7986838 | 15q13.2 | OCA2 | 0.80749 | −1.05 |
| 7987048 | 15q13.3 | MTMR10 | 0.03792 | −1.27 |
| 7982299 | 15q13.3 | LOC390561 | 0.03795 | −1.28 |
| 7982185 | 15q13.3 | DEXI | 0.04303 | −1.12 |
| 7982252 | 15q13.3 | DKFZP434L187 | 0.12698 | 1.28 |
| 7982254 | 15q13.3 | | 0.16920 | 1.37 |
| 7982230 | 15q13.3 | GOLGA9P | 0.21265 | −1.14 |
| 7982271 | 15q13.3 | GOLGA9P | 0.21375 | −1.16 |
| 7987097 | 15q13.3 | | 0.24477 | −1.08 |
| 7986960 | 15q13.3 | FAM189A1 | 0.28169 | −1.10 |
| 7982204 | 15q13.3 | HMGN2 | 0.31280 | 1.07 |
| 7982290 | 15q13.3 | | 0.32056 | −1.24 |
| 7987012 | 15q13.3 | CHRFAM7A | 0.35454 | 1.13 |
| 7995320 | 16p11.1 | | 0.00256 | −1.13 |
| 7995348 | 16p11.1 | | 0.02650 | −1.12 |
| 7995338 | 16p11.1 | | 0.07405 | −1.06 |
| 7995322 | 16p11.1 | | 0.08523 | −1.11 |
| 8001111 | 16p11.1 | UBE2MP1 | 0.17026 | 1.16 |
| 7995336 | 16p11.1 | | 0.18118 | −1.11 |
| 7995324 | 16p11.1 | | 0.19448 | −1.10 |
| 7995330 | 16p11.1 | | 0.19978 | −1.14 |
| 7995334 | 16p11.1 | | 0.21771 | −1.06 |
| 7995326 | 16p11.1 | | 0.26439 | −1.09 |
| 7995206 | 16p11.2 | TGFB1I1 | 0.00371 | −1.28 |
| 7995007 | 16p11.2 | HSD3B7 | 0.02001 | −1.16 |
| 7995292 | 16p11.2 | SLC6A8 | 0.02141 | −1.10 |
| 7994541 | 16p11.2 | LAT | 0.02255 | −1.10 |
| 8000932 | 16p11.2 | C16orf93 | 0.03374 | −1.36 |
| 8000582 | 16p11.2 | SULT1A2 | 0.03606 | −1.25 |
| 7994371 | 16p11.2 | NPIPL3 | 0.03871 | −1.12 |
| 8000791 | 16p11.2 | YPEL3 | 0.04007 | −1.20 |
| 8000748 | 16p11.2 | HIRIP3 | 0.04325 | −1.12 |
| 8003583 | 16p11.2 | KIF22 | 0.04398 | −1.11 |
| 7994620 | 16p11.2 | KIF22 | 0.04517 | −1.11 |
| 8071206 | 22q11.21 | MRPL40 | 0.00560 | −1.21 |
| 8074194 | 22q11.21 | OR11H1 | 0.00893 | −1.06 |
| 8074591 | 22q11.21 | RIMBP3B | 0.01867 | −1.17 |
| 8071212 | 22q11.21 | CDC45L | 0.03503 | −1.20 |
| 8071368 | 22q11.21 | TMEM191A | 0.04611 | 1.15 |
| 8074204 | 22q11.21 | XKR3 | 0.04841 | 1.04 |
| 8074569 | 22q11.21 | GGT3P | 0.05390 | −1.08 |
| 8071063 | 22q11.21 | psiTPTE22 | 0.06227 | −1.16 |
| 8071259 | 22q11.21 | SEPT5 | 0.06754 | 1.24 |
| 8074316 | 22q11.21 | GGT3P | 0.07437 | −1.10 |
| 8074890 | 22q11.23 | | 0.00112 | −1.16 |
| 8071768 | 22q11.23 | SMARCB1 | 0.01106 | 1.08 |
| 8074769 | 22q11.23 | RIMBP3C | 0.02656 | −1.13 |
| 8074958 | 22q11.23 | | 0.09232 | 1.06 |
| 8071545 | 22q11.23 | | 0.10165 | −1.05 |
| 8071564 | 22q11.23 | | 0.11648 | −1.15 |
| 8074867 | 22q11.23 | POM121L1P | 0.12617 | 1.05 |
| 8071737 | 22q11.23 | MIF | 0.14101 | −1.26 |
| 8071676 | 22q11.23 | RAB36 | 0.14176 | −1.25 |
| 8074748 | 22q11.23 | PI4KAP2 | 0.14569 | −1.21 |
| 8074931 | 22q11.23 | ZNF70 | 0.16735 | 1.06 |

TABLE 9
Analysis of inheritance of CNVs identified in SCZD patient 4.

| Relation to | | | | CNV |
|---|---|---|---|---|
| Proband | Type of CNV | CNV location | | p-value |
| Father | deletion | chr2: | 6684377-6870214 | 3.87E−85 |
| Patient 5 | deletion | chr2: | 6683379-6870214 | 2.23E−119 |
| Patient 5 | deletion | chr3: | 128418717-128432973 | 8.82E−32 |
| Mother | duplication | chr3: | 199195203-199367408 | 7.86E−08 |
| Patient 4 | duplication | chr3: | 199196414-199376895 | 1.18E−11 |
| Father | duplication | chr6: | 162541018-162595733 | 3.59E−13 |
| Patient 4 | duplication | chr6: | 162541018-162595733 | 1.98E−13 |
| Patient 5 | duplication | chr6: | 162541018-162595733 | 2.22E−16 |
| Patient 5 | deletion | chr6: | 170195821-170206320 | 4.81E−12 |
| Father | deletion | chr7: | 111514132-111536768 | 1.66E−37 |
| Father | deletion | chr7: | 151348062-151439868 | 7.98E−39 |
| Patient 4 | deletion | chr7: | 151350927-151437530 | 7.98E−37 |
| Patient 5 | deletion | chr7: | 151350927-151439868 | 1.47E−55 |
| Father | deletion | chr8: | 18891576-18910636 | 7.79E−07 |
| Patient 4 | deletion | chr8: | 15456201-15484626 | 9.45E−10 |
| Mother | duplication | chr10: | 30514430-30526183 | 4.68E−10 |
| Patient 4 | deletion | chr10: | 84521261-84556365 | 5.26E−16 |
| Patient 5 | deletion | chr10: | 84522764-84556365 | 1.62E−28 |
| Patient 4 | deletion | chr10: | 96489466-96535919 | 3.39E−08 |
| Patient 5 | deletion | chr10: | 96489466-96554411 | 3.23E−11 |
| Father | deletion | chr11: | 18563533-18577561 | 4.79E−07 |
| Patient 4 | deletion | chr11: | 18562081-18579036 | 7.72E−08 |
| Patient 5 | deletion | chr11: | 18563533-18577561 | 4.84E−13 |
| Father | deletion | chr12: | 8493239-8667968 | 1.26E−34 |
| Patient 4 | deletion | chr12: | 8493239-8673506 | 1.64E−38 |
| Mother | duplication | chr12: | 50517243-50578630 | 2.55E−15 |
| Patient 4 | duplication | chr12: | 50517243-50577958 | 2.44E−15 |
| Patient 5 | duplication | chr12: | 50517243-50577958 | 0.00E+00 |
| Father | duplication | chr12: | 50975625-51067874 | 1.68E−14 |
| Father | duplication | chr12: | 69784529-69797993 | 7.13E−08 |
| Patient 4 | duplication | chr12: | 69785475-69805349 | 5.45E−08 |
| Patient 4 | duplication | chr15: | 50782920-50827258 | 0.00E+00 |
| Father | duplication | chr17: | 9929175-10356441 | 0.00E+00 |
| Patient 4 | duplication | chr17: | 9929175-10356441 | 0.00E+00 |

TABLE 10
qPCR primers

| Gene | Forward | Reverse |
|---|---|---|
| GAPDH | TGTTGCCATCAATGACCCCTT | CTCCACGACGTACTCAGCG |
| Actin | AAACTGGAACGGTGAAGGTG | AGAGAAGTGGGGTGGCTTTT |
| βIII-tubulin | ACCTCAACCACCTGGTATCG | TTCTTGGCATCGAACATCTG |
| GRIK1 | AAAGGTTACGGAGTGGGAAC | TCTTTGTTGTCTTCCTCGGG |
| GRIN2A | CTTGCTTCAGTTTGTGGGTG | AGCCAGCATGTAGAATACGC |
| GRM1 | AGCTTGTGACTTGGGATGG | TCGATGTTGCTCCACTCAAG |
| GRM7 | CCCGAGAATTTTAACGAAGCC | ATGGAGATTGTAAGCGTGGTAG |
| ADCY7 | CACTCCTTCAACTCCTTCCG | TCTCCAGTGCTTTCCATTCG |
| ADCY8 | TGCTGACTTCGATGAGTTGC | ATGTCCCCACTTGTCTTCAC |
| PDE3A | GCGATGAGTCAGGAGATACTG | AGAGGTGCTGAGTTATTTGGC |
| PDE4D | AGATAAGCCCCATGTGTGAC | CCTCCAAAGTGTCCAAAATATCC |
| PDE4DIP | CAGAAGGAGAGCATGGAACAG | ATGGTTCCTGGAAGGCAAG |
| PDE7B | TGCAATCCTTGTAGAATCTGGG | ACTAGGGATGGAATCTTTCTGTTG |
| PDE8A | GCATCCCCAAATCCCAAATC | TCATTTCGTCCAGTCCTTTCC |
| PDE10A | GAATTCTGGGCTGAGGGTG | GGGTAAGGGTTGTATAGCAGG |
| PRKCA | CACCATTCAAGCCCAAAGTG | CATACGAGAACCCTTCAAAATCAG |
| RAP2A | GGCTTCATCCTCGTCTACAG | TCACTTTCCAGGTCCACTTTG |
| RAP1A | AGTGTATGCTCGAAATCCTGG | AACGTGGACTGAGCTGTAATAG |
| WNT7A | AAGGTCTTTGTGGATGCCC | GCACTTACATTCCAGCTTCATG |
| LRP5 | CACTGCGAGACCGTACAG | GTCCGAGTTCAAATCCAGGTAG |
| AXIN2 | CAGAGGGACAGGAATCATTCG | AACCAACTCACTGGCCTG |
| TCF4 | GAACCTGCAAGACACGAAATC | CTTCTCACGCTCTGCCTTC |
| LEF1 | CATATGCAGCTTTATCCAGGC | CACCATGTTTCAGATGTAGGC |
| DISC1 | ACTCACCTCATCCCCTCTC | CACACTTTTCTCCAAGTTCTG |
| NRG1 | ACAAGGCACACAGATCCAAA | AAGGCCAAGGGGTCTTAGAG |
| NRG2 | CAGAAGAGGGTCCTGACCAT | GAGGTGGTTGTGCATCTGCT |
| NRG3 | AAAGGACCTGGTGGGCTATT | AGAATTCGGATCTGCTCCTG |
| ERBB4 | GGAGTATGTCCACGAGCACAA | TCGAGTCGTCTTTCTTCCAG |
| PSD-95 | ACAAGCGGATCACAGAGGAG | CAGATGTAGGGGCCTGAGAG |
| PSD-93 | AGCCTGTTACAAGGCAGGAA | GCCATCCACCTCGTAGTCTC |
| CYP2C19 | AAACGGATTTGTGTGGGAGA | ATAGAAGGGCGGGACAGAAG |
| GABRA6 | AAGGCTATGACAATCGGCTGC | TCAGTCCAGGTCTGGCGGAAA |
| GABRB2 | TGCCTGCATGATGGACCTAA | TCCTGTTACTGCATTATCAT |
| Lentiviral OCT4 | CCCCTGTCTCTGTCACCACT | CCACATAGCGTAAAAGGAGCA |
| Lentiviral SOX2 | ACACTGCCCCTCTCACACAT | CATAGCGTAAAAGGAGCAACA |
| Lentiviral cMYC | AAGAGGACTTGTTGCGGAAA | TTGTAATCCAGAGGTTGATTATCG |
| Lentiviral KLF4 | GACCACCTCGCCTTACACAT | CATAGCGTAAAAGGAGCAACA |
| Endogenous OCT4 | TGTACTCCTCGGTCCCTTTC | TCCAGGTTTTCTTTCCCTAGC |
| Endogenous SOX2 | GCTAGTCTCCAAGCGACGAA | GCAAGAAGCCTCTCCTTGAA |
| Endogenous cMYC | CGGAACTCTTGTGCGTAAGG | CTCAGCCAAGGTTGTGAGGT |
| Endogenous KLF4 | TATGACCCACACTGCCAGAA | TGGGAACTTGACCATGATTG |
| NANOG | CAGTCTGGACACTGGCTGAA | CTCGCTGATTAGGCTCCAAC |
| TDGF1 (CRIPTO) | AAGATGGCCCGCTTCTCTTAC | AGATGGACGAGCAAATTCCTG |
| ZFP42 (REX1) | AACGGGCAAAGACAAGACAC | GCTGACAGGTTCTATTTCCGC |

What is claimed is:

1. A method of determining whether a test compound is capable of improving a schizophrenia marker function in a hiPSC-derived neural cell, said method comprising:
(i) contacting a test compound with a hiPSC-derived neural cell, wherein said hiPSC-derived neural cell is derived from a schizophrenic subject, and wherein said hiPSC-derived neural cell exhibits a schizophrenia marker function at a first level in the absence of said test compound;
(ii) after step (i), determining a second level of said schizophrenia marker function; and
(iii) comparing the second level to a control level, wherein a smaller difference between the second level and the control level than between the first level and the control level indicates said test compound is capable of improving said schizophrenia marker function.

2. The method of claim 1, wherein said smaller difference indicates said schizophrenic subject is responsive to said test compound.

3. The method of claim 2, further comprising administering an effective amount of said test compound to said schizophrenic subject in need of treatment for schizophrenia.

4. The method of claim 1, wherein said hiPSC-derived neural cell is made by a method comprising:
(i) reprogramming a fibroblast cell thereby forming a fibroblast-derived hiPSC; and
(ii) differentiating said fibroblast-derived hiPSC thereby forming said hiPSC-derived neural cell.

5. The method of claim 4, wherein said fibroblast cell is obtained from a schizophrenic subject.

6. The method of claim 1, wherein said schizophrenia marker function is:
a number of neurites extending from said hiPSC-derived neural cell,
a level of PSD95 expressed by said hiPSC-derived neural cell,
a level of synaptic density of said hiPSC-derived neural cell,
a level of neural connectivity of said hiPSC-derived neural cell,
a level of synaptic plasticity of said hiPSC-derived neural cell,
a level of NRG1 expressed by said hiPSC-derived neural cell,
a level of a glutamate receptor expressed by said hiPSC-derived neural cell,
a level of a neuregulin pathway component expressed by said hiPSC-derived neural cell,
a level of a synaptic protein expressed by said hiPSC-derived neural cell,
a level of a cAMP component expressed by said hiPSC-derived neural cell,
a level of a calcium signaling pathway component expressed by said hiPSC-derived neural cell,
a level of a Wnt signaling pathway component expressed by said hiPSC-derived neural cell,
a level of a Notch growth factor expressed by said hiPSC-derived neural cell,
a level of neural migration of said hiPSC-derived neural cell, or
a level of a cell adhesion component expressed by said hiPSC-derived neural cell.

7. A method of determining whether a subject is schizophrenic, said method comprising:
(i) determining a level of a schizophrenia marker function in a hiPSC-derived neural cell derived from a subject;
(ii) comparing said level to a control level, wherein a difference between said level and said control level indicates said subject is schizophrenic.

8. The method of claim 7, further comprising:
(iii) quantitating said difference thereby determining a test quantity, and
(iv) comparing said test quantity to a control quantity thereby determining a severity of said subject's schizophrenia.

9. The method of claim 7, further comprising, prior to step (i):
(a) obtaining a cell from said subject;
(b) reprogramming said cell thereby forming a hiPSC;
(c) allowing said hiPSC to differentiate thereby forming a hiPSC-derived neural cell derived from said subject.

10. The method of claim 9, wherein said cell is a fibroblast cell.

11. The method of claim 9, further comprising treating said subject in need of treatment for schizophrenia.

12. A method of identifying a schizophrenia marker function, said method comprising:
(i) obtaining a cell from a schizophrenic subject;
(ii) reprogramming said cell thereby forming a hiPSC;
(iii) allowing said hiPSC to differentiate thereby forming a hiPSC-derived neural cell derived from said schizophrenic subject; and
(iv) determining a level of a function of said hiPSC-derived neural cell and comparing said level to a control level, wherein a difference between said level and said control level indicates said function is a schizophrenia marker function.

13. The method of claim 12, wherein said cell is a fibroblast cell.

14. A method of determining whether a schizophrenic subject is responsive to treatment with a loxapine compound, said method comprising:
(i) contacting a loxapine compound with a hiPSC-derived neural cell, wherein said hiPSC-derived neural cell is derived from said schizophrenic subject, and wherein said hiPSC-derived neural cell exhibits a loxapine marker function at a first level in the absence of loxapine;
(ii) after step (i), determining a second level of said loxapine marker function; and
(iii) comparing the second level to a control level, wherein a smaller difference between the second level and the control level than between the first level and the control level indicates said schizophrenic subject is responsive to treatment with a loxapine compound.

15. The method of claim 14, further comprising administering an effective amount of a loxapine compound to said schizophrenic subject in need of treatment for schizophrenia.

16. The method of claim 14, wherein said hiPSC-derived neural cell is made by a method comprising:
(i) reprogramming a fibroblast cell thereby forming a fibroblast-derived hiPSC; and
(ii) differentiating said fibroblast-derived hiPSC thereby forming said hiPSC-derived neural cell.

17. The method of claim 14, wherein said loxapine marker function is:
a level of a cytoskeleton remodeling component expressed by said hiPSC-derived neural cell,
a level of TGF signaling pathway component expressed by said hiPSC-derived neural cell,
a level of NRG1 expressed by said hiPSC-derived neural cell,
a level of a glutamate receptor expressed by said hiPSC-derived neural cell,
a level of neural connectivity of said hiPSC-derived neural cell, or
a level of a cell adhesion component expressed by said hiPSC-derived neural cell.

18. The method of claim 14, wherein said loxapine marker function is:
a level of a cytoskeleton remodeling component expressed by said hiPSC-derived neural cell,
a level of a TGF signaling pathway component expressed by said hiPSC-derived neural cell,
a level of NRG1 expressed by said hiPSC-derived neural cell,
a level of a glutamate receptor expressed by said hiPSC-derived neural cell,
a level of neural connectivity of said hiPSC-derived neural cell, and
a level of a cell adhesion component expressed by said hiPSC-derived neural cell.

19. A method of determining whether a test compound is capable of improving a loxapine marker function, said method comprising:
(i) contacting a test compound with a hiPSC-derived neural cell, wherein said hiPSC-derived neural cell is derived from a schizophrenic subject, and wherein said hiPSC-derived neural cell exhibits a loxapine marker function at a first level in the absence of said test compound;
(ii) after step (i), determining a second level of said loxapine marker function; and
(iii) comparing the second level to a control level, wherein a smaller difference between the second level and the control level than between the first level and the control level indicates said test compound is capable of improving said loxapine marker function.

20. The method of claim 19, wherein said smaller difference indicates said schizophrenic subject is responsive to said test compound.

21. The method of claim 20, further comprising administering an effective amount of said test compound to said schizophrenic subject in need of treatment for schizophrenia.

22. The method of claim 19, wherein said hiPSC-derived neural cell is made by a method comprising:
(i) reprogramming a fibroblast cell thereby forming a fibroblast-derived hiPSC; and
(ii) differentiating said fibroblast-derived hiPSC thereby forming said hiPSC-derived neural cell.

23. The method of claim 22, wherein said fibroblast cell is obtained from a schizophrenic subject.

24. The method of claim 19, wherein said loxapine marker function is:
a level of a cytoskeleton remodeling component expressed by said hiPSC-derived neural cell,
a level of TGF signaling pathway component expressed by said hiPSC-derived neural cell,
a level of NRG1 expressed by said hiPSC-derived neural cell,
a level of a glutamate receptor expressed by said hiPSC-derived neural cell,
a level of neural connectivity of said hiPSC-derived neural cell, or
a level of a cell adhesion component expressed by said hiPSC-derived neural cell.


SEQUENCE LISTING (88 sequences)

SEQ ID NO: 1 - DNA, 21 bp, Artificial Sequence
  Synthetic DNA forward primer
tgttgccatcaatgacccctt

SEQ ID NO: 2 - DNA, 19 bp, Artificial Sequence
  Synthetic DNA reverse primer
ctccacgacgtactcagcg

SEQ ID NO: 3 - DNA, 20 bp, Artificial Sequence
  Synthetic DNA forward primer
aaactggaacggtgaaggtg

SEQ ID NO: 4 - DNA, 20 bp, Artificial Sequence
  Synthetic DNA reverse primer
agagaagtggggtggctttt

SEQ ID NO: 5 - DNA, 20 bp, Artificial Sequence
  Synthetic DNA forward primer
acctcaaccacctggtatcg

SEQ ID NO: 6 - DNA, 20 bp, Artificial Sequence
  Synthetic DNA reverse primer
ttcttggcatcgaacatctg

SEQ ID NO: 7 - DNA, 20 bp, Artificial Sequence
  Synthetic DNA forward primer
aaaggttacggagtgggaac

SEQ ID NO: 8 - DNA, 20 bp, Artificial Sequence
  Synthetic DNA reverse primer
tctttgttgtcttcctcggg

SEQ ID NO: 9 - DNA, 20 bp, Artificial Sequence
  Synthetic DNA forward primer
cttgcttcagtttgtgggtg

SEQ ID NO: 10 - DNA, 20 bp, Artificial Sequence
  Synthetic DNA reverse primer
agccagcatgtagaatacgc

SEQ ID NO: 11 - DNA, 19 bp, Artificial Sequence
  Synthetic DNA forward primer
agcttgtgacttgggatgg

SEQ ID NO: 12 - DNA, 20 bp, Artificial Sequence
  Synthetic DNA reverse primer
tcgatgttgctccactcaag

SEQ ID NO: 13 - DNA, 21 bp, Artificial Sequence
  Synthetic DNA forward primer
cccgagaattttaacgaagcc

SEQ ID NO: 14 - DNA, 22 bp, Artificial Sequence
  Synthetic DNA reverse primer
atggagattgtaagcgtggtag

SEQ ID NO: 15 - DNA, 20 bp, Artificial Sequence
  Synthetic DNA forward primer
cactccttcaactccttccg

SEQ ID NO: 16 - DNA, 20 bp, Artificial Sequence
  Synthetic DNA reverse primer
tctccagtgctttccattcg

SEQ ID NO: 17 - DNA, 20 bp, Artificial Sequence
  Synthetic DNA forward primer
tgctgacttcgatgagttgc

SEQ ID NO: 18 - DNA, 20 bp, Artificial Sequence
  Synthetic DNA reverse primer
atgtccccacttgtcttcac

SEQ ID NO: 19 - DNA, 21 bp, Artificial Sequence
  Synthetic DNA forward primer
gcgatgagtcaggagatactg

SEQ ID NO: 20 - DNA, 21 bp, Artificial Sequence
  Synthetic DNA reverse primer
agaggtgctgagttatttggc

SEQ ID NO: 21 - DNA, 20 bp, Artificial Sequence
  Synthetic DNA forward primer
agataagccccatgtgtgac

SEQ ID NO: 22 - DNA, 23 bp, Artificial Sequence
  Synthetic DNA reverse primer
cctccaaagtgtccaaaatatcc

SEQ ID NO: 23 - DNA, 21 bp, Artificial Sequence
  Synthetic DNA forward primer
cagaaggagagcatggaacag

SEQ ID NO: 24 - DNA, 19 bp, Artificial Sequence
  Synthetic DNA reverse primer
atggttcctggaaggcaag

SEQ ID NO: 25 - DNA, 22 bp, Artificial Sequence
  Synthetic DNA forward primer
tgcaatccttgtagaatctggg

SEQ ID NO: 26 - DNA, 24 bp, Artificial Sequence
  Synthetic DNA reverse primer
actagggatggaatctttctgttg

SEQ ID NO: 27 - DNA, 20 bp, Artificial Sequence
  Synthetic DNA forward primer
gcatccccaaatcccaaatc

SEQ ID NO: 28 - DNA, 21 bp, Artificial Sequence
  Synthetic DNA reverse primer
tcatttcgtccagtcctttcc

SEQ ID NO: 29 - DNA, 19 bp, Artificial Sequence
  Synthetic DNA forward primer
gaattctgggctgagggtg

SEQ ID NO: 30 - DNA, 21 bp, Artificial Sequence
  Synthetic DNA reverse primer
gggtaagggttgtatagcagg

SEQ ID NO: 31 - DNA, 20 bp, Artificial Sequence
  Synthetic DNA forward primer
caccattcaagcccaaagtg

SEQ ID NO: 32 - DNA, 24 bp, Artificial Sequence
  Synthetic DNA reverse primer
catacgagaacccttcaaaatcag

SEQ ID NO: 33 - DNA, 20 bp, Artificial Sequence
  Synthetic DNA forward primer
ggcttcatcctcgtctacag

SEQ ID NO: 34 - DNA, 21 bp, Artificial Sequence
  Synthetic DNA reverse primer
tcactttccaggtccactttg

SEQ ID NO: 35 - DNA, 21 bp, Artificial Sequence
  Synthetic DNA forward primer
agtgtatgctcgaaatcctgg

SEQ ID NO: 36 - DNA, 22 bp, Artificial Sequence
  Synthetic DNA reverse primer
aacgtggactgagctgtaatag

SEQ ID NO: 37 - DNA, 19 bp, Artificial Sequence
  Synthetic DNA forward primer
aaggtctttgtggatgccc

SEQ ID NO: 38 - DNA, 22 bp, Artificial Sequence
  Synthetic DNA reverse primer
gcacttacattccagcttcatg

SEQ ID NO: 39 - DNA, 18 bp, Artificial Sequence
  Synthetic DNA forward primer
cactgcgagaccgtacag

SEQ ID NO: 40 - DNA, 22 bp, Artificial Sequence
  Synthetic DNA reverse primer
gtccgagttcaaatccaggtag

SEQ ID NO: 41 - DNA, 21 bp, Artificial Sequence
  Synthetic DNA forward primer
cagagggacaggaatcattcg

SEQ ID NO: 42 - DNA, 18 bp, Artificial Sequence
  Synthetic DNA reverse primer
aaccaactcactggcctg

SEQ ID NO: 43 - DNA, 21 bp, Artificial Sequence
  Synthetic DNA forward primer
gaacctgcaagacacgaaatc

SEQ ID NO: 44 - DNA, 19 bp, Artificial Sequence
  Synthetic DNA reverse primer
cttctcacgctctgccttc

SEQ ID NO: 45 - DNA, 21 bp, Artificial Sequence
  Synthetic DNA forward primer
catatgcagctttatccaggc

SEQ ID NO: 46 - DNA, 21 bp, Artificial Sequence
  Synthetic DNA reverse primer
caccatgtttcagatgtaggc

SEQ ID NO: 47 - DNA, 19 bp, Artificial Sequence
  Synthetic DNA forward primer
actcacctcatcccctctc

SEQ ID NO: 48 - DNA, 21 bp, Artificial Sequence
  Synthetic DNA reverse primer
cacacttttctccaagttctg

SEQ ID NO: 49 - DNA, 20 bp, Artificial Sequence
  Synthetic DNA forward primer
acaaggcacacagatccaaa

SEQ ID NO: 50 - DNA, 20 bp, Artificial Sequence
  Synthetic DNA reverse primer
aaggccaaggggtcttagag

SEQ ID NO: 51 - DNA, 20 bp, Artificial Sequence
  Synthetic DNA forward primer
cagaagagggtcctgaccat

SEQ ID NO: 52 - DNA, 20 bp, Artificial Sequence
  Synthetic DNA reverse primer
gaggtggttgtgcatctgct

SEQ ID NO: 53 - DNA, 20 bp, Artificial Sequence
  Synthetic DNA forward primer
aaaggacctggtgggctatt

SEQ ID NO: 54 - DNA, 20 bp, Artificial Sequence
  Synthetic DNA reverse primer
agaattcggatctgctcctg

SEQ ID NO: 55 - DNA, 21 bp, Artificial Sequence
  Synthetic DNA forward primer
ggagtatgtccacgagcacaa

SEQ ID NO: 56 - DNA, 20 bp, Artificial Sequence
  Synthetic DNA reverse primer
tcgagtcgtctttcttccag

SEQ ID NO: 57 - DNA, 20 bp, Artificial Sequence
  Synthetic DNA forward primer
acaagcggatcacagaggag

SEQ ID NO: 58 - DNA, 20 bp, Artificial Sequence
  Synthetic DNA reverse primer
cagatgtaggggcctgagag

SEQ ID NO: 59 - DNA, 20 bp, Artificial Sequence
  Synthetic DNA forward primer
agcctgttacaaggcaggaa

SEQ ID NO: 60 - DNA, 20 bp, Artificial Sequence
  Synthetic DNA reverse primer
gccatccacctcgtagtctc

SEQ ID NO: 61 - DNA, 20 bp, Artificial Sequence
  Synthetic DNA forward primer
aaacggatttgtgtgggaga

SEQ ID NO: 62 - DNA, 20 bp, Artificial Sequence
  Synthetic DNA reverse primer
atagaagggcgggacagaag

SEQ ID NO: 63 - DNA, 21 bp, Artificial Sequence
  Synthetic DNA forward primer
aaggctatgacaatcggctgc

SEQ ID NO: 64 - DNA, 21 bp, Artificial Sequence
  Synthetic DNA reverse primer
tcagtccaggtctggcggaaa

SEQ ID NO: 65 - DNA, 20 bp, Artificial Sequence
  Synthetic DNA forward primer
tgcctgcatgatggacctaa

SEQ ID NO: 66 - DNA, 20 bp, Artificial Sequence
  Synthetic DNA reverse primer
tcctgttactgcattatcat

SEQ ID NO: 67 - DNA, 20 bp, Artificial Sequence
  Synthetic DNA forward primer
cccctgtctctgtcaccact

SEQ ID NO: 68 - DNA, 21 bp, Artificial Sequence
  Synthetic DNA reverse primer
ccacatagcgtaaaaggagca

SEQ ID NO: 69 - DNA, 20 bp, Artificial Sequence
  Synthetic DNA forward primer
acactgcccctctcacacat

SEQ ID NO: 70 - DNA, 21 bp, Artificial Sequence
  Synthetic DNA reverse primer
catagcgtaaaaggagcaaca

SEQ ID NO: 71 - DNA, 20 bp, Artificial Sequence
  Synthetic DNA forward primer
aagaggacttgttgcggaaa

SEQ ID NO: 72 - DNA, 24 bp, Artificial Sequence
  Synthetic DNA reverse primer
ttgtaatccagaggttgattatcg

SEQ ID NO: 73 - DNA, 20 bp, Artificial Sequence
  Synthetic DNA forward primer
gaccacctcgccttacacat

SEQ ID NO: 74 - DNA, 21 bp, Artificial Sequence
  Synthetic DNA reverse primer
catagcgtaaaaggagcaaca

SEQ ID NO: 75 - DNA, 20 bp, Artificial Sequence
  Synthetic DNA forward primer
tgtactcctcggtccctttc

SEQ ID NO: 76 - DNA, 21 bp, Artificial Sequence
  Synthetic DNA reverse primer
tccaggttttctttccctagc

SEQ ID NO: 77 - DNA, 20 bp, Artificial Sequence
  Synthetic DNA forward primer
gctagtctccaagcgacgaa

SEQ ID NO: 78 - DNA, 20 bp, Artificial Sequence
  Synthetic DNA reverse primer
gcaagaagcctctccttgaa

SEQ ID NO: 79 - DNA, 20 bp, Artificial Sequence
  Synthetic DNA forward primer
cggaactcttgtgcgtaagg

SEQ ID NO: 80 - DNA, 20 bp, Artificial Sequence
  Synthetic DNA reverse primer
ctcagccaaggttgtgaggt

SEQ ID NO: 81 - DNA, 20 bp, Artificial Sequence
  Synthetic DNA forward primer
tatgacccacactgccagaa

SEQ ID NO: 82 - DNA, 20 bp, Artificial Sequence
  Synthetic DNA reverse primer
tgggaacttgaccatgattg

SEQ ID NO: 83 - DNA, 20 bp, Artificial Sequence
  Synthetic DNA forward primer
cagtctggacactggctgaa

SEQ ID NO: 84 - DNA, 20 bp, Artificial Sequence
  Synthetic DNA reverse primer
ctcgctgattaggctccaac

SEQ ID NO: 85 - DNA, 21 bp, Artificial Sequence
  Synthetic DNA forward primer
aagatggcccgcttctcttac

SEQ ID NO: 86 - DNA, 21 bp, Artificial Sequence
  Synthetic DNA reverse primer
agatggacgagcaaattcctg

SEQ ID NO: 87 - DNA, 20 bp, Artificial Sequence
  Synthetic DNA forward primer
aacgggcaaagacaagacac

SEQ ID NO: 88 - DNA, 21 bp, Artificial Sequence
  Synthetic DNA reverse primer
gctgacaggttctatttccgc